SEQUENCE LISTING (102 sequences)

SEQ ID NO: 1 - DNA, 1490 bp, Pseudomonas fluorescens
  source 1..1490: mol_type = genomic DNA
agtcagacatgcaagtcgagcggtagagagaagcttgcttctcttgagagcggcggacgggtgagtaaagcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 2 - DNA, 1017 bp, Hanseniaspora occidentalis
  source 1..1017: mol_type = genomic DNA
ttgttgctcgagttcttgtttagatcttttacaataatgtgtatctttaatgaagatgngngcttaattgcgctgctttattagagtgtcgcagtagaagtagtcttgcttgaatctcagtcaacgtttacacacattggagtttttttactttaatttaattctttctgctttgaatcgaaaggttcaaggcaaaaaacaaacacaaacaattttattttattataattttttaaactaaaccaaaattcctaacggaaattttaaaataatttaaaactttcaacaacggatctcttggttctcgcatcgatgaaaaacgtaccgaattgcgataagtaatgtgaattgcaaatactcgtgaatcattgaatttttgaacgcacattgcgcccttgagcattctcaagggcatgcctgtttgagcgtcatttccttctcaaaaaataattttttattttttggttgtgggcgatactcagggttagcttgaaattggagactgtttcagtcttttttaattcaacacttancttctttggagacgctgttctcgctgtgatgtatttatggatttattcgttttactttacaagggaaatggtaatgtaccttaggcaaagggttgcttttaatattcatcaagtttgacctcaaatcaggtaggattacccgctgaacttaagcatatcaataagcggaggaaaagaaaccaactgggattaccttagtaacggcgagtgaagcggtaaaagctcaaatttgaaatctggtactttcagtgcccgagttgtaatttgtagaatttgtctttgattaggtccttgtctatgttccttggaacaggacgtcatagagggtgagantcccgtttgnngaggataccttttctctgtannactttttcnaagagtcgagttgnttgggaatgcagctcaaanngggtngnaaattccatctaaagctaaatattngncnagagaccganagcgacantacagngatggaaagangaaa

SEQ ID NO: 3 - DNA, 1548 bp, Leuconostoc mesenteroides
  source 1..1548: mol_type = genomic DNA
attgagagtttgatcctggctcaggatgaacgctggcggcgtgcctaatacatgcaagtcgaacgcacagcgaaaggtgcttgcacctttcaagtgagtggcgaacgggtgagtaacacgtggacaacctgcctcaaggctggggataacatttggaaacagatgctaataccgaataaaactcagtgtcgcatgacacaaagttaaaaggcgctttggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtacgggaagaacagctagaatagggaatgattttagtttgacggtaccataccagaaagggacggctaaatacgtgccagcagccgcggtaatacgtatgtcccgagcgttatccggatttattgggcgtaaagcgagcgcagacggttgattaagtctgatgtgaaagcccggagctcaactccggaatggcattggaaactggttaacttgagtgcagtagaggtaagtggaactccatgtgtagcggtggaatgcgtagatatatggaagaacaccagtggcgaaggcggcttactggactgtaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggattgtagtctgcaactcgactacatgaagtcggaatcgctagtaatcgcggatcagcacgccgcggtgaatacgttcccgggtcttgtacacaccgcccgtcacaccatgggagtttgtaatgcccaaagccggtggcctaaccttttaggaaggagccgtctaaggcaggacagatgactggggtgaagtcgtaacaaggtagccgtaggagaacctgcggctggatcacctccttt

SEQ ID NO: 4 - DNA, 916 bp, Aureobasidium pullulans
  source 1..916: mol_type = genomic DNA
ctttgttgttaaaactaccttgttgctttggcgggaccgctcggtctcgagccgctggggattcgtcccaggcgagcgcccgccagagttaaaccaaactcttgttatttaaccggtcgtctgagttaaaattttgaataaatcaaaactttcaacaacggatctcttggttctcgcatcgatgaagaacgcagcgaaatgcgataagtaatgtgaattgcagaattcagtgaatcatcgaatctttgaacgcacattgcgccccttggtattccgaggggcatgcctgttcgagcgtcattacaccactcaagctatgcttggtattgggcgtcgtccttagttgggcgcgccttaaagacctcggcgaggccactccggctttaggcgtagtagaatttattcgaacgtctgtcaaaggagaggaactctgccgactgaaacctttatttttctaggttgacctcggatcaggtagggatacccgctgaacttaagcatatcaataagcggaggaaaagaaaccaacagggattgccctagtaacggcgagtgaagcggcaacagctcaaatttgaaagctagccttcgggttcgcattgtaatttgtagaggatgatttggggaagccgcctgtctaagttccttggaacaggacgtcatagagggtgagaatcccgtatgtgacaggaaatggcaccctatgtaaatctccttcgacgagtcgagttgtttgggaatgcagctctaaatgggaggtaaatttcttctaaagctaaatattggcgagagaccgatagcgcacaagtagagtgatcgaaagatgaaaagcactttggaaagagagttaaaaagcacgtgaaattgttgaaagggaagcgcttgcaatcagacttgtttaaactgttcggccggt

SEQ ID NO: 5 - DNA, 1042 bp, Debaromyces hansenii
  source 1..1042: mol_type = genomic DNA
gcgcttattgcgcggcgaaaaaaccttacacacagtgttttttgttattacannaacttttgctttggtctggactagaaatagtttgggccagaggttactaaactaaacttcaatatttatattgaattgttatttatttaattgtcaatttgttgattaaattcaaaaaatcttcaaaactttcaacaacggatctcttggttctcgcatcgatgaagaacgcagcgaaatgcgataagtaatatgaattgcagattttcgtgaatcatcgaatctttgaacgcacattgcgccctctggtattccagagggcatgcctgtttgagcgtcatttctctctcaaaccttcgggtttggtattgagtgatactcttagtcgaactaggcgtttgcttgaaatgtattggcatgagtggtactggatagtgctatatgactttcaatgtattaggtttatccaactcgttgaatagtttaatggtatatttctcggtattctaggctcggccttacaatataacaaacaagtttgacctcaaatcaggtaggattacccgctgaacttaagcatatcaataagcggaggaaaagaaaccaacagggattgccttagtaacggcgagtgaagcggcaaaagctcaaatttgaaatctggcaccttcggtgtccgagttgtaatttgaagaaggtaactttggagttggctcttgtctatgttccttggaacaggacgtcacagagggtgagaatcccgtgcgatgagatgcccaattctatgtaaagtgctttcgaagagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaaagggcttgagatcagacttggtattttgcgatcctttccttcttggttgggttcctcgcagcttactgggncagcatcggtttggatgg

SEQ ID NO: 6 - DNA, 1165 bp, Bacillus wiedmannii
  source 1..1165: mol_type = genomic DNA
gaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctgcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcat

SEQ ID NO: 7 - DNA, 871 bp, Pichia fermentans
  source 1..871: mol_type = genomic DNA
ccacnctgcgtgggcgacacgaaacaccgaaaccgaacgcacgccgtcaagcaagaaatccacaaaactttcaacaacggatctcttggttctcgcatcgatgaagagcgcagcgaaatgcgatacctagtgtgaattgcagccatcgtgaatcatcgagttcttgaacgcacattgcgcccgctggtattccggcgggcatgcctgtctgagcgtcgtttccttcttggagcggagcttcagacctggcgggctgtctttcgggacggcgcgcccaaagcgaggggccttctgcgcgaactagactgtgcgcgcggggcggccggcgaacttataccaagctcgacctcagatcaggcaggagtacccgctgaacttaagcatatcaataagcggaggaaaagaaaccaacagggattgccccagtagcggcgagtgaagcggcaaaagctcagatttggaatcgcttcggcgagttgtgaattgcaggttggcgcctctgcggcggcggcggtccaagtcccttggaacagggcgccattgagggtgagagccccgtgggaccgtttgcctatgctctgaggcccttctgacgagtcgagttgtttgggaatgcagctctaagcgggtggtaaattccatctaaggctaaatactggcgagagaccgatagcgaacaagtactgtgaaggaaagatgaaaagcactttgaaaagagagtgaaacagcacgtgaaattgttgaaagggaagggtattgcgcccgacatggagcgtgcgcaccgctgcccctcgtgggcggcgctctgggcgtgctctgggccagcatcggtttttgccgcgggagaagggcggcgggcatgtagctcttc

SEQ ID NO: 8 - DNA, 942 bp, Hanseniaspora opuntiae
  source 1..942: mol_type = genomic DNA
gttgctcgagttcttgtttagatcttttacnataatgtgtatctttaatgaagatgtgcgcttaattgcgctgctttattagagtgtcgcagtagaagtagtcttgcttgaatctcagtcaacgtttacacacattggagtttttttactttaatttaattctttctgctttgaatcgaaaggttcaaggcaaaaaacaaacacaaacaattttattttattataattttttaaactaaaccaaaattcctaacggaaattttaaaataatttaaaactttcaacaacggatctcttggttctcgcatcgatgaaaaacgtagcgaattgcgataagtaatgtgaattgcaaatactcgtgaatcattgaatttttgaacgcacattgcgcccttgagcattctcaagggcatgcctgtttgagcgtcatttccttctcaaaagataattttttattttttggttgtgggcgatactcagggttagcttgaaattggagactgtttcagtcttttttaattcaacacttancttctttggagacgctgttctcgctgtgatgtatttatggatttattcgttttactttacaagggaaatggtaatgtaccttaggcaaagggttgcttttaatattcatcaagtttgacctcaaatcaggtaggattacccgctgaacttaagcatatcaataagcggaggaaaagaaaccaactgggattaccttagtaacggcgagtgaagcggtaaaagctcaaatttgaaatctggtactttcanngcccgagttgtaatttgtagaatttgtctttgattaggtccttgtctatgttccttggancaggacgtcatanagggtgantcccntttggcganganaccttttctctgtanactttttcnanagtcgagttgtttnggatgcagctcnaagtggggngg

SEQ ID NO: 9 - DNA, 1572 bp, Pediococcus pentosaceus
  source 1..1572: mol_type = genomic DNA
atgagagtttgatcttggctcaggatgaacgctggcggcgtgcctaatacatgcaagtcgaacgaacttccgttaattgattatgacgtacttgtactgattgagattttaacacgaagtgagtggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggcaaaggctcaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgagaccgcgaggttaagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagtcgtaacaaggtagccgtaggagaacctgcggctggatcacctcctt

SEQ ID NO: 10 - DNA, 1300 bp, Bacillus velezensis
  source 1..1300: mol_type = genomic DNA
cagatagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 11 - DNA, 1419 bp, Pseudomonas putida
  source 1..1419: mol_type = genomic DNA
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggttcgttaagttggatgtgaaagccccgggctcaacctgggaactgcattcaaaactgacgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggatgggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacatcccacacgaattgcttg

SEQ ID NO: 12 - DNA, 1520 bp, Microbacterium sp.
  source 1..1520: mol_type = genomic DNA
tacggagagtttgatcctggctcaggatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagccttcatcgcatggtgggggttggaaagattttttggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggaccattccacggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatacaccagaacgggccagaaatggtcaactctttggacactggtgaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcgtaacaaggtagccgtaccggaaggtgcggctggatcacctccttt

SEQ ID NO: 13 - DNA, 1457 bp, Bacillus mycoides
  source 1..1457: mol_type = genomic DNA
agttagcggcggacgggtgagtaacacgtgggtaacctgcctataagactgggataactccgggaaaccggggctaataccggataacattttgcaccgcatggtgcgaaattgaaaggcggcttcggctgtcacttatagatggacctgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgatgaaggctttcgggtcgtaaagttctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgcaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttccccttcgggggcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagtcgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaaccttttggagccagccgcctaaggtgggacagatgattggggtgaagtcgtaacaaggtagccgtatcggaaggtgcggctggatcacctccttt

SEQ ID NO: 14 - DNA, 1526 bp, Arthrobacter luteolus
  source 1..1526: mol_type = genomic DNA
tacggagagtttgatcctggctcaggatgaacgctggcggcgtgcttaacacatgcaagtcgaacgatgacttctgtgcttgcacagaatgattagtggcgaacgggtgagtaacacgtgagtaacctgcccttaacttcgggataagcctgggaaaccgggtctaataccggatacgacctcctggcgcatgccatggtggtggaaagctttagcggttttggatggactcgcggcctatcagcttgttggttggggtaatggcccaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttatccggaattattgggcgtaaagagctcgtaggcggtttgtcgcgtctgctgtgaaagcccggggctcaaccccgggtctgcagtgggtacgggcagactagagtgcagtaggggagactggaattcctggtgtagcggtgaaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaagacctggaaacaggtcccccacttgtggccggtttacaggtggtgcatggttgtcgtcagctcgtgtcgtgagatgttgggttaagtcccgcaacgagcgcaaccctcgttctatgttgccagcgggttatgccggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattgaggtctgcaactcgacctcatgaagttggagtcgctagtaatcgcagatcagcaacgctgcggtgaatacgttcccgggccttgtacacaccgcccgtcaagtcacgaaagttggtaacacccgaagccggtggcctaaccccttgtgggagggagccgtcgaaggtgggaccggcgattgggactaagtcgtaacaaggtagccgtaccggaaggtgcggctggatcacctccttt

SEQ ID NO: 15 - DNA, 1520 bp, Curtobacterium sp.
  source 1..1520: mol_type = genomic DNA
tacggagagtttgatcctggctcaggacgaacgctggcggcgtgcttaacacatgcaagtcgaacgatgatcaggagcttgctcctgtgattagtggcgaacgggtgagtaacacgtgagtaacctgcccctgactctgggataagcgttggaaacgacgtctaatactggatatgatcactggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcgtggggagcgaacaggattagataccctggtagtccacgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtcgaaggtgggatcggtgattaggactaagtcgtaacaaggtagccgtaccggaaggtgcggctggatcacctccttt

SEQ ID NO: 16 - skipped

SEQ ID NO: 17 - DNA, 898 bp, Rahnella aquatilis
  source 1..898: mol_type = genomic DNA
gtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaattcgccagagatggcttagtgccttcgggaaccgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacgg

SEQ ID NO: 18 - DNA, 1532 bp, Pseudomonas sp.
  source 1..1532: mol_type = genomic DNA
tgaagagtttgatcatggctcagattgaacgctggcggcaggcctaacacatgcaagtcgagcggatgaaaggagcttgctcctggattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaaattaatactttgctgttttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 19 - DNA, 1526 bp, Curtobacterium pusillum
  source 1..1526: mol_type = genomic DNA
tacggagagtttgatcctggctcaggacgaacgctggcggcgtgcttaacacatgcaagtcgaacgatgatgcccagcttgctgggtggattagtggcgaacgggtgagtaacacgtgagtaacctgcccctgactctgggataagcgttggaaacgacgtctaatactggatatgactgccggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagggagcttgctcttgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcatggggagcgaacaggattagataccctggtagtccatgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtcgaaggtgggatcggtgattaggactaagtcgtaacaaggtagccgtaccggaaggtgcggctggatcacctccttt

SEQ ID NO: 20 - DNA, 1543 bp, Stenotrophomonas rhizophila
  source 1..1543: mol_type = genomic DNA
tgaagagtttgatcctggctcagagtgaacgctggcggtaggcctaacacatgcaagtcgaacggcagcacagtaagagcttgctcttatgggtggcgagtggcggacgggtgaggaatacatcggaatctaccttttcgtgggggataacgtagggaaacttacgctaataccgcatacgaccttcgggtgaaagcaggggaccttcgggccttgcgcggatagatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccataccgcgtgggtgaagaaggccttcgggttgtaaagcccttttgttgggaaagaaaagcagtcggctaatacccggttgttctgacggtacccaaagaataagcaccggctaacttcgtgccagcagccgcggtaatacgaagggtgcaagcgttactcggaattactgggcgtaaagcgtgcgtaggtggttgtttaagtctgttgtgaaagccctgggctcaacctgggaattgcagtggatactgggcgactagagtgtggtagagggtagtggaattcccggtgtagcagtgaaatgcgtagagatcgggaggaacatccatggcgaaggcagctacctggaccaacactgacactgaggcacgaaagcgtggggagcaaacaggattagataccctggtagtccacgccctaaacgatgcgaactggatgttgggtgcaatttggcacgcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggtcttgacatgtcgagaactttccagagatggattggtgccttcgggaactcgaacacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacgaccagggctacacacgtactacaatggtagggacagagggctgcaaacccgcgagggcaagccaatcccagaaaccctatctcagtccggattggagtctgcaactcgactccatgaagtcggaatcgctagtaatcgcagatcagcattgctgcggtgaatacgttcccgggccttgtacacaccgcccgtcacaccatgggagtttgttgcaccagaagcaggtagcttaaccttcgggagggcgcttgccacggtgtggccgatgactggggtgaagtcgtaacaaggtagccgtatcggaaggtgcggctggatcacctccttt

SEQ ID NO: 21 - DNA, 927 bp, Candida santamariae
  source 1..927: mol_type = genomic DNA
gggggcatcagtattcagttgtcagaggtgaaattcttggatttactgaagactaactactgcgaaagcatttgccaaggacgttttcattaatcaagaacgaaagttaggggatcgaagatgatcagataccgtcgtagtcttaaccataaactatgccgactagggatcgggtgttgttctttttttgacgcactcggcaccttacgagaaatcaaagtctttgggttctggggggagtatggtcgcaaggctgaaacttaaaggaattgacggaagggcaccaccaggagtggagcctgcggcttaatttgactcaacacggggaaactcaccaggtccagacacaataaggattgacagattgagagctctttcttgattttgtgggtggtggtgcatggccgttcttagttggtggagtgatttgtctgcttaattgcgataacgaacgagaccttaacctactaaatagtgctgctagcttttgctggtatagtcacttcttagagggactatcgatttcaagtcgatggaagtttgaggcaataacaggtctgtgatgcccttagacgttctgggccgcacgcgcgctacactgacggagccagcgagttctaaccttggccgagaggtctgggtaatcttgtgaaactccgtcgtgctggggatagagcattgtaattattgctcttcaacgaggaattcctagtaagcgcaagtcatcagcttgcgttgattacgtccctgccctttgtacacaccgcccgtcgctactaccgattgaatggcttagtgaggcttccggattggtttaaagaagggggcaactccatcttggaaccgaaaagctagtcaaacttggtcatttagaggaagtaaaagtcgtaacaaggtttccgtaggtgaacctgcggaaggatcatt

SEQ ID NO: 22 - DNA, 1539 bp, Rahnella sp.
  source 1..1539: mol_type = genomic DNA
ttgaagagtttgatcatggctcagattgaacgctggcggcaggcctaacacatgcaagtcgagcggtagcacaggagagcttgctctccgggtgacgagcggcggacgggtgagtaatgtctgggaaactgcctgatggagggggataactactggaaacggtagctaataccgcatgacgtcgcaagaccaaagtgggggaccttcgggcctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcgttgcagttaatagctgcagcgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 23 - DNA, 1536 bp, Erwinia billingiae
  source 1..1536: mol_type = genomic DNA
ttgaagagtttgatcatggctcagattgaacgctggcggcaggcctaacacatgcaagtcgaacggtagcacagagagcttgctcttgggtgacgagtggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatacggttaataaccgtgtcgattgacgttacccgcagaagaagcaccggctaactccgtgccagcagccgcggtaatacggagggtgcaagcgttaatcggaattactgggcgtaaagcgcacgcaggcggtctgtcaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccacagaattcggcagagatgccttagtgccttcgggaactgtgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 24 - DNA, 1441 bp, Filobasidium globisporum
  source 1..1441: mol_type = genomic DNA
cggggaattagggttcgattccggagagggagcctgagaaacggctaccacatccaaggaaggcagcaggcgcgcaaattacccaatcccgacacggggaggtagtgacaataaataacaatacagggccctttgggtcttgtaattggaatgagtacaatttaaatcccttaacgaggaacaattggagggcaagtctggtgccagcagccgcggtaattccagctccaatagcgtatattaaagttgttgcagttaaaaagctcgtagttgaacttcaggcttggcggggtggtctgcctcacggtatgtactatccggctgagccttacctcctggtgagcctgcatgtcgtttattcggtgtgtaggggaaccaggaattttactttgaaaaaattagagtgttcaaagcaggcatatgcccgaatacattagcatggaataatagaataggacgtgcggttctattttgttggtttctaggatcgccgtaatgattaatagggacggttgggggcattagtattcagttgctagaggtgaaattcttagatttactgaagactaactactgcgaaagcatttgccaaggacgttttcattaatcaagaacgaaggttaggggatcaaaaacgattagataccgttgtagtcttaacagtaaactatgccgactagggatcgggccacgttcatcttttgactggctcggcaccttacgagaaatcaaagtctttgggttctggggggagtatggtcgcaaggctgaaacttaaaggaattgacggaagggcaccaccaggcgtggagcctgcggcttaatttgactcaacacggggaaactcaccaggtccagacatagtaaggattgacagattgatagctctttcttgattctatgggtggtggtgcatggccgttcttagttggtggagtgatttgtctggttaattccgataacgaacgagaccttaacctgctaaatagtccggccggcttcggctggtcgctgacttcttagagggactaacagcgtttagctgttggaagtttgaggcaataacaggtctgtgatgcccttagatgttctgggccgcacgcgcgctacactgactgagccagcgagtttataaccttggccgaaaggtctgggtaatcttgtgaaactcagtcgtgctggggatagagcattgcaattattgctcttcaacgaggaatgcctagtaagcgtgagtcatcagctcacgttgattacgtccctgccctttgtacacaccgcccgtcgctactaccgattgaatggcttagtgagatctccggattggctttgggaagctggcaacggctacctattgctgaaaagctgatcaaacttggtcatttagaggaagtaaaagtcgtaacaaggtttccgtaggtgaacctgcggaaggatcatt

SEQ ID NO: 25 - skipped

SEQ ID NO: 26 - DNA, 1482 bp, Methylobacterium sp.
  source 1..1482: mol_type = genomic DNA
cttgagagtttgatcctggctcagagcgaacgctggcggcaggcttaacacatgcaagtcgagcgggcatcttcggatgtcagcggcagacgggtgagtaacacgtgggaacgtacccttcggttcggaataacgctgggaaactagcgctaataccggatacgcccttttggggaaaggtttactgccgaaggatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccattcaagtcgggggtgaaagcctgtggctcaaccacagaattgccttcgatactgtttggcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcacctcagtagcgcagctaacgctttaagcattccgcctggggagtacggtcgcaagattaaaactcaaaggaattgacgggggcccgcacaagcggtggagcatgtggtttaattcgaagcaacgcgcagaaccttaccatcccttgacatggcatgttacccggagagattcggggtccacttcggtggcgtgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaaaccgtctcagttcagattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacctccttt

SEQ ID NO: 27 - DNA, 935 bp, Sphingomonas sp.
  source 1..935: mol_type = genomic DNA
cttgagagtttgatcctggctcagaacgaacgctggcggcatgcctaacacatgcaagtcgaacgatgctttcgggcatagtggcgcacgggtgcgtaacgcgtgggaatctgccctcaggttcggaataacagctggaaacggctgctaataccggatgatatcgcaagatcaaagatttatcgcctgaggatgagcccgcgttggattaggtagttggtggggtaaaggcctaccaagccgacgatccatagctggtctgagaggatgatcagccacactgggactgagacacggcccagactcctacgggaggcagcagtggggaatattggacaatgggcgcaagcctgatccagcaatgccgcgtgagtgatgaaggccctagggttgtaaagctcttttacccgggaagataatgactgtaccgggagaataagccccggctaactccgtgccagcagccgcggtaatacggagggggctagcgttgttcggaattactgggcgtaaagcgcacgtaggcggctttgtaagtcagaggtgaaagcctggagctcaactccagaactgcctttgagactgcatcgcttgaatccaggagaggtcagtggaattccgagtgtagaggtgaaattcgtagatattcggaagaacaccagtggcgaaggcggctgactggactggtattgacgctgaggtgcgaaagcgtggggagcaaacaggattagataccctggtagtccacgccgtaaacgatgataactagctgtccgggcacttggtgcttgggtggcgcagctaacgcattaagttatccgcctggggagtacggccgcaaggttaaaactcaaaggaattgacgggggcctgcacaagcggtggagcatgtggtttaattcgaagcaacgcgcagaaccttaccagcgtttgac

SEQ ID NO: 28 - DNA, 931 bp, Aureobasidium pullulans
  source 1..931: mol_type = genomic DNA
atagtcgggggcatcagtattcaattgtcagaggtgaaattcttggatttattgaagactaactactgcgaaagcatttgccaaggatgttttcattaatcagtgaacgaaagttaggggatcgaagacgatcagataccgtcgtagtcttaaccataaactatgccgactagggatcgggcgatgttatcattttgactcgctcggcaccttacgagaaatcaaagtctttgggttctggggggagtatggtcgcaaggctgaaacttaaagaaattgacggaagggcaccaccaggcgtggagcctgcggcttaatttgactcaacacggggaaactcaccaggtccagacacaataaggattgacagattgagagctctttcttgattttgtgggtggtggtgcatggccgttcttagttggtggagtgatttgtctgcttaattgcgataacgaacgagaccttaacctgctaaatagcccggcccgctttggcgggtcgccggcttcttagagggactatcggctcaagccgatggaagtttgaggcaataacaggtctgtgatgcccttagatgttctgggccgcacgcgcgctacactgacagagccaacgagttcatttccttgcccggaagggttgggtaatcttgttaaactctgtcgtgctggggatagagcattgcaattattgctcttcaacgaggaatgcctagtaagcgtacgtcatcagcgtgcgttgattacgtccctgccctttgtacacaccgcccgtcgctactaccgattgaatggctgagtgaggccttcggactggcccagggaggtcggcaacgaccacccagggccggaaagttggtcaaactccgtcatttagaggaagtaaaagtcgtaacaaggtttccgtaggtgaacctgcggaaggatca

SEQ ID NO: 29 - DNA, 1002 bp, Pseudoclavibacter helvolus
  source 1..1002: mol_type = genomic DNA
tacggagagtttgatcctggctcaggacgaacgctggcggcgtgcttaacacatgcaagtcgaacgatgaagcccagcttgctgggttgattagtggcgaacgggtgagtaacacgtgagcaacgtgcccataactctgggataacctccggaaacggtggctaatactggatatctaacacgatcgcatggtctgtgtttggaaagattttttggttatggatcggctcacggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggcattcgggttgtaaacctcttttagtagggaagaagcgaaagtgacggtacctgcagaaaaagcaccggctaactacgtgccagcagccgctgtaatacgtagggtgcaagcgttgtccggaattattgggcgtaaagagctcgtaggcggtttgtcgcgtctgctgtgaaatcccgaggctcaacctcgggtctgcagtgggtacgggcagactagagtgtggtaggggagattggaattcctggtgtagcggtggaatgcgcagatatcaggaggaacaccgatggcgaaggcagatctctgggccattactgacgctgaggagcgaaagcatggggagcgaacaggattagataccctggtagtccatgccgtaaacgttgggcgctagatgtggggaccattccacggtttccgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatataccggaaacgttcagaaatgttcgcc

SEQ ID NO: 30 - DNA, 1520 bp, Microbacterium testaceum
  source 1..1520: mol_type = genomic DNA
tacggagagtttgatcctggctcaggatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagacgtgatcgcatggtcgtgtttggaaagatttttcggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggaccattccacggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcgtaacaaggtagccgtaccggaaggtgcggctggatcacctccttt

SEQ ID NO: 31 - DNA, 941 bp, Sporisorium reilianum
  source 1..941: mol_type = genomic DNA
cagccgggggcattagtatttgcacgctagaggtgaaattcttggattgtgcaaagacttcctactgcgaaagcatttgccaagaatgttttcattaatcaagaacgaaggttagggtatcgaaaacgattagataccgttgtagtcttaacagtaaactatgccgactccgaatcggtcgatgctcatttcactggctcgatcggcgcggtacgagaaatcaaagtttttgggttctggggggagtatggtcgcaaggctgaaacttaaagaaattgacggaagggcaccaccaggagtggagcctgcggcttaatttgactcaacacgggaaaactcaccgggtccggacatagtaaggattgacagattgatggcgctttcatgattctatgggtggtggtgcatggccgttcttagttggtggagtgatttgtctggttaattccgataacgaacgagaccttgacctgctaaatagacgggttgacattttgttggccccttatgtcttcttagagggacaatcgaccgtctaggtgatggaggcaaaaggcaataacaggtctgtgatgcccttagatgttccgggctgcacgcgcgctacactgacagagacaacgagtggggccccttgtccgaaatgactgggtaaacttgtgaaactttgtcgtgctggggatggagctttgtaatttttgctcttcaacgaggaattcctagtaagcgcaagtcatcagcttgcgttgactacgtccctgccctttgtacacaccgcccgtcgctactaccgattgaatggcttagtgaggacttgggagagtacatcggggagccagcaatggcaccctgacggctcaaactcttacaaacttggtcatttagaggaagtaaaagtcgtaacaaggtatctgtaggtgaacctgcagatggatcatttc

SEQ ID NO: 32 - DNA, 1072 bp, Hafnia paralvei
  source 1..1072: mol_type = genomic DNA
actgagcattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 33 - DNA, 853 bp, Erwinia persicinus
  source 1..853: mol_type = genomic DNA
ggaggaaggcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccacggaattcggcagagatgccttagtgccttcgggaaccgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 34 - DNA, 1519 bp, Plantibacter flavus
  source 1..1519: mol_type = genomic DNA
tacggagagtttgatcctggctcaggacgaacgctggcggcgtgcttaacacatgcaagtcgaacgatgaagcccagcttgctgggtggattagtggcgaacgggtgagtaacacgtgagtaacctgcccttgactctgggataagcgttggaaacgacgtctaataccggatacgagcttccaccgcatggtgagttgctggaaagaattttggtcaaggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggacgacggccttcgggttgtaaacctcttttagcagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggtctgcagtgggtacgggcagactagagtgcggtaggggagattggaattcctggtgtagcggtggaatgcgcagatatcaggaggaacaccgatggcgaaggcagatctctgggccgctactgacgctgaggagcgaaagggtggggagcaaacaggcttagataccctggtagtccaccccgtaaacgttgggcgctagatgtggggaccattccacggtttccgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggacgacgtcaaatcatcatgccccttatgtcttgggcttcacgcatgctacaatggccagtacaaagggctgcaataccgtaaggtggagcgaatcccaaaaagctggtcccagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccagtggcctaaccgcaaggatggagctgtctaaggtgggatcggtaattaggactaagtcgtaacaaggtagccgtaccggaaggtgcggctggatcacctccttt

SEQ ID NO: 35 - DNA, 1536 bp, Pantoea ananatis
  source 1..1536: mol_type = genomic DNA
ttgaagagtttgatcatggctcagattgaacgctggcggcaggcctaacacatgcaagtcggacggtagcacagagagcttgctcttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatgaggttaataaccgcgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagcgaacttagcagagatgctttggtgccttcgggaacgctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcattactggggtgaagtcgtaacaaggtaaccgtaggggaacctgcggttggatcacctcctt

SEQ ID NO: 36 - DNA, 993 bp, Pantoea vagans
  source 1..993: mol_type = genomic DNA
ttgaagagtttgatcatggctcagattgaacgctggcggcaggcctaacacatgcaagtcggacggtagcacagagagcttgctcttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatgcggttaataaccgcgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatc

SEQ ID NO: 37 - DNA, 1532 bp, Pseudomonas rhodesiae
  source 1..1532: mol_type = genomic DNA
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacgaacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggccattacctaatacgtgatggttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggacggttaccacggtgtgattcatgactggggtgaagtcgtaacaaggtagccgtaggggaacctgcggctggatcacctcctt

SEQ ID NO: 38 - DNA, 1517 bp, Rhodococcus sp.
  source 1..1517: mol_type = genomic DNA
tacggagagtttgatcctggctcaggacgaacgctggcggcgtgcttaacacatgcaagtcgagcggtaaggcctttcggggtacacgagcggcgaacgggtgagtaacacgtgggtgatctgccctgcactctgggataagcttgggaaactgggtctaataccggatatgaccacagcatgcatgtgttgtggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcggaagcctgatgcagcgacgccgcgtgagggatgaaggccttcgggttgtaaacctctttcagcagggacgaagcgtgagtgacggtacctgcagaagaagcaccggctaactacgtgccagcagccgcggtaatacgtagggtgcgagcgttgtccggaattactgggcgtaaagagttcgtaggcggtttgtcgcgtcgtttgtgaaaacccggggctcaacttcgggcttgcaggcgatacgggcagacttgagtgtttcaggggagactggaattcctggtgtagcggtgaaatgcgcagatatcaggaggaacaccggtggcgaaggcgggtctctgggaaacaactgacgctgaggaacgaaagcgtgggtagcaaacaggattagataccctggtagtccacgccgtaaacggtgggcgctaggtgtgggttccttccacgggatctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatacaccggaaaaccgtagagatacggtcccccttgtggtcggtgtacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttacggggagggagccgtcgaaggtgggatcggcgattgggacgaagtcgtaacaaggtagccgtaccggaaggtgcggctggatcacctccttt

SEQ ID NO: 39 - DNA, 1482 bp, Agrobacterium tumefaciens
  source 1..1482: mol_type = genomic DNA
cttgagagtttgatcctggctcagaacgaacgctggcggcaggcttaacacatgcaagtcgaacgccccgcaaggggagtggcagacgggtgagtaacgcgtgggaatctaccgtgccctgcggaatagctccgggaaactggaattaataccgcatacgccctacgggggaaagatttatcggggtatgatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccttagggttgtaaagctctttcaccggagaagataatgacggtatccggagaagaagccccggctaacttcgtgccagcagccgcggtaatacgaagggggctagcgttgttcggaattactgggcgtaaagcgcacgtaggcggatatttaagtcaggggtgaaatcccagagctcaactctggaactgcctttgatactgggtatcttgagtatggaagaggtaagtggaattccgagtgtagaggtgaaattcgtagatattcggaggaacaccagtggcgaaggcggcttactggtccattactgacgctgaggtgcgaaagcgtggggagcaaacaggattagataccctggtagtccacgccgtaaacgatgaatgttagccgtcgggcagtatactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagctcttgacattcggggtttgggcagtggagacattgtccttcagttaggctggccccagaacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgagacagcgatgtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttttacccgaaggtagtgcgctaaccgcaaggaggcagctaaccacggtagggtcagcgactggggtgaagtcgtaacaaggtagccgtaggggaacctgcggctggatcacctccttt

SEQ ID NO: 40 - DNA, 903 bp, Pantoea sp.
  source 1..903: mol_type = genomic DNA
ttgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcgcgtgatggcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgt

SEQ ID NO: 41 - DNA, 1457 bp, Corynebacterium mucifaciens
  source 1..1457: mol_type = genomic DNA
gtggagagtttgatcctggctcaggatgaacgctggcggcgtgcttaacacatgcaagtcgaacggaaaggcccaagcttgcttgggtactcgagtggcgaacgggtgagtaacacgtgggtgatctgccctgcacttcgggataagcctgggaaactgggtctaataccggataggacgatggtttggatgccattgtggaaagttttttcggtgtgggatgagctcgcggcctatcagcttgttggtggggtaatggcctaccaaggcgtcgacgggtagccggcctgagagggtgtacggccacattgggactgagatacggcccagactcctacgggaggcagcagtggggaatattgcacaatgggcgcaagcctgatgcagcgacgccgcgtgggggatgacggccttcgggttgtaaactcctttcgctagggacgaagcgttttgtgacggtacctggagaagaagcaccggctaactacgtgccagcagccgcggtaatacgtagggtgcgagcgttgtccggaattactgggcgtaaagagctcgtaggtggtttgtcgcgtcgtttgtgtaagcccgcagcttaactgcgggactgcaggcgatacgggcataacttgagtgctgtaggggagactggaattcctggtgtagcggtggaatgcgcagatatcaggaggaacaccgatggcgaaggcaggtctctgggcagtaactgacgctgaggagcgaaagcatgggtagcgaacaggattagataccctggtagtccatgccgtaaacggtgggcgctaggtgtgagtcccttccacggggttcgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatacaccagatcgccgtagagatacggtttccctttgtggttggtgtacaggtggtgcatggttgtcgtcagctcgtgtcgtgagatgttgggttaagtcccgcaacgagcgcaacccttgtcttatgttgccagcacgtgatggtggggactcgtgagagactgccggggttaactcggaggaaggtggggatgacgtcaaatcatcatgccccttatgtccagggcttcacacatgctacaatggtcggtacaacgcgcatgcgagcctgtgagggtgagcgaatcgctgtgaaagccggtcgtagttcggattggggtctgcaactcgaccccatgaagtcggagtcgctagtaatcgcagatcagcaacgctgcggtgaatacgttcccgggccttgtacacaccgcccgtcacaccatgggagtgggttgcaaaagaagtaggtagcttaaccttcgggagggcgcttaccactttgtgat

SEQ ID NO: 42 - DNA, 1532 bp, Pseudomonas lundensis
  source 1..1532: mol_type = genomic DNA
tgaagagtttgatcatggctcagattgaacgctggcggcaggcctaacacatgcaagtcgagcggtagagaggtgcttgcacctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggaaccttgagttcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccctcgggaggacggttaccacggtgtgattcatgactggggtgaagtcgtaacaaggtagccgtaggggaacctgcggctggatcacctcctt

SEQ ID NO: 43 - DNA, 1525 bp, Janthinobacterium sp.
  source 1..1525: mol_type = genomic DNA
ctgagtttgatcctggctcagattgaacgctggcggcatgccttacacatgcaagtcgaacggcagcacggagcttgctctggtggcgagtggcgaacgggtgagtaatatatcggaacgtaccctggagtgggggataacgtagcgaaagttacgctaataccgcatacgatctaaggatgaaagtgggggatcgcaagacctcatgctcgtggagcggccgatatctgattagctagttggtagggtaaaagcctaccaaggcatcgatcagtagctggtctgagaggacgaccagccacactggaactgagacacggtccagactcctacgggaggcagcagtggggaattttggacaatgggcgaaagcctgatccagcaatgccgcgtgagtgaagaaggccttcgggttgtaaagctcttttgtcagggaagaaacggtgagagctaatatctcttgctaatgacggtacctgaagaataagcaccggctaactacgtgccagcagccgcggtaatacgtagggtgcaagcgttaatcggaattactgggcgtaaagcgtgcgcaggcggttttgtaagtctgatgtgaaatccccgggctcaacctgggaattgcattggagactgcaaggctagaatctggcagaggggggtagaattccacgtgtagcagtgaaatgcgtagatatgtggaggaacaccgatggcgaaggcagccccctgggtcaagattgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatggctggaatccttgagagatcagggagtgctcgaaagagaaccagtacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagcgccgccaacccgcgagggggagctaatcgcagaaagtgtatcgtagtccggattgtagtctgcaactcgactgcatgaagttggaatcgctagtaatcgcggatcagcatgtcgcggtgaatacgttcccgggtcttgtacacaccgcccgtcacaccatgggagcgggttttaccagaagtaggtagcttaaccgtaaggagggcgcttaccacggtaggattcgtgactggggtgaagtcgtaacaaggtagccgtatcggaaggtgcggctggatcacctccttt

SEQ ID NO: 44 - DNA, 1497 bp, Herbaspirillum sp.
  source 1..1497: mol_type = genomic DNA
tggcggcatgccttacacatgcaagtcgaacggcagcataggagcttgctcctgatggcgagtggcgaacgggtgagtaatatatcggaacgtgccctagagtgggggataactagtcgaaagactagctaataccgcatacgatctacggatgaaagtgggggatcgcaagacctcatgctcctggagcggccgatatctgattagctagttggtggggtaaaagctcaccaaggcgacgatcagtagctggtctgagaggacgaccagccacactgggactgagacacggcccagactcctacgggaggcagcagtggggaattttggacaatgggggcaaccctgatccagcaatgccgcgtgagtgaagaaggccttcgggttgtaaagctcttttgtcagggaagaaacggttctggataatacctaggactaatgacggtacctgaagaataagcaccggctaactacgtgccagcagccgcggtaatacgtagggtgcaagcgttaatcggaattactgggcgtaaagcgtgcgcaggcggttgtgtaagtcagatgtgaaatccccgggctcaacctgggaattgcatttgagactgcacggctagagtgtgtcagaggggggtagaattccacgtgtagcagtgaaatgcgtagatatgtggaggaataccgatggcgaaggcagccccctgggataacactgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatggatggaatcccgaagagatttgggagtgctcgaaagagaaccatcacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggattggagtctgcaactcgactccatgaagttggaatcgctagtaatcgcggatcagcatgtcgcggtgaatacgttcccgggtcttgtacacaccgcccgtcacaccatgggagcgggttttaccagaagtgggtagcctaaccgcaaggagggcgctcaccacggtaggattcgtgactggggtgaagtcgtaacaaggtagccgtatcggaaggtgcggctggatcacctccttt

SEQ ID NO: 45 - DNA, 1522 bp, Sanguibacter keddieii
  source 1..1522: mol_type = genomic DNA
tacggagagtttgatcctggctcaggacgaacgctggcggcgtgcttaacacatgcaagtcgaacggtgacgctagagcttgctctggttgatcagtggcgaacgggtgagtaacacgtgagtaacctgcccttgactctgggataactccgggaaaccggggctaataccggatacgagacgcgaccgcatggtcggcgtctggaaagtttttcggtcaaggatggactcgcggcctatcagcttgttggtgaggtaatggctcaccaaggcgtcgacgggtagccggcctgagagggcgaccggccacactgggactgagacacggcccagactcctacgggaggcagcagtggggaatattgcacaatgggcgaaagcctgatgcagcgacgccgcgtgagggatgaaggccttcgggttgtaaacctctttcagtagggaagaagcgaaagtgacggtacctgcagaagaagcgccggctaactacgtgccagcagccgcggtaatacgtagggcgcaagcgttgtccggaattattgggcgtaaagagctcgtaggcggtttgtcgcgtctggtgtgaaaactcaaggctcaaccttgagcttgcatcgggtacgggcagactagagtgtggtaggggtgactggaattcctggtgtagcggtggaatgcgcagatatcaggaggaacaccgatggcgaaggcaggtcactgggccactactgacgctgaggagcgaaagcatggggagcgaacaggattagataccctggtagtccatgccgtaaacgttgggcactaggtgtggggctcattccacgagttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatacaccggaatcatgcagagatgtgtgcgtcttcggactggtgtacaggtggtgcatggttgtcgtcagctcgtgtcgtgagatgttgggttaagtcccgcaacgagcgcaaccctcgtcctatgttgccagcacgttatggtggggactcataggagactgccggggtcaactcggaggaaggtggggatgacgtcaaatcatcatgccccttatgtcttgggcttcacgcatgctacaatggccggtacaaagggctgcgataccgcgaggtggagcgaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaaccccttgtgggatggagccgtcgaaggtgggattggcgattgggactaagtcgtaacaaggtagccgtaccggaaggtgcggctggatcacctccttt

SEQ ID NO: 46 - DNA, 831 bp, Pantoea agglomerans
  source 1..831: mol_type = genomic DNA
ttgaagagtttgatcatggctcagattgaacgctggcggcaggcctaacacatgcaagtcggacggtagcacagaggagcttgctccttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgacagggttaataaccctgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggctttagtcttgtagagtggggtagaattccaggtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgtt

SEQ ID NO: 47 - DNA, 960 bp, Cronobacter dublinensis
  source 1..960: mol_type = genomic DNA
agggtgcaagcgttaatcggaattactgggcgtaaagcgcgcgtaggtggtttgttaagttgaatgtgaaatccccgggctcaacctgggaactgcatttgaaactggcaagctagagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgcaacgagcgcaacccttgtcctgtgttgccagcgcgtaatggcggggactcgcaggagactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagt

SEQ ID NO: 48 - DNA, 1548 bp, Bacillus paralicheniformis
  source 1..1548: mol_type = genomic DNA
catggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgcttgattgaaccgcatggttcaattataaaaggtggcttttagctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacctccttt

SEQ ID NO: 49 - DNA, 1551 bp, Bacillus gibsonii
  source 1..1551: mol_type = genomic DNA
tatggagagtttgatcctggctcaggacgaacgctggcggcgtgcctaatacatgcaagtcgagcggacgtttttgaagcttgcttcaaaaacgttagcggcggacgggtgagtaacacgtgggcaacctgccttatcgactgggataactccgggaaaccggggctaataccggataatatctagcacctcctggtgcaagattaaaagagggccttcgggctctcacggtgagatgggcccgcggcgcattagctagttggagaggtaatggctccccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaagggtttcggctcgtaaagctctgttatgagggaagaacacgtaccgttcgaatagggcggtaccttgacggtacctcatcagaaagccacggctaactacgtgccagcagccgcggtaatacgtaggtggcaagcgttgtccggaattattgggcgtaaagcgcgcgcaggcggccttttaagtctgatgtgaaatcttgcggctcaaccgcaagcggtcattggaaactgggaggcttgagtacagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacgggggcccgcacaagcagtggagcatgtggtttaattcgaagcaacgcgaagaaccttaccaggtcttgacatcctttgaccactctggagacagagcttccccttcgggggcaaagtgacaggtggtgcatggttgtcgtcagctcgtgtcgtgagatgttgggttaagtcccgcaacgagcgcaacccttgaccttagttgccagcatttagttgggcactctaaggtgactgccggtgacaaaccggaggaaggtggggatgacgtcaaatcatcatgccccttatgacctgggctacacacgtgctacaatggatggtacaaagggttgcgaagccgcgaggtgaagccaatcccataaagccattctcagttcggattgtaggctgcaactcgcctgcatgaagctggaattgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacctccttt

SEQ ID NO: 50 - DNA, 1536 bp, Enterobacter sp.
  source 1..1536: mol_type = genomic DNA
ttgaagagtttgatcatggctcagattgaacgctggcggcaggcctaacacatgcaagtcgaacggtagcacagagagcttgctcttgggtgacgagtggcggacgggtgagtaatgtctgggaaactgcccgatggagggggataactactggaaacggtagctaataccgcataacgtcgcaagaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattgtggttaataaccgcagtgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttagcagagatgctttagtgccttcgggaaccgtgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 51 - DNA, 1537 bp, Klebsiella aerogenes
  source 1..1537: mol_type = genomic DNA
ttgaagagtttgatcatggctcagattgaacgctggcggcaggcctaacacatgcaagtcgagcggtagcacagggagcttgctcctgggtgacgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttggtgattgacgttactcgcagaagaagcaccggctaactccgtgccagcagccgcggtaatacggggggtgcaagcgttaatcggaattactgggcgtaaagcgcacgcaggcggtttgtcaagtcggatgtgaaatccccgggctcaacctgggaactgcattcgaaacgggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccagtgacaaactggaggaaggtggggatgacgtcaagtcatcatggcccttacgagtagggctacacacgtgctacaatggcatatacaaagagaagcgacctcgcgagagcaagcggacctcacaaagtatgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 52 - DNA, 1517 bp, Arthrobacter sp.
  source 1..1517: mol_type = genomic DNA
acggagagtttgatcctggctcaggatgaacgctggcggcgtgcttaacacatgcaagtcgaacgatgatcccagcttgctgggggattagtggcgaacgggtgagtaacacgtgagtaacctgcccttgactctgggataagcctgggaaactgggtctaataccggatatgactgtctgacgcatgtcaggtggtggaaagcttttgtggttttggatggactcgcggcctatcagcttgttggtggggtaatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcgccggctaactacgtgccagcagccgcggtaatacgtagggcgcaagcgttatccggaattattgggcgtaaagagctcgtaggcggtttgtcgcgtctgctgtgaaagaccggggctcaactccggttctgcagtgggtacgggcagactagagtgcagtaggggagactggaattcctggtgtagcggtgaaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaatacctggaaacaggtgccccgcttgcggtcggtttacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccggcgattgggactaagtcgtaacaaggtagccgtaccggaaggtgcggctggatcacctccttt

SEQ ID NO: 53 - DNA, 1406 bp, Pseudomonas fragi
  source 1..1406: mol_type = genomic DNA
tgaagagtttgatcatggctcagattgaacgctggcggcaggcctaacacatgcaagtcgagcggtagagagaagcttgcttctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatg

SEQ ID NO: 54 - DNA, 1136 bp, Methylobacterium adhaesivum
  source 1..1136: mol_type = genomic DNA
cttgagagtttgatcctggctcagagcgaacgctggcggcaggcttaacacatgcaagtcgagcgggcaccttcgggtgtcagcggcagacgggtgagtaacacgtgggaacgtacccttcggttcggaataacgctgggaaactagcgctaataccggatacgcccttttggggaaaggtttactgccgaaggatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccattcaagtcgggggtgaaagcctgtggctcaaccacagaattgccttcgatactgtttggcttgagtttggtagaggttggtggaactgcgagtgtagaggtgaaattcgtagatattcgcaagaacaccagtggcgaaggcggccaactggaccaatactgacgctgaggcgcgaaagcgtggggagcaaacaggattagataccctggtagtccacgccgtaaacgatgaatgctagctgttggggtgcttgcacctcagtagcgcagctaacgctttaagcattccgcctggggagtacggtcgcaagattaaaactcaaaggaattgacgggggcccgcacaagcggtggagcatgtggtttaattcgaagcaacgcgcagaaccttaccatcccttgacatgtcgtgccatccggagagatccggggttcccttcggggacgcgaacacaggtgctgcatggctgtcgtcagctcgtgtcgtgagatgttgggttaagtcccgcaacgagcgcaacccacgtccttagttgccatcatttagttgggcactctagggagactgccggtgataagccgcgaggaaggtgtggatgacgtc

SEQ ID NO: 55 - DNA, 1374 bp, Bacillus megaterium
  source 1..1374: mol_type = genomic DNA
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgct

SEQ ID NO: 56 - DNA, 1554 bp, Paenibacillus lautus
  source 1..1554: mol_type = genomic DNA
attggagagtttgatcctggctcaggacgaacgctggcggcgtgcctaatacatgcaagtcgagcggacctgatggagtgcttgcactcctgatggttagcggcggacgggtgagtaacacgtaggcaacctgccctcaagactgggataactaccggaaacggtagctaataccggataatttatttcacagcattgtggaataatgaaagacggagcaatctgtcacttggggatgggcctgcggcgcattagctagttggtggggtaacggctcaccaaggcgacgatgcgtagccgacctgagagggtgaacggccacactgggactgagacacggcccagactcctacgggaggcagcagtagggaatcttccgcaatgggcgaaagcctgacggagcaacgccgcgtgagtgatgaaggttttcggatcgtaaagctctgttgccaaggaagaacgtcttctagagtaactgctaggagagtgacggtacttgagaagaaagccccggctaactacgtgccagcagccgcggtaatacgtagggggcaagcgttgtccggaattattgggcgtaaagcgcgcgcaggcggttctttaagtctggtgtttaaacccgaggctcaacttcgggtcgcactggaaactggggaacttgagtgcagaagaggagagtggaattccacgtgtagcggtgaaatgcgtagatatgtggaggaacaccagtggcgaaggcgactctctgggctgtaactgacgctgaggcgcgaaagcgtggggagcaaacaggattagataccctggtagtccacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaagtcttgacatccctctgaatcctctagagatagaggcggccttcgggacagaggtgacaggtggtgcatggttgtcgtcagctcgtgtcgtgagatgttgggttaagtcccgcaacgagcgcaacccttgattttagttgccagcacatcatggtgggcactctagaatgactgccggtgacaaaccggaggaaggcggggatgacgtcaaatcatcatgccccttatgacttgggctacacacgtactacaatggctggtacaacgggaagcgaagccgcgaggtggagccaatcctataaaagccagtctcagttcggattgcaggctgcaactcgcctgcatgaagtcggaattgctagtaatcgcggatcagcatgccgcggtgaatacgttcccgggtcttgtacacaccgcccgtcacaccacgagagtttacaacacccgaagtcggtggggtaacccgcaagggagccagccgccgaaggtggggtagatgattggggtgaagtcgtaacaaggtagccgtatcggaaggtgcggctggatcacctccttt

SEQ ID NO: 57 - DNA, 1550 bp, Bacillus mycoides
  source 1..1550: mol_type = genomic DNA
attggagagtttgatcctggctcaggatgaacgctggcggcgtgcctaatacatgcaagtcgagcgaatggattaagagcttgctcttatgaagttagcggcggacgggtgagtaacacgtgggtaacctgcccataagactgggataactccgggaaaccggggctaataccggataacattttgcaccgcatggtgcgaaattcaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaacctttttggagccagccgcctaaggtgggacagatgattggggtgaagtcgtaacaaggtagccgtatcggaaggtgcggctggatcacctccttt

SEQ ID NO: 58 - DNA, 1060 bp, Janthinobacterium svalbardensis
  source 1..1060: mol_type = genomic DNA
aatgacggtacctgaagaataagcaccggctaactacgtgccagcagccgcggtaatacgtagggtgcaagcgttaatcggaattactgggcgtaaagcgtgcgcaggcggttttgtaagtctgatgtgaaatccccgggctcaacctgggaattgcattggagactgcaaggctagaatctggcagaggggggtagaattccacgtgtagcagtgaaatgcgtagatatgtggaggaacaccgatggcgaaggcagccccctgggtcaagattgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatggctggaatcctcgagagattggggagtgctcgaaagagaaccagtacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagcgccgccaacccgcgagggggagctaatcgcagaaagtgtatcgtagtccggattgtagtctgcaactcgactgcatgaagttggaatcgctagtaatcgcggatcagcatgtcgcggtgaatacgttcccgggtcttgtacacaccgcccgtcacaccatgggagcgggttttaccagaagtaggtagcttaaccgtaaggagggcgcttaccacggtaggattcgtgactggggtgaagtcgtaacaaggtagccgtatcggaaggtgcggctggatcacctccttt

SEQ ID NO: 59 - DNA, 1538 bp, Kosakonia cowanii
  source 1..1538: mol_type = genomic DNA
ttgaagagtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatgcggttaataaccgcgtcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaacttggcagagatgccttggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcgcatacaaagagaagcgatctcgcgagagccagcggacctcataaagtgcgtcgtagtccggattggagtctgcaactcgactccatgaagtcggaatcgctagtaatcgtgaatcagaatgtcacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 60 - DNA, 1547 bp, Bacillus simplex
  source 1..1547: mol_type = genomic DNA
tcggagagtttgatcctggctcaggacgaacgctggcggcgtgcctaatacatgcaagtcgagcgaatcgatgggagcttgctccctgagattagcggcggacgggtgagtaacacgtgggcaacctgcctataagactgggataacttcgggaaaccggagctaataccggatacgttcttttctcgcatgagagaagatggaaagacggttttgctgtcacttatagatgggcccgcggcgcattagctagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgaagaaggccttcgggtcgtaaagttctgttgttagggaagaacaagtaccagagtaactgctggtaccttgacggtacctaaccagaaagccacggctaactacgtgccagcagccgcggtaatacgtaggtggcaagcgttgtccggaattattgggcgtaaagcgcgcgcaggtggttccttaagtctgatgtgaaagcccacggctcaaccgtggagggtcattggaaactggggaacttgagtgcagaagaggaaagtggaattccaagtgtagcggtgaaatgcgtagagatttggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaaacctgcgaaggtaagcgaatcccataaagccattctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtgaggtaacctttatggagccagccgcctaaggtgggacagatgattggggtgaagtcgtaacaaggtagccgtatcggaaggtgcggctggatcacctccttt

SEQ ID NO: 61 - DNA, 962 bp, Lelliottia sp.
  source 1..962: mol_type = genomic DNA
ggaaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttagcagagatgctttagtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 62 - DNA, 876 bp, Erwinia sp.
  source 1..876: mol_type = genomic DNA
tggctcagattgaacgctggcggcaggcctaacacatgcaagtcgaacggtagcacagaggagcttgctccttgggtgacgagtggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagtggggaggaaggcgttaaggttaataaccttggcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacgg

SEQ ID NO: 63 - DNA, 1532 bp, Pseudomonas azotoformans
  source 1..1532: mol_type = genomic DNA
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgttcttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 64 - DNA, 1024 bp, Hanseniaspora uvarum
  source 1..1024: mol_type = genomic DNA
gctcgagttcttgtttagatcttttacaataatgtgtatctttactgaagatgtgcgcttaattgcgctgcttctttagagtgtcgcagtgaaagtagtcttgcttgaatctcagtcaacgctacacacattggagtttttttactttaatttaattctttctgctttgaatcgaaaggttcaaggcaaaaaacaaacacaaacaattttattttattataattttttaaactaaaccaaaattcctaacggaaattttaaaataatttaaaactttcaacaacggatctcttggttctcgcatcgatgaagaacgtagcgaattgcgataagtaatgtgaattgcagatactcgtgaatcattgaatttttgaacgcacattgcgcccttgagcattctcaggggcatgcctgtttgagcgtcatttccttctcaaaagataatttattattttttggttgtgggcgatactcagggttagcttgaaattggagactgtttcagtcttttttaattcaacacttagcttctttggagacgctgttctcgctgtgatgtatttatggatttattcgttttactttacaagggaaatggtaacgtaccttaggcaaagggttgcttttaatattcatcaagtttgacctcaaatcaggtaggattacccgctgaacttaagcatatcaataagcggaggaaaagaaaccaactgggattaccttagtaacggcgagtgaagcggtaaaagctcaaatttgaaatctggtactttcagtgcccgagttgtaatttgtagaatttgtctttgattaggtccttgtctatgttccttggnancaggacgtcatagagggtgagaatcccgtttggcgaggataccttttctctgtaagactttttcgaanantcgagttgtttgggaatgcagctcaaagtgggtggtaaanttccatctaaagctaaatnttggcgagagaccgatagcgaacnagtacagtgatggaaagatgaaaaagaantttn

SEQ ID NO: 65 - DNA, 1550 bp, Bacillus sp.
  source 1..1550: mol_type = genomic DNA
attggagagtttgatcctggctcaggatgaacgctggcggcgtgcctaatacatgcaagtcgagcgaatggattaagagcttgctcttatgaagttagcggcggacgggtgagtaacacgtgggtaacctgcccataagactgggataactccgggaaaccggggctaataccggataacattttgaactgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaacctttttggagccagccgcctaaggtgggacagatgattggggtgaagtcgtaacaaggtagccgtatcggaaggtgcggctggatcacctccttt

SEQ ID NO: 66 - DNA, 1034 bp, Hanseniaspora occidentalis
  source 1..1034: mol_type = genomic DNA
gattttttggggttgcttcgaacttgcagacagagtgtcgagacttgtgagcctgcgcttaattgcgcggcctagagtcgagtgcttgttattggctgcgagggacgagtgccttttgaaaaaatccattacacactgtgaagattttttttcatacattttacttctttggggctttcgagctccaaaggctataaacacaaaccaaacttttttttttattatttgttaatcaagaaattttcttattgaaattaaatattttaaaactttcaacaacggatctcttggttctcgcatcgatgaagaacgtagcgaattgcgataagtaatgtgaattgcagattctcgtgaatcattgaatttttgaacgcacattgcgccctctggtattccagggggcatgcctgtttgagcgtcatttccttctcaaaatctcgattttggttgtgagtgatactctgttacagggttaacttgaaagtgctattgccctagctactcttttttttacttgctaagaaaaagatttttggataatttcaatgtatttaggtatttataccgactttcattggatgctgagagtcttgtctaagcgcttttgtgagattgagcagaagggattaacagtattcataaagtttgacctcaaatcaggtaggattacccgctgaacttaagcatatcaataagcggaggaaaagaaaccaaccgggattgcctcagtaacggcgagtgaagcggcaaaagctcaaatttgaaatctggcactttcagtgtccgagttgtaatttgtagaagtagttttgggactggtccttatctatgtttcttggaacaggacgtcatagagggtgagancccgtatgatgaggcccccagtcctttgtaaaacgctncgaagagtcgagttgtttgggaatgcagctctaagtgggtngnaattnntctaaagctaaatnnnnnnnanacnntngcganagtacngtgatgnngatgannactttgaaananantgaaaagtacgtgaa

SEQ ID NO: 67 - DNA, 980 bp, Bacillus sp.
  source 1..980: mol_type = genomic DNA
tcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaccctagagatagggcttcccttcgggg

SEQ ID NO: 68 - DNA, 680 bp, Bacillus atrophaeus
  source 1..680: mol_type = genomic DNA
tgcagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaa

SEQ ID NO: 69 - DNA, 644 bp, Bacillus sp.
  source 1..644: mol_type = genomic DNA
tgcagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtg

SEQ ID NO: 70 - DNA, 787 bp, Bacillus subtilis
  source 1..787: mol_type = genomic DNA
tgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgangagcgaaagcgtggggagcgaacaggattagataccctggtagtccacgccgtaaacgatgagtgctaagtgtta

SEQ ID NO: 71 - DNA, 914 bp, Rhodotorula sp.
  source 1..914: mol_type = genomic DNA
ttacttggagtccgaactctcactttttaaccctgtgcatctgttaattggaatagtagctcttcggagtgaaccaccattcacttataaaacacaaagtctatgaatgtatacaaatttataacaaaacaaaactttcaacaacggatctcttggctctcgcatcgatgaagaacgcagcgaaatgcgatacgtaatgtgaattgcagaattcagtgaatcatcgaatctttgaacgcaccttgcgctccttggtattccgaggagcatgcctgtttgagtgtcatgaaatcttcaacccacctctttcttagtgaatctggtggtgcttggtttctgagcgctgctctgcttcggcttagctcgttcgtaatgcattagcatccgcaaccgaacttcggattgacttggcgtaatagactattcgctgaggattctagtttactagagccgagttgggttaaaggaagctcctaatcctaaagtctattttttgattagatctcaaatcaggtaggactacccgctgaacttaagcatatcaataagcggaggaaaagaaactaacaaggattcccctagtagcggcgagcgaagcgggaagagctcaaatttataatctggcaccttcggtgtccgagttgtaatctctagaagtgttttccgcgttggaccgcacacaagtctgttggaatacagcggcatagtggtgaaacccccgtatatggtgcggacgcccagcgctttgtgatacactttcaatgagtcgagttgtttgggaatgcagctcaaattgggtggtaaattccatctaaagctaaatattggcgagagaccgatagcgaacaagtaccgtgagggaaagatgaaaagcactttggaaagagagttaacagtacgtgaaattgttggaa

SEQ ID NO: 72 - DNA, 1547 bp, Bacillus zhangzhouensis
  source 1..1547: mol_type = genomic DNA
ttcggagagtttgatcctggctcaggacgaacgctggcggcgtgcctaatacatgcaagtcgagcggacagaagggagcttgctcccggatgttagcggcggacgggtgagtaacacgtgggtaacctgcctgtaagactgggataactccgggaaaccggagctaataccggatagttccttgaaccgcatggttcaaggatgaaagacggtttcggctgtcacttacagatggacccgcggcgcattagctagttggtggggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgcgagagtaactgctcgcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggacagaacaaagggctgcgagaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcgtaacaaggtagccgtatcggaaggtgcggctggatcacctccttt

SEQ ID NO: 73 - DNA, 841 bp, Bacillus clausii
  source 1..841: mol_type = genomic DNA
aacggagagtttgatcctggctcaggacgaacgctggcggcgtgcctaatacatgcaagtcgagcggacagaagggagcttgctcccggacgttagcggcggacgggtgagtaacacgtgggcaacctgccccttagactgggataactccgggaaaccggagctaataccggataatccctttctccacctggagagagggtgaaagatggcttcggctatcactaagggatgggcccgcggcgcattagctagttggtaaggtaacggcttaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgaggaaggccttcgggtcgtaaagctctgttgtgagggaagaagcggtgccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttag

SEQ ID NO: 74 - DNA, 1105 bp, Bacillus coagulans
  source 1..1105: mol_type = genomic DNA
gcctaatacatgcaagtcgtgcggaccttttaaaagcttgcttttaaaaggttagcggcgaacgggtgagtaacacgtgggcaacctgcctgtaagatcgggataatgccgggaaaccggggctaataccggatagttttttcctccgcatggaggaaaaaggaaagacggcttcggctgtcacttacagatgggcccgcggcgcattagcttgttggtggggtaacggctcaccaaggcaacgatgcgtagccgacctgagagggtgatcggccacattgggactgagacacggcccaaactcctacgggaggcagcagtagggaatcttccgcaatggacgaaagtctgacggagcaacgccgcgtgagtgaagaaggccttcgggtcgtaaaactctgttgccggggaagaacaagtgccgttcgaacagggcggcgccttgacggtacccggccagaaagccacggctaactacgtgccagcagccgcggtaatacgtaggtggcaagcgttgtccggaattattgggcgtaaagcgcgcgcaggcggcttcttaagtctgatgtgaaatcttgcggctcaaccgcaagcggtcattggaaactgggaggcttgagtgcagaagaggagagtggaattccacgtgtagcggtgaaatgcgtagagatgtggaggaacaccagtggcgaaggcggctctctggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacctccctggagacagggccttccccttcgggggacagagtgacaggtggtgcatggttgtcgtcagctcgtgtcgtgagatgttgggttaagtcccgcaacgagcgcaacccttgaccttagttgccagcattcag

SEQ ID NO: 75 - DNA, 1515 bp, Pseudomonas gessardii
  source 1..1515: mol_type = genomic DNA
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggttcgttaagttggatgtgaaagccccgggctcaacctgggaactgcattcaaaactgacgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggatgggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaacgggaggacggttaccacggtgtgattcatgactggggtgaagtcgtaacaaggtagccgtaggggaacctgcggctggatcacctcctt

SEQ ID NO: 76 - DNA, 1479 bp, Ochrobactrum sp.
  source 1..1479: mol_type = genomic DNA
cttgagagtttgatcctggctcagaacgaacgctggcggcaggcttaacacatgcaagtcgagcgccccgcaaggggagcggcagacgggtgagtaacgcgtgggaatctaccttttgctacggaacaacagttggaaacgactgctaataccgtatgtgcccttcgggggaaagatttatcggcaaaggatgagcccgcgttggattagctagttggtgaggtaaaggctcaccaaggcgacgatccatagctggtctgagaggatgatcagccacactgggactgagacacggcccagactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggatttactgggcgtaaagcgcacgtaggcggatttttaagtcaggggtgaaatcccggggctcaaccccggaactgcctttgatactggaagtcttgagtatggtagaggtgagtggaattccgagtgtagaggtgaaattcgtagatattcggaggaacaccagtggcgaaggcggctcactggaccattactgacgctgaggtgcgaaagcgtggggagcaaacaggattagataccctggtagtccacgccgtaaacgatgaatgttagccgtcggggggtttacctttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacataccggtcgcggacacagagatgtgtctttcagttcggctggaccggatacaggtgctgcatggctgtcgtcagctcgtgtcgtgagatgttgggttaagtcccgcaacgagcgcaaccctcgcctttagttgccagcatttagttgggcactctaaagggactgccagtgataagctggaggaaggtggggatgacgtcaagtcctcatggcccttacgggctgggctacacacgtgctacaatggtggtgacagtgggcagcaagcacgcgagtgtgagctaatctccaaaagccatctcagttcggattgcactctgcaactcgagtgcatgaagttggaatcgctagtaatcgcggatcagcatgccgcggtgaatacgttcccgggccttgtacacaccgcccgtcacaccatgggagttggttttacccgaaggcactgtgctaaccgcaaggaggcaggtgaccacggtagggtcagcgactggggtgaagtcgtaacaaggtagccgtaggggaacctgcggctggatcacctccttt

SEQ ID NO: 77 - DNA, 1549 bp, Bacillus aryabhattai
  source 1..1549: mol_type = genomic DNA
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctccttt

SEQ ID NO: 78 - DNA, 1537 bp, Erwinia rhapontici
  source 1..1537: mol_type = genomic DNA
ttgaagagtttgatcatggctcagattgaacgctggcggcaggcctaacacatgcaagtcgaacggtagcacagagagcttgctcttgggtgacgagtggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagtggggaggaaggcgatgaagttaatagcttcgtcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccacggaattcggcagagatgccttagtgccttcgggaaccgtgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 79 - DNA, 1532 bp, Pseudomonas fragi
  source 1..1532: mol_type = genomic DNA
tgaagagtttgatcatggctcagattgaacgctggcggcaggcctaacacatgcaagtcgagcggtagagagaagcttgcttctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgactgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 80 - DNA, 1136 bp, Methylobacterium adhaesivum; identical to SEQ ID NO: 54
  source 1..1136: mol_type = genomic DNA

SEQ ID NO: 81 - DNA, 1547 bp, Bacillus clausii
  source 1..1547: mol_type = genomic DNA
aacggagagtttgatcctggctcaggacgaacgctggcggcgtgcctaatacatgcaagtcgagcggacagaagggagcttgctcccggacgttagcggcggacgggtgagtaacacgtgggcaacctgccccttagactgggataactccgggaaaccggagctaataccggataatccctttctccacctggagagagggtgaaagatggcttcggctatcactaggggatgggcccgcggcgcattagctagttggtaaggtaacggcttaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgaggaaggctttcgggtcgtaaagctctgttgtgagggaagaagcggtaccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacggggacccgcacaagcagtggagcatgtggtttaattcgaagcaacgcgaagaaccttaccaggtcttgacatcctttgaccacccaagagattgggcttccccttcgggggcaaagtgacaggtggtgcatggttgtcgtcagctcgtgtcgtgagatgttgggttaagtcccgcaacgagcgcaacccttgatcttagttgccagcattgagttgggcactctaaggtgactgccggtgacaaaccggaggaaggtggggatgacgtcaaatcatcatgccccttatgacctgggctacacacgtgctacaatggatggtacaaagggcagcgaaaccgcgaggtgaagccaatcccataaagccattctcagttcggattgcaggctgcaactcgcctgcatgaagccggaattgctagtaatcgcggatcagcatgccgcggtgaatacgttcccgggtcttgtacacaccgcccgtcacaccacgagagtttgtaacacccgaagtcggtgaggcaaccttttggagccagccgcctaaggtgggacaaatgattggggtgaagtcgtaacaaggtagccgtatcggaaggtgcggctggatcacctccttt

SEQ ID NO: 82 - DNA, 1547 bp, Bacillus clausii
  source 1..1547: mol_type = genomic DNA
aacggagagtttgatcctggctcaggacgaacgctggcggcgtgcctaatacatgcaagtcgagcggacagaagggagcttgctcccggacgttagcggcggacgggtgagtaacacgtgggcaacctgccccttagactgggataactccgggaaaccggagctaataccggataatccctttctccacctggagagagggtgaaagatggcttcggctatcactaagggatgggcccgcggcgcattagctagttggtaaggtaacggcttaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgaggaaggccttcgggtcgtaaagctctgttgtgagggaagaagcggtaccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacggggacccgcacaagcagtggagcatgtggtttaattcgaagcaacgcgaagaaccttaccaggtcttgacatcctttgaccacccaagagattgggcttccccttcgggggcaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggcagcgaaaccgcgaggtgaagccaatcccataaagccattctcagttcggattgcaggctgcaactcgcctgcatgaagccggaattgctagtaatcgcggatcagcatgccgcggtgaatacgttcccgggtcttgtacacaccgcccgtcacaccacgagagtttgtaacacccgaagtcggtgaggcaaccttttggagccagccgcctaaggtgggacaaatgattggggtgaagtcgtaacaaggtagccgtatcggaaggtgcggctggatcacctccttt

SEQ ID NO: 83 - DNA, 1548 bp, Bacillus clausii
  source 1..1548: mol_type = genomic DNA
acggagagtttgatcctggctcaggacgaacgctggcggcgtgcctaatacatgcaagtcgagcggagtttcaagaagcttgctttttgaaacttagcggcggacgggtgagtaacacgtgggcaacctgccccttagactgggataactccgggaaaccggagctaataccggataatccctttctccacctggagagagggtgaaagatggcttcggctatcactaagggatgggcccgcggcgcattagctagttggtaaggtaacggcttaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgaggaaggccttcgggtcgtaaagctctgttgtgagggaagaagcggtaccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacggggacccgcacaagcagtggagcatgtggtttaattcgaagcaacgcgaagaaccttaccaggtcttgacatcctttgaccacccaagagattgggcttccccttcgggggcaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggcagcgaagccgcgaggtgaagccaatcccataaagccattctcagttcggattgcaggctgcaactcgcctgcatgaagccggaattgctagtaatcgcggatcagcatgccgcggtgaatacgttcccgggtcttgtacacaccgcccgtcacaccacgagagtttgtaacacccgaagtcggtgaggcaaccttttggagccagccgcctaaggtgggacaaatgattggggtgaagtcgtaacaaggtagccgtatcggaaggtgcggctggatcacctccttt

SEQ ID NO: 84 - DNA, 1480 bp, Microbacterium sp.
  source 1..1480: mol_type = genomic DNA
tacggagagtttgatcctggctcaggatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagctctcatcgcatggtgggggttggaaagattttttggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggaccattccacggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatacaccagaacgggccagaaatggtcaactctttggacactggtgaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaaggagccgtcgaaggtgggatcggtaattaggactaagtcgtaacaaggtagccgtaccggaaggtgcggctggatcacctccttt

SEQ ID NO: 85 - DNA, 992 bp, Methanolacinia petrolearia
  source 1..992: mol_type = genomic DNA
tgcagtcgtacgcttctttttccnccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgnnctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaggaattgacgggggcccgcacagcggtggagcatgnngnttanngancacgcgananntacnnnctnacatcnttgacnctctanagatagagcttcccttcggggcaagtgacng

SEQ ID NO: 86 - DNA, 881 bp, Bacillus velezensis
  source 1..881: mol_type = genomic DNA
cgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggtgttctttgaaaact

SEQ ID NO: 87 - DNA, 1563 bp, Lactobacillus plantarum
  source 1..1563: mol_type = genomic DNA
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctt

SEQ ID NO: 88 - DNA, 1546 bp, Bacillus velezensis
  source 1..1546: mol_type = genomic DNA
tagtgggtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 89 - DNA, 1285 bp, Bacillus subtilis
  source 1..1285: mol_type = genomic DNA
gtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 90 - DNA, 1563 bp, Lactobacillus plantarum; identical to SEQ ID NO: 87
  source 1..1563: mol_type = genomic DNA

SEQ ID NO: 91 - skipped

SEQ ID NO: 92 - DNA, 1265 bp, Bacillus subtilis
  source 1..1265: mol_type = genomic DNA
cgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 93 - DNA, 1548 bp, Leuconostoc mesenteroides
  source 1..1548: mol_type = genomic DNA
attgagagtttgatcctggctcaggatgaacgctggcggcgtgcctaatacatgcaagtcgaacgcacagcgaaaggtgcttgcacctttcaagtgagtggcgaacgggtgagtaacacgtggacaacctgcctcaaggctggggataacatttggaaacagatgctaataccgaataaaacttagtgtcgcatgacaaaaagttaaaaggcgcttcggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtatgggaagaacagctagaataggaaatgattttagtttgacggtaccataccagaaagggacggctaaatacgtgccagcagccgcggtaatacgtatgtcccgagcgttatccggatttattgggcgtaaagcgagcgcagacggtttattaagtctgatgtgaaagcccggagctcaactccggaatggcattggaaactggttaacttgagtgcagtagaggtaagtggaactccatgtgtagcggtggaatgcgtagatatatggaagaacaccagtggcgaaggcggcttactggactgcaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggattgtagtctgcaactcgactacatgaagtcggaatcgctagtaatcgcggatcagcacgccgcggtgaatacgttcccgggtcttgtacacaccgcccgtcacaccatgggagtttgtaatgcccaaagccggtggcctaaccttttaggaaggagccgtctaaggcaggacagatgactggggtgaagtcgtaacaaggtagccgtaggagaacctgcggctggatcacctccttt

SEQ ID NO: 94 - DNA, 1421 bp, Lactobacillus brevis
  source 1..1421: mol_type = genomic DNA
atctgcccagaagcaggggataacacttggaaacaggtgctaataccgtataacaacaaaatccgcatggattttgtttgaaaggtggcttcggctatcacttctggatgatcccgcggcgtattagttagttggtgaggtaaaggcccaccaagacgatgatacgtagccgacctgagagggtaatcggccacattgggactgagacacggcccaaactcctacgggaggcagcagtagggaatcttccacaatggacgaaagtctgatggagcaatgccgcgtgagtgaagaagggtttcggctcgtaaaactctgttgttaaagaagaacacctttgagagtaactgttcaagggttgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggcttaaccggagaagtgcatcggaaactgggagacttgagtgcagaagaggacagtggaactccatgtgtagcggtggaatgcgtagatatatggaagaacaccagtggcgaaggcggctgtctagtctgtaactgacgctgaggctcgaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttctgccaatcttagagataagacgttcccttcggggacagaatgacaggtggtgcatggttgtcgtcagctcgtgtcgtgagatgttgggttaagtcccgcaacgagcgcaacccttattatcagttgccagcattcagttgggcactctggtgagactgccggtgacaaaccggaggaaggtggggatgacgtcaaatcatcatgccccttatgacctgggctacacacgtgctacaatggacggtacaacgagttgcgaagtcgtgaggctaagctaatctcttaaagccgttctcagttcggattgtaggctgcaactcgcctacatgaagttggaatcgctagtaatcgcggatcagcatgccgcggtgaatacgttcccgggccttgtacacaccgcccgtcacaccatgagagtttgtaacacccaaagccggtgagataaccttcgggagtcagccgtctaaggtgggacagatgattagggtgaagtcgtaacaaggtagccgtaggagaacctgcggctggatcacctcctt

SEQ ID NO: 95 - DNA, 1388 bp, Lactobacillus paracasei
  source 1..1388: mol_type = genomic DNA
tgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctccttt

SEQ ID NO: 96 - DNA, 1219 bp, Lactobacillus casei
  source 1..1219: mol_type = genomic DNA
acacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctccttt

SEQ ID NO: 97 - DNA, 1546 bp, Lactococcus garvieae
  source 1..1546: mol_type = genomic DNA
aatgagagtttgatcctggctcaggacgaacgctggcggcgtgcctaatacatgcaagtcgagcgatgattaaagatagcttgctatttttatgaagagcggcgaacgggtgagtaacgcgtgggaaatctgccgagtagcgggggacaacgtttggaaacgaacgctaataccgcataacaatgagaatcgcatgattcttatttaaaagaagcaattgcttcactacttgatgatcccgcgttgtattagctagttggtagtgtaaaggactaccaaggcgatgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaaactctgttgttagagaagaacgttaagtagagtggaaaattacttaagtgacggtatctaaccagaaagggacggctaactacgtgccagcagccgcggtaatacgtaggtcccaagcgttgtccggatttattgggcgtaaagcgagcgcaggtggtttcttaagtctgatgtaaaaggcagtggctcaaccattgtgtgcattggaaactgggagacttgagtgcaggagaggagagtggaattccatgtgtagcggtgaaatgcgtagatatatggaggaacaccggaggcgaaagcggctctctggcctgtaactgacactgaggctcgaaagcgtggggagcaaacaggattagataccctggtagtccacgccgtaaacgatgagtgctagctgtagggagctataagttctctgtagcgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatactcgtgatatccttagagataaggagttccttcgggacacgggatacaggtggtgcatggttgtcgtcagctcgtgtcgtgagatgttgggttaagtcccgcaacgagcgcaacccttattactagttgccatcattaagttgggcactctagtgagactgccggtgataaaccggaggaaggtggggatgacgtcaaatcatcatgccccttatgacctgggctacacacgtgctacaatggatggtacaacgagtcgccaacccgcgagggtgcgctaatctcttaaaaccattctcagttcggattgcaggctgcaactcgcctgcatgaagtcggaatcgctagtaatcgcggatcagcacgccgcggtgaatacgttcccgggccttgtacacaccgcccgtcacaccacggaagttgggagtacccaaagtaggttgcctaaccgcaaggagggcgcttcctaaggtaagaccgatgactggggtgaagtcgtaacaaggtagccgtatcggaaggtgcggctggatcacctccttt

SEQ ID NO: 98 - DNA, 1546 bp, Lactococcus garvieae; identical to SEQ ID NO: 97
  source 1..1546: mol_type = genomic DNA

SEQ ID NO: 99 - DNA, 619 bp, Weissella cibaria
  source 1..619: mol_type = genomic DNA
gatttgaagagcttgctcagatatgacgatggacattgcaaagagtggcgaacgggtgagtaacacgtgggaaacctacctcttagcaggggataacatttggaaacagatgctaataccgtataacaatagcaaccgcatggttgctacttaaaagatggttctgctatcactaagagatggtcccgcggtgcattagttagttggtgaggtaatggctcaccaagacgatgatgcatagccgagttgagagactgatcggccacaatgggactgagacacggcccatactcctacgggaggcagcagtagggaatcttccacaatgggcgaaagcctgatggagcaacgccgcgtgtgtgatgaagggtttcggctcgtaaaacactgttgtaagagaagaatgacattgagagtaactgttcaatgtgtgacggtatcttaccagaaaggaacggctaaatacgtgccagcagccgcggtaatacgtatgttccaagcgttatccggatttattgggcgtaaagcgagcgcagacggttatttaagtctgaagtgaaagccctcagctcaactgaggaattgctttggaaactggatgacttgagtgcagtagagg

SEQ ID NO: 100 - DNA, 1263 bp, Lactobacillus plantarum
  source 1..1263: mol_type = genomic DNA
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatgg

SEQ ID NO: 101 - DNA, 1457 bp, Pediococcus pentosaceus
  source 1..1457: mol_type = genomic DNA
atgagagtttgatcttggctcaggatgaacgctggcggcgtgcctaatacatgcaagtcgaacgaacttccgttaattgattatgacgtacttgtactgattgagattttaacacgaagtgagtggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggcaaaggctcaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgagaccgcgaggttaagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaac

SEQ ID NO: 102 - DNA, 1415 bp, Pichia kudriazevii
  source 1..1415: mol_type = genomic DNA
tccgtaggtgaacctgcggaaggatcattactgtgatttagtactacactgcgtgagcggaacgaaaacaacaacacctaaaatgtggaatatagcatatagtcgacaagagaaatctacgaaaaacaaacaaaactttcaacaacggatctcttggttctcgcatcgatgaagagcgcagcgaaatgcgatacctagtgtgaattgcagccatcgtgaatcatcgagttcttgaacgcacattgcgcccctcggcattccggggggcatgcctgtttgagcgtcgtttccatcttgcgcgtgcgcagagttgggggagcggagcggacgacgtgtaaagagcgtcggagctgcgactcgcctgaaagggagcgaagctggccgagcgaactagactttttttcagggacgcttggcggccgagagcgagtgttgcgagacaacaaaaagctcgacctcaaatcaggtaggaatacccgctgaacttaagcatatcaataagcggaggaaaagaaaccaacagggattgcctcagtagcggcgagtgaagcggcaagagctcagatttgaaatcgtgctttgcggcacgagttgtagattgcaggttggagtctgtgtggaaggcggtgtccaagtcccttggaacagggcgcccaggagggtgagagccccgtgggatgccggcggaagcagtgaggcccttctgacgagtcgagttgtttgggaatgcagctccaagcgggtggtaaattccatctaaggctaaatactggcgagagaccgatagcgaacaagtactgtgaaggaaagatgaaaagcactttgaaaagagagtgaaacagcacgtgaaattgttgaaagggaagggtattgcgcccgacatggggattgcgcaccgctgcctctcgtgggcggcgctctgggctttccctgggccagcatcggttcttgctgcaggagaaggggttctggaacgtggctcttcggagtgttatagccagggccagatgctgcgtgcggggaccgaggactgcggccgtgtaggtcacggatgctggcagaacggcgcaacaccgcccgtcttgaaacatggaccaaggagtctaacgtctatgcgagtgtttgggtgtgaaacccgtacgcgtaatgaaagtgaacgtaggtcggaccccctgccctcggggaggggagcacgatcgaccgatcccgatgtttatcggaaggatttgagtaggagcatagctgttgggacccgaaagatggtgaactatgcctgaatagggtgaagccagaggaaactctggtggaggctcgtagcggttctgacgtgcaaatcgatcgtcgaatttgggtataggggcgaaagactaatcgaaccatctagtagctggttcctgccgaagtttccctcagga

(19) United States
(12) Patent Application Publication
(10) Pub. No.: US 2026/0041722 A1
(43) Pub. Date: Feb. 12, 2026

(54) METHODS AND COMPOSITIONS FOR TREATING MUSCULOSKELETAL DISEASES, TREATING INFLAMMATION, AND MANAGING SYMPTOMS OF MENOPAUSE

(71) Applicant: Solarea Bio, Inc., Waltham, MA (US)

(72) Inventors: Eric Michael Schott, West Roxbury, MA (US); Maria Juliana Soto-Giron, Cambridge, MA (US); Gerardo V. Toledo, Hopkinton, MA (US)

(21) Appl. No.: 19/300,585

(22) Filed: Aug. 14, 2025

Related U.S. Application Data
(63) Continuation of application No. 18/395,925, filed on Dec. 26, 2023, now Pat. No. 12,440,522.
Application No. 18/395,925 is a continuation of application No. 18/181,495, filed on Mar. 9, 2023, now Pat. No. 11,938,158.
Application No. 18/181,495 is a continuation of application No. PCT/US2022/080362, filed on Nov. 22, 2022.
(60) Provisional application No. 63/282,155, filed on Nov. 22, 2021.
(60) Provisional application No. 63/382,666, filed on Nov. 7, 2022.

(51) Int. Cl. A61K 35/747; A61K 9/00; A61K 31/122; A61K 31/355; A61K 31/375; A61K 31/593; A61K 35/744; A61K 36/064; A61K 47/26; A61K 47/46; A61P 15/12

(52) U.S. Cl. CPC A61K 35/747; A61K 9/0056; A61K 31/122; A61K 31/355; A61K 31/375; A61K 31/593; A61K 35/744; A61K 36/064; A61K 47/26; A61K 47/46; A61P 15/12

(57) ABSTRACT

Described herein are methods and compositions for using microbial agents (probiotics) and agents that promote growth of certain microbes (prebiotics) for management (including prevention and treatment) of musculoskeletal disorders, including osteoporosis, osteopenia, Paget's disease, stunting, osteoarthritis, osteomyelitis, and delayed or non-union fractures. Also described herein are methods and compositions for using probiotics and prebiotics for management of inflammation, and symptoms of menopause.

### CROSS-REFERENCE TO RELATED APPLICATIONS

This application is a continuation application of U.S. patent application Ser. No. 18/395,925, filed Dec. 26, 2023, which is a continuation application of U.S. patent application Ser. No. 18/181,495, filed Mar. 9, 2023, now granted as U.S. Pat. No. 11,938,158, which is a continuation of International Application No. PCT/US2022/080362, filed on Nov. 22, 2022, which claims the benefit of and priority to U.S. Provisional Patent Application No. 63/282,155, filed Nov. 22, 2021, and U.S. Provisional Patent Application No. 63/382,666, filed Nov. 7, 2022, the entire disclosures of each of which are incorporated herein by reference in their entirety for all purposes.

### REFERENCE TO A SEQUENCE LISTING

This application contains a Sequence Listing in computer readable form. The computer readable form is incorporated herein by reference. Said XML copy, created on Mar. 9, 2023, is named SBI-013WOC1_SL.xml and is 215,925 bytes in size.

### FIELD OF THE INVENTION

The invention relates generally to methods and compositions for the treatment of musculoskeletal diseases, the treatment of inflammation, and the management of symptoms of perimenopause, menopause, and postmenopause.

### BACKGROUND

Menopause symptoms severely reduce the quality of life of women worldwide. Up to 80% of women may experience menopause symptoms and it is estimated that, in 2030, the at risk groups of peri- and post-menopausal women will reach 1.2 billion globally (Gold et al. (2006) Am J Pub Health 96:1226-1235). The core symptoms are hot flushes (HF) and night sweats (NS), collectively referred to as vasomotor symptoms (VMS); sleep disturbance and other secondary symptoms such as vaginal dryness, urinary urgency, insomnia, irritability, depression, dry skin, dry mouth, dry eyes, headaches, joint and muscle aches, weight gain, racing heart, and changes in libido are also often present (Lim et al. (2020) J Clin Med 9:2173). These symptoms are largely a consequence of natural endogenous estrogen decline and dysregulation during peri-and post-menopause (Lambert et al. (2017) PLOS One 12 (6): e0176590). Hormone therapy (HT) is the current gold standard treatment for VMS. However, substantial evidence supports that therapy increases cancer risk in estrogen receptor (ER) a rich tissues (Lambert et al. (2017) PLOS One 12 (6): e0176590). Thus, there is a significant need for new approaches to alleviating VMS in peri- and post-menopausal women.

Intriguingly, menopause has been demonstrated to shift the composition of the gut microbiome (Choi et al. (2017) J Microbiol Biotechnol 27:2228-2236; Santos-Marcos et al. (2018) Maturitas 116:43-53) and increase gastrointestinal permeability in both animal and clinical studies (Li et al. (2016) J Clin Investig 126:2049-2063). Furthermore, the microbiome has been shown to effect circulating levels of estrogen, and thus may be a therapeutic target to improve menopausal symptoms (Flores et al. (2012) J Transl Med 10:253).

Probiotics have emerged as an intriguing new approach to treating VMS. Lactobacillus gasseri was shown to reduce postmenopausal symptoms in ovariectomized rats (Lee et al. (2021) J Microbiol Biotechnol 31 (9): 1-10). Further, recent clinical trials with probiotics have shown promise, leading to a reduction in the VMS associated with menopause (Lambert et al. (2017) PLOS One 12 (6): e0176590, Lim et al. (2020) J Clin Med 9:2173). Despite the efficacy observed in these studies, the mechanism of how probiotics impact VMS is not completely understood. One potential mechanism is via alteration of dietary isoflavone metabolite availability by probiotics, increasing the uptake of these estrogen receptor agonists (Lambert et al. (2017) PLOS One 12 (6): e0176590). Therefore, there is a need for novel treatments for alleviating menopausal symptoms that utilize the beneficial properties of probiotics.

### SUMMARY OF THE INVENTION

The disclosure relates generally to methods and compositions for the treatment of musculoskeletal diseases, the treatment of inflammation, and the management of symptoms of perimenopause, menopause, and postmenopause.

For example, in one aspect, provided herein is a dietary supplement comprising a combination of four heterologous microbes consisting of Lactobacillus brevis, Lactobacillus plantarum, Leuconostoc mesenteroides, and Pichia kudriavzevii formulated in an amount effective for lessening a decrease in, maintaining, or improving bone health in a subject, wherein the dietary supplement is formulated for oral delivery. In certain embodiments, lessening a decrease in, maintaining, or improving bone health in the subject comprises lessening a decrease in, maintaining, or improving bone mineral density (BMD) in the subject. In certain embodiments, lessening a decrease in, maintaining, or improving bone health in the subject comprises lessening a decrease in, maintaining, or improving trabecular bone score (TBS) in the subject.

In certain embodiments, the heterologous microbes are co-formulated as a synthetic microbial consortium in a unit dose.

In certain embodiments, the dietary supplement is formulated as a medical food or a pharmaceutical composition.

In certain embodiments, the unit dosage amount is a dosage amount of about 1.0×10⁸ to 1.0×10¹² CFU of each of the heterologous microbes. In certain embodiments, the unit dosage amount is a dosage amount of about 2.5×10⁹ to 3.0×10¹⁰ CFU of each of the heterologous microbes.

In certain embodiments, following administration of the dietary supplement to the subject over a period of time: (i) BMD in the subject is maintained or improved as compared to a suitable control, and/or (ii) a decrease in BMD in the subject is less severe as compared to a suitable control. In certain embodiments, the suitable control comprises (i) a control group that has not been administered the dietary supplement, (ii) the subject's BMD prior to the first administration of the dietary supplement to the subject and/or (iii) the rate of decline of the subject's BMD prior to the first administration of the dietary supplement to the subject.

In certain embodiments, BMD is measured as areal BMD (aBMD) or volumetric BMD (vBMD).

In certain embodiments, following administration of the dietary supplement to the subject over a period of time: (i) the subject's TBS is maintained or improved as compared to a suitable control, and/or (ii) a decrease in TBS in the subject is less severe as compared to a suitable control. In certain embodiments, the suitable control comprises (i) a control group that has not been administered the dietary supplement, (ii) the subject's TBS prior to the first administration of the dietary supplement to the subject and/or (iii) the rate of decline of the subject's TBS prior to the first administration of the dietary supplement to the subject.

In certain embodiments, the taxonomic or functional composition of the microbiome of the subject is altered after administration of the dietary supplement to the subject, as compared to a suitable control. In certain embodiments, the suitable control comprises (i) a control group that has not been administered the dietary supplement, and/or (ii) the taxonomic or functional composition of the microbiome of the subject prior to the first administration of the dietary supplement.

In certain embodiments, the microbiome is altered by an increase in the abundance of the microbial species present in the dietary supplement. In certain embodiments, the microbiome is altered by increased gene abundance of vitamin K2 biosynthesis pathways.

In certain embodiments, administration of the dietary supplement to the subject results in (i) altering the amount of at least one biochemical marker of bone turnover in the subject and/or (ii) altering the amount of at least one circulatory inflammatory cytokine or marker of inflammation in the subject, wherein the amount of the at least one biochemical marker of bone turnover and/or at least one circulatory inflammatory cytokine or marker of inflammation is altered as compared to a suitable control. In certain embodiments, the suitable control comprises (a) a control group that has not been administered the dietary supplement, and/or (b) the amount of the at least one biochemical marker of bone turnover and/or the at least one circulatory inflammatory cytokine or marker of inflammation in the subject prior to the first administration of the dietary supplement.

In certain embodiments, the at least one biochemical marker of bone turnover comprises CTX and/or PINP. In certain embodiments, the amount of CTX decreases, the amount of PINP increases, and/or the ratio of PINP to CTX increases.

In certain embodiments, the at least one circulatory inflammatory cytokine or marker of inflammation is selected from the group consisting of CRP, IL-17, TNF, IL-1B, IL-4, RANKL, and IFNγ. In certain embodiments, the amount of the at least one circulatory inflammatory cytokine or marker of inflammation decreases.

In another aspect, provided herein is a a dietary supplement comprising a combination of four heterologous microbes consisting of Lactobacillus brevis, Lactobacillus plantarum, Leuconostoc mesenteroides, and Pichia kudriavzevii for improving one or more symptoms of menopause in a subject, wherein the dietary supplement is formulated for oral delivery.

In certain embodiments, following administration of the dietary supplement to the subject over a period of time, the one or more symptoms of menopause are improved as compared to a suitable control. In certain embodiments, the suitable control is (i) a control group that has not been administered the dietary supplement and/or (ii) the presence or severity of the subject's one or more symptoms prior to the first administration of the dietary supplement.

In certain embodiments, the one or more symptoms of menopause are selected from the group consisting of: hot flushes, sweating, episodes of sweating, night sweats, heart discomfort, unusual awareness of heart beat, heart skipping, heart racing, heart tightness, depressive mood, feeling down, feeling sad, feeling on verge of tears, lack of drive, mood swings, irritability, feeling nervous, inner tension, feeling aggressive, anxiety, inner restlessness, feeling panicky, physical exhaustion, mental exhaustion, general decrease in performance, impaired memory, decrease in concentration, forgetfulness, sexual problems, change in sexual desire, change in sexual activity, change in sexual satisfaction, bladder problems, difficulty in urinating, increased need to urinate, bladder incontinence, dryness of the vagina, sensation of dryness or burning in the vagina, difficulty with sexual intercourse, joint and muscular discomfort, pain in the joints, and rheumatoid arthritis. In certain embodiments, the one or more symptoms of menopause comprise a vasomotor symptom, wherein the vasomotor symptom is selected from hot flushes, sweating, night sweats, and combinations thereof.

In certain embodiments, severity of the one or more symptoms of menopause is measured by the Menopause Rating Scale (MRS), optionally wherein the improvement of the symptom is measured in the same subject about 2 months, 4 months, 6 months, 8 months, 10 months, and/or 12 months after the first administration of the dietary supplement.

In certain embodiments, the dietary supplement further comprises a prebiotic. In certain embodiments, the prebiotic is oligofructose and/or a dried fruit or vegetable powder. In certain embodiments, the dried fruit or vegetable powder is a dried berry powder. In certain embodiments, the prebiotic is dried blueberry powder.

In certain embodiments, the dietary supplement further comprises a bulking agent. In certain embodiments, the bulking agent is magnesium stearate.

In certain embodiments of any of the foregoing dietary supplements, at least one of the heterologous microbes comprises a 16S rRNA or fungal ITS sequence, having at least 97%, at least 98%, at least 98.5%, at least 99%, or at least 100% similarity to any one of SEQ ID NOs: 93, 94, 100 and 102 at the 16S rRNA or fungal ITS sequence.

In certain embodiments, the dietary supplement further comprises at least one additional microbe from Table 1 or Table 2.

In certain embodiments of a dietary supplement disclosed herein, the unit dose further comprises a cryoprotectant present in an effective amount to extend survival of the heterologous microbes after thawing the unit dose from a cryogenic temperature.

In another aspect, provided herein is a method of producing a dietary supplement comprising a combination of four heterologous microbes consisting of Lactobacillus brevis, Lactobacillus plantarum, Leuconostoc mesenteroides, and Pichia kudriavzevii for lessening a decrease in, maintaining, or improving bone health in a subject, the method comprising co-formulating the heterologous microbes as a synthetic microbial consortia in a unit dose formulated for oral administration to the subject.

In another aspect, provided herein is a method of (i) lessening a decrease in, maintaining, or improving bone health in a subject and/or (ii) improving one or more symptoms of menopause in a subject, the method comprising administering to the subject a therapeutically effective amount of each of four heterologous microbes consisting of Lactobacillus brevis, Lactobacillus plantarum, Leuconostoc mesenteroides, and Pichia kudriavzevii. In certain embodiments, lessening a decrease in, maintaining, or improving bone health in the subject comprises (i) lessening a decrease in, maintaining, or improving bone mineral density (BMD) in the subject and/or (ii) lessening a decrease in, maintaining, or improving trabecular bone score (TBS) in the subject.

In certain embodiments, the subject has, is diagnosed with, or is at risk for one or more of the group consisting of: osteoporosis, osteopenia, osteoarthritis, suboptimal fracture healing, osteomyelitis, Paget's disease, stunting, and delayed or non-union fractures.

### BRIEF DESCRIPTION OF FIGURES

These and other features, aspects, and advantages of the present invention will become better understood with regard to the following description, and accompanying drawings, where:

FIG. 1 summarizes the Defined Microbial Assemblage manufacturing process described in Example 1.

FIG. 2 are graphs summarizing acetate production by four microbial strains, either individually or in combination. DMA-04 constituent strains (SBS4254, SBS4263, SBS4255, and SBS4260) were used to inoculate medium containing blueberry powder and oligofructose, and supernatant acetate levels were quantified by gas chromatography after a 24-hour incubation period. Strains were tested either individually or in a combination (“DMA-04”). Acetate levels were quantified as a percentage of the combination treatment. The sum of the mean acetate levels produced by individual strains is illustrated as a striped bar (“Sum of DMA-04 Strains”). Data represent mean+SD; n=2.

FIG. 3 are graphs summarizing acetate production by mixed cultures inoculated with four strains of microbes. Each culture was inoculated with three of the four constituent strains of DMA-04; the remaining DMA-04 strain was substituted with a different strain of the same species (either L. brevis SBI4879, P. kudriavzevii SBI4870, L. mesenteroides SBI4914, or L. plantarum SBI4911). A mixed culture containing all four constituent strains of DMA-04 was used as a control. Strains were used to inoculate medium containing blueberry powder and oligofructose, and supernatant acetate levels were quantified by gas chromatography after a 24-hour incubation period. Acetate levels of the “substitute” cultures were quantified as a percentage of the DMA-04 4-strain control culture. Data represent mean+SD; n=2.

FIG. 4 is a diagram summarizing the experimental protocol described in Example 4, which was used to analyze the protective effect of a Defined Microbial Assemblage against ovariectomy-induced bone loss in mice.

FIG. 5A-5D is a graph depicting the ability of DMA-04 or Lactobacillus paracasei to protect against an ovariectomy-(OVX-) induced decrease in bone mineral density (BMD) in a murine estrogen withdrawal model of osteoporosis. OVX mice were treated with L. paracasei or DMA-04 by oral gavage. Sham-treated (non-OVX) mice and OVX-mice treated with water and were used as controls. BMD was measured 42 days post-OVX and was quantified relative to the BMD of sham-treated mice. Data represent mean+/−SEM. n=12 per treatment. Significant differences between groups were identified by one-way ANOVA with Tukey multiple comparison tests (* P<0.05). FIG. 5A summarizes the results for total-body BMD, as measured by Faxitron DXA scan. FIG. 5B summarizes the results for total-body BMD, as measured by Pixi DXA scan. FIG. 5C summarizes the results for spine BMD, as measured by Pixi DXA scan. FIG. 5D summarizes the results for femur BMD, as measured by Pixi DXA scan.

FIG. 6 summarizes detection of DMA-04 strains in the gut microbiome of participants prior to treatment with DMA-04 or a placebo (“Day 0”), after 28 days of twice-daily administration of DMA-04 or a placebo (“Day 28”), and after a 28-day washout period of non-administration of DMA-04 or the placebo (“Day 56”). Participant stool samples were obtained at the indicated timepoints, and the relative abundance of the indicated strain was determined by metagenomic sequencing. Statistical significance of differences in relative abundance of the bacterial strains between DMA-04 and placebo treatments was evaluated at each time point using an LDA Effect Size (LefSe) analysis, and an LDA score of ≥2.0 was considered significant (*P <0.05). Mann-Whitney test was used to compare the ITS coverage of P. kudriavzevii between DMA-04 and placebo treatments at each time point (** P<0.01). Boxes represent medians and upper and lower quartiles. Whiskers represent minimum and maximum values.

FIG. 7A-7B summarize differences in microbial taxa and metabolic pathways in subject gut microbiomes before (FIG. 7A) and after (FIG. 7B) 28 days of twice-daily administration of DMA-04 or a placebo. Participant stool samples were collected at the indicated timepoints and analyzed via metagenomic sequencing. Microbial taxa and metabolic pathways with differential abundance between placebo and DMA-04 treatment groups were determined by LDA Effect Size (LefSe) analysis (p<0.05 and LDA score ≥2.0). The LDA score corresponds to the degree of difference in the magnitude of relative abundances of taxa and pathways that differ between the two groups.

FIG. 8 summarizes differences in metabolic pathways associated with vitamin K biosynthesis in subject gut microbiomes after 28 days of twice-daily administration of DMA-04 or a placebo. Participant stool samples were collected on Day 28 and analyzed via metagenomic sequencing. Metabolic pathways with differential abundance between treatment groups at Day 28 were determined by LefSe analysis (p<0.05 and LDA score ≥2.0). PWY-5845: superpathway of menaquinol-9 biosynthesis; PWY-5850: superpathway of menaquinol-6 biosynthesis; PWY-5860: superpathway of demethylmenaquinol-6 biosynthesis I; PWY-5862: superpathway of demethylmenaquinol-9 biosynthesis; PWY-5896: superpathway of menaquinol-10 biosynthesis. Boxes represent medians and upper and lower quartiles. Whiskers represent minimum and maximum values.

FIG. 9 is a diagram summarizing the clinical study protocol of DMA-04 described in Example 6.

### DETAILED DESCRIPTION

### Advantages and Utility

Briefly, and as described in more detail below, described herein are methods and compositions for using microbial agents (probiotics) and agents that promote growth of certain microbes (prebiotics) for management (including prevention and treatment) of musculoskeletal disorders, including osteoporosis and osteopenia, for the management of inflammation, and for the management of symptoms of menopause, perimenopause and postmenopause.

Several features of the current approach should be noted. It is based on development of synergistic combinations of microbes as on those found in fruits and vegetables consumed as part of a plant-based diet. The combinations are based, in part, on analyses of biochemical pathways catalyzed by genes in these microbes and selection of microbial combinations that promote beneficial metabolic changes in a subject through the biochemical reactions they catalyze such as, but not limited to, the production of short chain fatty acids (SCFA).

Advantages of this approach are numerous. They include reduction of the morbidity associated with musculoskeletal disorders, such as osteoporosis or osteopenia, without the use of traditional drugs and the side effects they can sometimes cause. This approach may also be used to reduce inflammation and alleviate symptoms or effects of menopause, perimenopause or postmenopause.

In certain aspects, this disclosure is useful for providing health benefits associated with consumption of a plant-based diet, as the diet microbes and fibers are delivered in concentrated form. This can reduce the burden on a subject to ingest potentially unreasonable or inconvenient amounts of particular plants and/or plant-based products, such as fermented foods.

### Definitions

Terms used in the claims and specification are defined as set forth below unless otherwise specified.

The term “ameliorating” refers to any therapeutically beneficial result in the treatment of a disease state, e.g., a musculoskeletal disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

As used herein, the term “derived from” includes microbes immediately taken from an environmental sample and also microbes isolated from an environmental source and subsequently grown in pure culture. The term “derived from” also includes material isolated from the recited source, and materials obtained using the isolated materials (e.g., cultures of microorganisms made from microorganisms isolated from the recited source).

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

In some cases, alignment of an entire sequence is not necessary for identification or comparison purposes regarding a microbial entity. In such a case, a so-called diagnostic subsequence can be used. The term “diagnostic subsequence” refers to a portion of a known sequence which would be identified and used by one of skill in the art to identify or compare two or more microbial entities. One, non-limiting example is utilization of subsequences of 16S rRNA sequences found in Asgari et al (2018, bioRxiv).

The term “effective amount” is an amount that is effective to ameliorate or manage a symptom of a disease, disorder, state, or condition. An effective amount can also be an amount effective for prophylaxis of a particular disease, disorder, state, or condition (e.g., symptoms of menopause). More generally, an effective amount is an amount sufficient to produce a desired effect, e.g., an amount effective for alteration of the microbial content of a subject's microbiota. As used herein, nonlimiting examples of a disease, disorder, state, or condition include, e.g., osteoporosis, osteopenia, chronic inflammation, menopause, perimenopause, and postmenopause.

The term “dietary supplement”, as used herein refers to a substance that is not a conventional food and that is manufactured to be administered to a subject over a period of time, wherein the substance is an addition to the subject's diet and is effective to produce a desired effect when administered to the subject over a period of time. In certain embodiments, the desired effect is treating, ameliorating, preventing, or managing one or more symptoms of a disease, disorder, state, or condition in the subject.

The term “medical food”, as used herein refers to a dietary supplement which is formulated to be consumed or administered enterally with or without the supervision of a physician and which is intended for the dietary management of a disease, state, disorder, or condition or one or more symptoms thereof.

The term “menopause”, as used herein in relation to a subject, refers to the time at which 12 months have elapsed since the last menstruation of the subject. The term “perimenopause”, as used herein in relation to a subject, refers to the period of time about 6 months to about 10 years prior to menopause in the subject, and ending at menopause. In some embodiments, perimenopause is characterized by a decrease in estrogen levels/production, irregular menstrual cycles, and/or an alteration of menstrual cycle patterns in the subject. The term “postmenopause”, as used herein in relation to a subject, refers to the period of time beginning 12 months after the last menstruation of the subject and concluding at the end of the subject's life

The term “symptoms of menopause”, as used herein includes symptoms of menopause, perimenopause, and postmenopause, and includes, but is not limited to, hot flushes, sweating and night sweats, collectively referred to as vasomotor symptoms; sleep disturbance and other secondary symptoms such as vaginal dryness, urinary urgency, insomnia, irritability, depression, dry skin, dry mouth, dry eyes, headaches, joint and muscle aches, weight gain, racing heart, changes in libido, and increased risk or occurrence of cancer.

The term “defined microbial assemblage” or “DMA” refers to a combination of two or more microbial strains (e.g., bacterial or fungal) wherein the two or more microbial strains are chosen because they are predicted to achieve a particular synergistic result when applied in concert. DMA compositions may further comprise prebiotics or other fiber sources predicted to increase the desired effect of the microbial strains applied. A DMA is rationally designed to achieve a particular benefit, such as, but not limited to, increased SCFA production in the gut lumen.

As used herein, the term “treating” includes abrogating, substantially inhibiting, slowing or reversing the progression of a disease, disorder, state, or condition, or clinical or aesthetical symptoms of a disease, disorder, state, or condition.

As used herein, the term “preventing” includes completely or substantially reducing the likelihood or occurrence or the severity of initial clinical or aesthetical symptoms of a disease, disorder, state, or condition.

As used herein, the term “about” includes variation of up to approximately +/−10% and that allows for functional equivalence in the product, composition, or method unless otherwise indicated or inferred. Where the use of the term “about” appears before a quantitative value, the present invention also includes the specific quantitative value itself, unless specifically stated otherwise.

As used herein, the term “colony-forming unit” or “cfu” is an individual cell that is able to clone itself into an entire colony of identical cells.

As used herein all percentages are weight percent unless otherwise indicated.

As used herein, “viable organisms” are organisms that are capable of growth and proliferation. In some embodiments, viability can be assessed by numbers of colony-forming units that can be cultured. In some embodiments viability can be assessed by other means, such as, but not limited to, quantitative polymerase chain reaction.

“Microbiota” refers to the community of microorganisms that occur (sustainably or transiently) in and on a plant or an animal subject, typically a mammal such as a human, including eukaryotes, archaea, bacteria, and viruses (including bacterial viruses i.e., phage).

“Microbiome” refers to the genetic content of the communities of microbes that live inside and on the human body, or inside or outside a plant, both sustainably and transiently, including eukaryotes, archaea, bacteria, and viruses (including bacterial viruses (i.e., phage)), wherein “genetic content” includes genomic DNA, RNA such as ribosomal RNA, the epigenome, plasmids, and all other types of genetic information.

As used herein, the term “prebiotic” refers to a substance or composition (either alone or in combination with one or more other substances) that enhances or supports the growth of microbes. In some embodiments, a prebiotic enhances or supports the growth of a probiotic in a subject. In some embodiments, a prebiotic enhances or supports the growth of a DMA in a subject.

The term “subject” refers to any organism to be treated by the methods and compositions described herein. Such organisms preferably include, but are not limited to, mammals (e.g. murines, simians, equines, bovines, porcines, canines, felines, and the like), and more preferably include humans. In some embodiments, the subject may be suffering from a dysbiosis, including, but not limited to, an infection due to a gastrointestinal pathogen or may be at risk of developing or transmitting to others an infection due to a gastrointestinal pathogen. In some embodiments, the subject may have or be at risk for a musculoskeletal disease such as osteoporosis or osteopenia. In some embodiments, the subject may be in menopause, perimenopause, postmenopause, or may be undergoing or may have undergone a menopause transition. In some embodiments, the subject may have chronic inflammation.

The “colonization” of a host organism includes the non-transitory residence of a bacterium or other microscopic organism. In some embodiments, a “colonizing” microbial strain may remain in in a host subject's gastrointestinal tract for a period of time following administration of a composition comprising said microbial strain to the subject. For example, in some embodiments, a “colonizing” microbe or strain may remain in the host subject's gastrointestinal tract for at least 1 hour, at least 6 hours, at least 12 hours, at least 1 day, at least 7 days, at least one month, at least one year, etc., following administration of a composition comprising said microbial strain to the subject.

As used herein, “reducing colonization” of a host subject's gastrointestinal tract (or any other microbiotal niche) by a pathogenic bacterium includes a reduction in the residence time of the pathogen in the gastrointestinal tract as well as a reduction in the number (or concentration) of the pathogen in the gastrointestinal tract or adhered to the luminal surface of the gastrointestinal tract. Measuring reductions of adherent pathogens may be demonstrated, e.g., by a biopsy sample, or reductions may be measured indirectly, e.g., by measuring the pathogenic burden in the stool of a mammalian host.

A “combination” of two or more bacteria includes the physical co-existence of the two bacteria, either in the same material or product or in physically connected products, as well as the temporal co-administration or co-localization of the two bacteria.

As used herein “heterologous” designates organisms to be administered that are not naturally present in the same proportions as in the therapeutic composition as in subjects to be treated with the therapeutic composition. These can be organisms that are not normally present in individuals in need of the composition described herein, or organisms that are not present in sufficient proportion in said individuals. These organisms can comprise a synthetic composition of organisms derived from separate plant sources or can comprise a composition of organisms derived from the same plant source, or a combination thereof.

As used herein, the terms “administer”, “administering”, or “administration” refer to the placement or delivery of a composition or substance (e.g., a DMA composition described herein) onto or into a subject, e.g. orally, rectally, parenterally, or intranasally. In some embodiments, one or more persons who are not the subject (e.g., an investigator or medical worker) may administer a composition or substance to the subject. In some embodiments, the subject may administer a composition or substance to themselves.

In some embodiments, compositions and methods disclosed herein can be used to treat osteoporosis or osteopenia. Osteoporosis is a systemic skeletal disease characterized by decreasing bone mass and microarchitectural deterioration of bone tissue that leads to an increased risk for bone fragility and fracture. In patients without fragility fracture, osteoporosis is often diagnosed by low bone mineral density (BMD). The international reference standard for the description of osteoporosis in postmenopausal women and in men is a femoral neck or lumbar spine BMD of 2.5 standard deviations (SD) or more below the young female adult mean. Osteopenia is a less severe form of low BMD, defined by the international standard as between 1 and 2.5 SD below the young female average. In certain embodiments, “osteoporosis or osteopenia” indicates a condition where the subject's bone mass per unit volume is reduced (e.g., as compared to an appropriate population average). Osteoporosis indicates bone mass reduction to a level below that required for the adequate mechanical support function of the bone. Osteopenia is a milder disease where bone mass per unit is reduced but not to the extent seen in osteoporosis. Patients with osteopenia may subsequently suffer from osteoporosis. Trabecular bone score (TBS) can also be used as a risk marker and/or diagnostic marker for osteoporosis or osteopenia.

As used herein, the term “bone health” refers to an assessment of bone quality, bone integrity, bone density, and/or bone turnover. Bone health may be assessed using any appropriate standard, metric, or method known in the art, including, e.g., by bone mineral density (BMD) or trabecular bone score (TBS).

As used herein, “bone density” indicates “bone mineral density” (BMD). In some embodiments, compositions and methods disclosed herein can be used to improve or increase BMD.

In some embodiments, compositions and methods disclosed herein can be used to treat osteoarthritis. As used herein, the term “osteoarthritis” (abbreviated as “OA”), refers to the disease also known as osteoarthrosis and degenerative joint disease, characterized by inflammation and damage to, or loss of cartilage in any joint or joints, and joint pain. Clinical standards for diagnosing osteoarthritis in subjects including mammalian subjects such as canines and humans are well known and include, for example, swelling or enlargement of joints, joint tenderness or pain, decreased range of motion in joints, visible joint deformities such as bony growths, and crepitus. Symptoms can be identified by clinical observation and history, or imaging including MRI and X-ray. Criteria for diagnosing the presence or absence of OA and severity or degree of OA include but are not limited to the ACR Criteria for knee OA (R. Altman et al., Development of criteria for the classification and reporting of osteoarthritis: Classification of osteoarthritis of the knee: Diagnostic and Therapeutic Criteria Committee of the American Rheumatism Association. Arthritis Rheum. August 29 (8): 1039-1049 (1986)), functional status criteria according to WOMAC (N. Bellamy et al., 1988, Validation study of WOMAC: a health status instrument for measuring clinically important patient relevant outcomes to antirheumatic drug therapy in patients with osteoarthritis of the hip or knee. J Rheumatol 15:1833-1840), and radiological standards for evaluating OA disease severity according to the Kellgren and Lawrence method for knee OA (Kellgren, J. H. and J. S. Lawrence, Radiological assessment of osteo-arthrosis. Ann Rheum Dis 16:494-502).

In some embodiments, compositions and methods disclosed herein can be used to improve fracture healing. The term “fracture”, as used herein, refers to a disruption in the integrity of a living bone involving injury to bone marrow, periosteum, and adjacent soft tissues. Many types of fractures exist such as, for example, pathological, stress, non-union, delayed-union, and greenstick fractures. A fracture includes open and closed fractures.

The term “fracture line” refers to the line across where disruption of the integrity of the living bone has occurred.

The term “non-union” fracture refers to the fractures which are not completely healed nine months after the initial fracture. These are commonly found in clavicle fractures that are not healed usually within three months, and are usually painful and require surgical fixation.

The term “delayed-union” refers to a fracture that has not healed at least about six months post injury.

In some embodiments, compositions and methods disclosed herein can be used to prevent or treat osteomyelitis. As used herein, “osteomyelitis” is defined as inflammation of the bone or bone marrow. In some embodiments, osteomyelitis is caused by an infection.

In some embodiments, compositions and methods disclosed herein can be used to improve trabecular bone score (TBS).

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be comprised in and/or selected from a list of recited elements or components, it should be understood that the element or component can be any one of the recited elements or components, or the element or component can be selected from a group consisting of two or more of the recited elements or components.

Throughout this application, various embodiments of this disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable subcombination or as suitable in any other described embodiment of the disclosure. Certain features described in the context of various embodiments are not to be considered essential features of those embodiments, unless the embodiment is inoperative without those elements.

It must be noted that, as used in the specification and the appended claims, the singular forms “a,” “an” and “the” include plural referents unless the context clearly dictates otherwise.

### Compositions

In certain embodiments, compositions of the disclosure comprise probiotic compositions formulated for administration and/or consumption, with a prebiotic and any necessary or useful excipient. In other embodiments, compositions of the disclosure comprise probiotic compositions formulated for consumption without a prebiotic. Probiotic compositions are preferably isolated from foods normally consumed raw and isolated for cultivation. In some embodiments, microbes are isolated from different foods normally consumed raw, but multiple microbes from the same food source may be used.

It is known to those of skill in the art how to identify microbial strains. Bacterial strains are commonly identified by 16S rRNA gene sequence. Fungal species can be identified by sequence of the internal transcribed space (ITS) regions of rDNA.

One of skill in the art will recognize that the 16S rRNA gene and the ITS region comprise a small portion of the overall genome, and so sequence of the entire genome (whole genome sequence) may also be obtained and compared to known species.

Additionally, multi-locus sequence typing (MLST) is known to those of skill in the art. This method uses the sequences of 7 known bacterial genes, typically 7 housekeeping genes, to identify bacterial species based upon sequence identity of known species as recorded in the publicly available PubMLST database. Housekeeping genes are genes involved in basic cellular functions.

In certain embodiments, bacterial entities are identified by comparison of the 16S rRNA sequence to those of known bacterial species, as is well understood by those of skill in the art. In certain embodiments, fungal species are identified based upon comparison of the ITS sequence to those of known species (Schoch et al PNAS 2012). In certain embodiments, microbial strains are identified by whole genome sequencing and subsequent comparison of the whole genome sequence to a database of known microbial genome sequences. While microbes identified by whole genome sequence comparison, in some embodiments, are described and discussed in terms of their closest defined genetic match, in certain embodiments as indicated by 16S rRNA gene sequence, it should be understood that these microbes are not identical to their closest genetic match and are novel microbial entities. This can be shown by examining the Average Nucleotide Identity (ANI) of microbial entities of interest as compared to the reference strain that most closely matches the genome of the microbial entity of interest (see, e.g., WO2020051379A1).

In other embodiments, microbial entities described herein are functionally equivalent to previously described strains with homology at the 16S rRNA or ITS region. In certain embodiments, functionally equivalent bacterial strains have at least 95% identity at the 16S rRNA region and functionally equivalent fungal strains have at least 95% identity at the ITS region. In certain embodiments, functionally equivalent bacterial strains have at least 96% identity at the 16S rRNA region and functionally equivalent fungal strains have at least 96% identity at the ITS region. In certain embodiments, functionally equivalent bacterial strains have at least 97% identity at the 16S rRNA region and functionally equivalent fungal strains have at least 97% identity at the ITS region. In certain embodiments, functionally equivalent bacterial strains have at least 98% identity at the 16S rRNA region and functionally equivalent fungal strains have at least 98% identity at the ITS region. In certain embodiments, functionally equivalent bacterial strains have at least 99% identity at the 16S rRNA region and functionally equivalent fungal strains have at least 99% identity at the ITS region. In certain embodiments, functionally equivalent bacterial strains have at least 99.5% identity at the 16S rRNA region and functionally equivalent fungal strains have at least 99.5% identity at the ITS region. In certain embodiments, functionally equivalent bacterial strains have 100% identity at the 16S rRNA region and functionally equivalent fungal strains have 100% identity at the ITS region.

16S rRNA sequences for strains tolerant of relevant stressors are found in SEQ ID NOs 1-102 (Table 2). 16S rRNA is one way to classify bacteria into operational taxonomic units (OTUs). Bacterial strains with 97% sequence identity at the 16S rRNA locus are considered to belong to the same OTU. A similar calculation can be done with fungi using the ITS locus in place of the bacterial 16S IRNA sequence.

In some embodiments, the invention provides a probiotic composition for the treatment of, e.g., osteoporosis, osteopenia, Paget's disease, stunting, inflammation, menopause symptoms, perimenopause symptoms, or postmenopause symptoms. In some embodiments, the composition comprises a mixture of lactic acid bacteria, such as Pediococcus spp, Leuconostoc spp,

Leuconostoc mesenteroides, Lactobacillus spp, Lactobacillus crispatus, Lactobacillus plantarum, Lactobacillus brevis, and/or Lactobacillus reuteri, optionally combined with non-lactic acid bacteria such as one or more of the non-lactic acid bacteria described in Table 1 or

Table 2. In some embodiments, the invention provides a fermented probiotic composition for the treatment of bone diseases, inflammation, or symptoms of menopause, perimenopause, or postmenopause, comprising a mixture of Lactobacillus plantarum, Lactobacillus brevis, and/or Leuconostoc mesenteroides and at least one non-lactic acid bacterium, preferably a bacterium classified as a gamma proteobacterium or a filamentous fungus or yeast (e.g. Pichia kudrivzevii). Some embodiments comprise the probiotic being in a capsule or microcapsule adapted for enteric delivery.

In some embodiments, the invention provides a probiotic composition comprising a fungal microbe. In some embodiments, the fungal microbe is a yeast, such as Pichia kudriavzevii, Candida krusei, Issatchenkia orientalis, and/or Candida glycerinogenes. In some embodiments, the fungal microbe is selected from a fungal microbe listed in Table 1 or Table 2.

In some embodiments, the invention provides a probiotic composition comprising a mixture of lactic acid bacteria (such as Pediococcus spp, Leuconostoc spp, Leuconostoc mesenteroides, Lactobacillus spp, Lactobacillus crispatus, Lactobacillus plantarum, Lactobacillus brevis, Lactobacillus reuteri) and fungal microbes (such as yeast, e.g. Pichia kudriavzevii, Candida krusei, Issatchenkia orientalis, and/or (′andida glycerinogenes). In some embodiments, the composition comprises at least one, at least two, at least three, at least four, at least five, etc., heterologous lactic acid bacteria and at least one, at least two, at least three, at least four, at least five, etc. heterologous fungal microbes. In some embodiments, the composition comprises three species of lactic acid bacteria. In some embodiments, the composition comprises Lactobacillus brevis, Lactobacillus plantarum, and Leuconostoc mesenteroides. In some embodiments, the composition comprises one species of fungal microbe. In some embodiments, the composition comprises Pichia kudriavzevii. In some embodiments, the composition comprises Lactobacillus brevis, Lactobacillus plantarum, Leuconostoc mesenteroides, and Pichia kudriavzevii.

In some embodiments, the compositions disclosed herein are derived from edible plants and can comprise a mixture of microorganisms, comprising bacteria, fungi, archaea, and/or other indigenous or exogenous microorganisms, all of which work together to form a microbial ecosystem with a role for one or more of its members. In some embodiments, the compositions disclosed herein comprise a microorganism listed in Table 1, which were isolated from edible plant samples. The isolation and identification of the strains in Table 1 is described in WO2020051379A1, which is hereby incorporated by reference in its entirety.

TABLE 1
Bacteria identified in a 15-sample survey of edible plants and
identified by whole genome matching to reference genomes.

| Strain identified by k-mer | Strain | |
|---|---|---|
| based on entire genome | number | Collection |
| Acinetobacter baumannii | — | |
| Acinetobacter soli | — | |
| Acinetobacter 41764 Branch | — | |
| Acinetobacter 41930 Branch | — | |
| Acinetobacter 41981 Branch | — | |
| Acinetobacter 41982 Branch | — | |
| Acinetobacter baumannii 348935 | — | |
| Acinetobacter baumannii 40298 Branch | — | |
| Acinetobacter beijerinckii 41969 Branch | — | |
| Acinetobacter beijerinckii CIP 110307 | CIP 110307 | WFCC |
| Acinetobacter bohemicus ANC 3994 | — | |
| Acinetobacter guillouiae 41985 Branch | — | |
| Acinetobacter guillouiae 41986 Branch | — | |
| Acinetobacter gyllenbergii 41690 Branch | — | |
| Acinetobacter haemolyticus TG19602 | — | |
| Acinetobacter harbinensis strain HITLi 7 | — | |
| Acinetobacter johnsonii 41886 Branch | — | |
| Acinetobacter johnsonii ANC 3681 | — | |
| Acinetobacter junii 41994 Branch | — | |
| Acinetobacter lwoffii WJ10621 | — | |
| Acinetobacter sp 41945 Branch | — | |
| Acinetobacter sp 41674 Branch | — | |
| Acinetobacter sp 41698 Branch | — | |
| Acinetobacter sp ETR1 | — | |
| Acinetobacter sp NIPH 298 | — | |
| Acinetobacter tandoii 41859 Branch | — | |
| Acinetobacter tjernbergiae 41962 Branch | — | |
| Acinetobacter towneri 41848 Branch | — | |
| Acinetobacter venetianus VE C3 | — | |
| Actinobacterium LLX17 | — | |
| Aeromonas bestiarum strain CECT 4227 | CECT 4227 | CECT |
| Aeromonas caviae strain CECT 4221 | CECT 4221 | CECT |
| Aeromonas hydrophila 4AK4 | — | |
| Aeromonas media 37528 Branch | — | |
| Aeromonas media strain | — | |
| ARB 37524 Branch | | |
| Aeromonas salmonicida | — | |
| subsp 37538 Branch | | |
| Aeromonas sp ZOR0002 | — | |
| Agrobacterium 22298 Branch | — | |
| Agrobacterium 22301 Branch | — | |
| Agrobacterium 22313 Branch | — | |
| Agrobacterium 22314 Branch | — | |
| Agrobacterium sp ATCC 31749 | ATCC 31749 | ATCC |
| Agrobacterium tumefaciens 22306 Branch | | |
| Agrobacterium tumefaciens strain MEJ076 | — | |
| Agrobacterium tumefaciens strain S2 | — | |
| Alkanindiges illinoisensis DSM 15370 | DSM 15370 | WFCC |
| alpha proteobacterium L41A | — | |
| Arthrobacter 20515 Branch | — | |
| Arthrobacter arilaitensis Re117 | — | |
| Arthrobacter chlorophenolicus A6 | — | |
| Arthrobacter nicotinovorans | — | |
| 20547 Branch | | |
| Arthrobacter phenanthrenivorans Sphe3 | — | |
| Arthrobacter sp 20511 Branch | — | |
| Arthrobacter sp PAO19 | — | |
| Arthrobacter sp W1 | — | |
| Aureimonas sp. Leaf427 | — | |
| Aureobasidium pullulans | — | |
| Bacillaceae Family 24 4101 12691 Branch | — | |
| Bacillus sp. LL01 | — | |
| Bacillus 12637 Branch | — | |
| Bacillus aerophilus strain C772 | — | |
| Bacillus thuringiensis | — | |
| serovar 12940 Branch | | |
| Brevundimonas nasdae strain TPW30 | — | |
| Brevundimonas sp 23867 Branch | — | |
| Brevundimonas sp EAKA | — | |
| Buchnera aphidicola str 28655 Branch | — | |
| Burkholderiales Order 15 | — | |
| 6136 Node 25777 | | |
| Buttiauxella agrestis 35837 Branch | — | |
| Candidatus Burkholderia verschuerenii | — | |
| Carnobacterium 5833 Branch | — | |
| Carnobacterium maltaromaticum | ATCC 35586 | ATCC |
| ATCC 35586 | | |
| Chryseobacterium 285 Branch | — | |
| Chryseobacterium daeguense DSM 19388 | DSM 19388 | WFCC |
| Chryseobacterium formosense | — | |
| Chryseobacterium sp YR005 | — | |
| Clavibacter 20772 Branch | — | |
| Clostridium diolis DSM 15410 | DSM 15410 | WFCC |
| Comamonas sp B 9 | — | |
| Curtobacterium flaccumfaciens | — | |
| 20762 Branch | | |
| Curtobacterium flaccumfaciens | — | |
| UCD AKU | | |
| Curtobacterium sp UNCCL17 | — | |
| Deinococcus aquatilis DSM 23025 | DSM 23025 | WFCC |
| Debaromyces hansenii | ATCC 36239 | ATCC |
| Duganella zoogloeoides | ATCC 25935 | |
| Dyadobacter 575 Branch | — | |
| Elizabethkingia anophelis | — | |
| Empedobacter falsenii strain 282 | — | |
| Enterobacter sp 638 | — | |
| Enterobacteriaceae Family 9 | — | |
| 3608 Node 35891 | | |
| Enterobacteriaceae Family 9 | — | |
| 593 Node 36513 | | |
| Epilithonimonas lactis | — | |
| Epilithonimonas tenax DSM 16811 | DSM 16811 | WFCC |
| Erwinia 35491 Branch | — | |
| Erwinia amylovora 35816 Branch | — | |
| Erwinia pyrifoliae 35813 Branch | — | |
| Erwinia tasmaniensis Et1 99 | DSM 17950 | WFCC |
| Escherichia coli ISC11 | — | |
| Exiguobacterium 13246 Branch | — | |
| Exiguobacterium 13260 Branch | — | |
| Exiguobacterium sibiricum 255 15 | DSM 17290 | WFCC |
| Exiguobacterium sp 13263 Branch | — | |
| Exiguobacterium undae 13250 Branch | — | |
| Exiguobacterium undae DSM 14481 | DSM 14481 | WFCC |
| Flavobacterium 237 Branch | — | |
| Flavobacterium aquatile LMG 4008 | LMG 4008 | WFCC |
| Flavobacterium chungangense | LMG 26729 | WFCC |
| LMG 26729 | | |
| Flavobacterium daejeonense DSM 17708 | DSM 17708 | WFCC |
| Flavobacterium hibernum | DSM 12611 | WFCC |
| strain DSM 12611 | | |
| Flavobacterium hydatis | — | |
| Flavobacterium johnsoniae UW101 | ATCC 17061D-5 | ATCC |
| Flavobacterium reichenbachii | — | |
| Flavobacterium soli DSM 19725 | DSM 19725 | WFCC |
| Flavobacterium sp 238 Branch | — | |
| Flavobacterium sp EM1321 | — | |
| Flavobacterium sp MEB061 | — | |
| Hanseniaspora uvarum | ATCC 18859 | |
| Hanseniaspora occidentalis | ATCC 32053 | |
| Herminiimonas arsenicoxydans | — | |
| Hymenobacter swuensis DY53 | — | |
| Janthinobacterium 25694 Branch | — | |
| Janthinobacterium agaricidamnosum | DSM 9628 | WFCC |
| NBRC 102515 | | |
| Janthinobacterium lividum strain RIT308 | — | |
| Janthinobacterium sp RA13 | — | |
| Kocuria 20614 Branch | — | |
| Kocuria rhizophila 20623 Branch | — | |
| Lactobacillus acetotolerans | — | |
| Lactobacillus brevis | — | |
| Lactobacillus buchneri | — | |
| Lactobacillus futsaii | — | |
| Lactobacillus kefiranofaciens | — | |
| Lactobacillus panis | — | |
| Lactobacillus parafarraginis | — | |
| Lactobacillus plantarum | — | |
| Lactobacillus rapi | — | |
| Lactobacillus crispatus 5565 Branch | — | |
| Lactobacillus plantarum WJL | — | |
| Lactobacillus reuteri 5515 Branch | — | |
| Leuconostoc mesenteroides | ATCC 8293 | |
| Luteibacter sp 9135 | — | |
| Massilia timonae CCUG 45783 | — | |
| Methylobacterium extorquens | — | |
| 23001 Branch | | |
| Methylobacterium sp 22185 Branch | — | |
| Methylobacterium sp 285MFTsu5 1 | — | |
| Methylobacterium sp 88A | — | |
| Methylotenera versatilis 7 | — | |
| Microbacterium laevaniformans OR221 | — | |
| Microbacterium oleivorans | — | |
| Microbacterium sp MEJ108Y | — | |
| Microbacterium sp UCD TDU | — | |
| Microbacterium testaceum StLB037 | — | |
| Micrococcus luteus strain RIT304 | NCTC 2665 | NCTC |
| Mycobacterium abscessus 19573 Branch | — | |
| Neosartorya fischeri | — | |
| Oxalobacteraceae bacterium AB 14 | — | |
| Paenibacillus sp FSL 28088 Branch | — | |
| Paenibacillus sp FSL H7 689 | — | |
| Pantoea sp. SL1 M5 | — | |
| Pantoea 36041 Branch | — | |
| Pantoea agglomerans strain 4 | — | |
| Pantoea agglomerans strain 4 | — | |
| Pantoea agglomerans strain LMAE 2 | — | |
| Pantoea agglomerans Tx10 | — | |
| Pantoea sp 36061 Branch | — | |
| Pantoea sp MBLJ3 | — | |
| Pantoea sp SL1 M5 | — | |
| Paracoccus sp PAMC 22219 | — | |
| Patulibacter minatonensis DSM 18081 | DSM 18081 | WFCC |
| Pectobacterium carotovorum | — | |
| subsp carotovorum | | |
| strain 28625 Branch | | |
| Pediococcus ethanolidurans | — | |
| Pediococcus pentosaceus | ATCC 33314 | |
| Pedobacter 611 Branch | — | |
| Pedobacter agri PB92 | — | |
| Pedobacter borealis DSM 19626 | DSM 19626 | WFCC |
| Pedobacter kyungheensis | — | |
| strain KACC 16221 | | |
| Pedobacter sp R20 19 | — | |
| Periglandula ipomoeae | — | |
| Planomicrobium glaciei CHR43 | — | |
| Propionibacterium acnes | — | |
| Propionibacterium 20955 Branch | — | |
| Propionibacterium acnes 21065 Branch | — | |
| Pseudomonas fluorescens | — | |
| Pseudomonas sp. DSM 29167 | — | |
| Pseudomonas sp. Leaf15 | — | |
| Pseudomonas syringae | — | |
| Pseudomonas 39524 Branch | — | |
| Pseudomonas 39642 Branch | — | |
| Pseudomonas 39733 Branch | — | |
| Pseudomonas 39744 Branch | — | |
| Pseudomonas 39791 Branch | — | |
| Pseudomonas 39821 Branch | — | |
| Pseudomonas 39834 Branch | — | |
| Pseudomonas 39875 Branch | — | |
| Pseudomonas 39880 Branch | — | |
| Pseudomonas 39889 Branch | — | |
| Pseudomonas 39894 Branch | — | |
| Pseudomonas 39913 Branch | — | |
| Pseudomonas 39931 Branch | — | |
| Pseudomonas 39942 Branch | — | |
| Pseudomonas 39979 Branch | — | |
| Pseudomonas 39996 Branch | — | |
| Pseudomonas 40058 Branch | — | |
| Pseudomonas 40185 Branch | — | |
| Pseudomonas abietaniphila strain KF717 | — | |
| Pseudomonas chlororaphis strain EA105 | — | |
| Pseudomonas cremoricolorata | DSM 17059 | WFCC |
| DSM 17059 | | |
| Pseudomonas entomophila L48 | — | |
| Pseudomonas extremaustralis | — | |
| 14 3 substr 14 3b | | |
| Pseudomonas fluorescens BBc6R8 | — | |
| Pseudomonas fluorescens BS2 | ATCC 12633 | ATCC |
| Pseudomonas fluorescens EGD AQ6 | — | |
| Pseudomonas fluorescens | — | |
| strain AU 39831 Branch | | |
| Pseudomonas fluorescens strain AU10973 | — | |
| Pseudomonas fluorescens strain AU14440 | — | |
| Pseudomonas fragi B25 | NCTC 10689 | NCTC |
| Pseudomonas frederiksbergensis strain SI8 | — | |
| Pseudomonas fulva strain MEJ086 | — | |
| Pseudomonas fuscovaginae 39768 Branch | — | |
| Pseudomonas gingeri NCPPB 3146 | NCPPB 3146 | NCPPB |
| Pseudomonas lutea | — | |
| Pseudomonas luteola XLDN4 9 | — | |
| Pseudomonas mandelii JR 1 | — | |
| Pseudomonas moraviensis R28 S | — | |
| Pseudomonas mosselii SJ10 | — | |
| Pseudomonas plecoglossicida | — | |
| NB 39639 Branch | | |
| Pseudomonas poae RE*1 1 14 | — | |
| Pseudomonas pseudoalcaligenes AD6 | — | |
| Pseudomonas psychrophila HA 4 | — | |
| Pseudomonas putida DOT T1E | — | |
| Pseudomonas putida strain KF703 | — | |
| Pseudomonas putida strain MC4 5222 | — | |
| Pseudomonas rhizosphaerae | — | |
| Pseudomonas rhodesiae strain FF9 | — | |
| Pseudomonas sp 39813 Branch | — | |
| Pseudomonas simiae strain 2 36 | — | |
| Pseudomonas simiae strain MEB105 | — | |
| Pseudomonas sp 11 12A | — | |
| Pseudomonas sp 2 922010 | — | |
| Pseudomonas sp CF149 | — | |
| Pseudomonas sp Eur1 9 41 | — | |
| Pseudomonas sp LAMO17WK12 I2 | — | |
| Pseudomonas sp PAMC 25886 | — | |
| Pseudomonas sp PTA1 | — | |
| Pseudomonas sp R62 | — | |
| Pseudomonas sp WCS374 | — | |
| Pseudomonas synxantha BG33R | — | |
| Pseudomonas synxantha BG33R | — | |
| Pseudomonas syringae 39550 Branch | — | |
| Pseudomonas syringae 39596 Branch | — | |
| Pseudomonas syringae 40123 Branch | — | |
| Pseudomonas syringae CC 39499 Branch | — | |
| Pseudomonas syringae | — | |
| pv panici str LMG 2367 | | |
| Pseudomonas syringae strain mixed | — | |
| Pseudomonas tolaasii 39796 Branch | — | |
| Pseudomonas tolaasii PMS117 | — | |
| Pseudomonas veronii 1YdBTEX2 | — | |
| Pseudomonas viridiflava CC1582 | — | |
| Pseudomonas viridiflava strain LMCA8 | — | |
| Pseudomonas viridiflava TA043 | — | |
| Pseudomonas viridiflava UASWS0038 | — | |
| Rahnella 35969 Branch | — | |
| Rahnella 35970 Branch | — | |
| Rahnella 35971 Branch | — | |
| Rahnella aquatilis HX2 | — | |
| Rahnella sp WP5 | — | |
| Raoultella ornithinolytica | — | |
| Rhizobiales Order 22324 Branch | — | |
| Rhizobium sp YR528 | — | |
| Rhodococcus fascians A76 | — | |
| Rhodococcus sp BS 15 | — | |
| Saccharomyces cerevisiae | — | |
| Sanguibacter keddieii | DSM 10542 | WFCC |
| Serratia fonticola AU 35657 Branch | — | |
| Serratia fonticola AU AP2C | — | |
| Serratia liquefaciens ATCC 27592 | ATCC 27592 | ATCC |
| Serratia sp H 35589 Branch | — | |
| Shewanella 37294 Branch | — | |
| Shewanella baltica 37301 Branch | — | |
| Shewanella baltica 37315 Branch | — | |
| Shewanella baltica OS 37308 Branch | — | |
| Shewanella baltica OS 37312 Branch | — | |
| Shewanella baltica OS185 | — | |
| Shewanella baltica OS223 | — | |
| Shewanella baltica OS678 | — | |
| Shewanella oneidensis MR 1 | — | |
| Shewanella putrefaciens HRCR 6 | — | |
| Shewanella sp W3 18 1 | — | |
| Sphingobacterium sp ML3W | — | |
| Sphingobium japonicum BiD32 | — | |
| Sphingobium xenophagum 24443 Branch | — | |
| Sphingomonas echinoides ATCC 14820 | ATCC 14820 | ATCC |
| Sphingomonas parapaucimobilis | ATCC 51231 | ATCC |
| NBRC 15100 | | |
| Sphingomonas paucimobilis NBRC 13935 | ATCC 29837 | ATCC |
| Sphingomonas phyllosphaerae 5 2 | — | |
| Sphingomonas sp 23777 Branch | — | |
| Sphingomonas sp STIS6 2 | — | |
| Staphylococcus 6317 Branch | — | |
| Staphylococcus equorum UMC CNS 924 | — | |
| Staphylococcus sp 6275 Branch | — | |
| Staphylococcus sp 6240 Branch | — | |
| Staphylococcus sp OJ82 | — | |
| Staphylococcus xylosus strain LSR 02N | — | |
| Stenotrophomonas 14028 Branch | — | |
| Stenotrophomonas 42816 Branch | — | |
| Stenotrophomonas maltophilia | — | |
| 42817 Branch | | |
| Stenotrophomonas maltophilia | — | |
| PML 168 | | |
| Stenotrophomonas maltophilia | — | |
| strain ZBG7B | | |
| Stenotrophomonas rhizophila | — | |
| Stenotrophomonas sp RIT309 | — | |
| Streptococcus gallolyticus subsp | — | |
| gallolyticus TX20005 | | |
| Streptococcus infantarius subsp | — | |
| infantarius 2242 Branch | | |
| Streptococcus infantarius subsp | ATCC BAA 102 | ATCC |
| infantarius ATCC BAA 102 | | |
| Streptococcus macedonicus ACA DC 198 | ATCC BAA-249 | ATCC |
| Streptomyces olindensis | — | |
| Variovorax paradoxus 110B | — | |
| Variovorax paradoxus ZNC0006 | — | |
| Variovorax sp CF313 | — | |
| Vibrio fluvialis 44473 Branch | — | |
| Xanthomonas campestris 37936 Branch | — | |
| Xanthomonas campestris pv raphani 756C | — | |

In some embodiments, the compositions disclosed herein comprise a microorganism listed in Table 2. These microbes are characterized in WO2020051379A1, which is incorporated by reference in its entirety.

TABLE 2
Strains

| Strain Number | Genus | Species |
|---|---|---|
| DP1 | Pseudomonas | fluorescens |
| DP2 | Hanseniaspora | occidentalis |
| DP3 | Leuconostoc | mesenteroides |
| DP4 | Aureobasidium | pullulans |
| DP5 | Debaromyces | hansenii |
| DP6 | Bacillus | wiedmannii |
| DP7 | Pichia | fermentans |
| DP8 | Hanseniaspora | opuntiae |
| DP9 | Pediococcus | pentosaceus |
| DP10 | Bacillus | velezensis |
| DP11 | Pseudomonas | putida |
| DP12 | Microbacterium | sp. |
| DP13 | Bacillus | mycoides |
| DP14 | Arthrobacter | luteolus |
| DP15 | Curtobacterium | sp. |
| DP16 | Lacihabitans | lacunae |
| DP17 | Rahnella | aquatilis |
| DP18 | Pseudomonas | sp. |
| DP19 | Curtobacterium | pusillum |
| DP20 | Stenotrophomonas | rhizophila |
| DP21 | Candida | santamariae |
| DP22 | Rahnella | sp. |
| DP23 | Erwinia | billingiae |
| DP24 | Filobasidium | globisporum |
| DP25 | Penicillium | solitum |
| DP26 | Methylobacterium | sp. |
| DP27 | Sphingomonas | sp. |
| DP28 | Aureobasidium | pullulans |
| DP29 | Pseudoclavibacter | helvolus |
| DP30 | Microbacterium | testaceum |
| DP31 | Sporisorium | reilianum |
| DP32 | Hafnia | paralvei |
| DP33 | Erwinia | persicinus |
| DP34 | Plantibacter | flavus |
| DP35 | Pantoea | ananatis |
| DP36 | Pantoea | vagans |
| DP37 | Pseudomonas | rhodesiae |
| DP38 | Rhodococcus | sp. |
| DP39 | Agrobacterium | tumefaciens |
| DP40 | Pantoea | sp. |
| DP41 | Corynebacterium | mucifaciens |
| DP42 | Pseudomonas | lundensis |
| DP43 | Janthinobacterium | sp. |
| DP44 | Herbaspirillum | sp. |
| DP45 | Sanguibacter | keddieii |
| DP46 | Pantoea | agglomerans |
| DP47 | Cronobacter | dublinensis |
| DP48 | Bacillus | paralicheniformis |
| DP49 | Bacillus | gibsonii |
| DP50 | Enterobacter | sp. |
| DP51 | Klebsiella | aerogenes |
| DP52 | Arthrobacter | sp. |
| DP53 | Pseudomonas | fragi |
| DP54 | Methylobacterium | adhaesivum |
| DP55 | Bacillus | megaterium |
| DP56 | Paenibacillus | lautus |
| DP57 | Bacillus | mycoides |
| DP58 | Janthinobacterium | svalbardensis |
| DP59 | Kosakonia | cowanii |
| DP60 | Bacillus | simplex |
| DP61 | Lelliottia | sp. |
| DP62 | Erwinia | sp. |
| DP63 | Pseudomonas | azotoformans |
| DP64 | Hanseniaspora | uvarum |
| DP65 | Bacillus | sp. |
| DP66 | Hanseniaspora | occidentalis |
| DP67 | Bacillus | sp. |
| DP68 | Bacillus | atrophaeus |
| DP69 | Bacillus | sp. |
| DP70 | Bacillus | subtilis |
| DP71 | Rhodotorula | sp. |
| DP72 | Bacillus | zhangzhouensis |
| DP73 | Bacillus | clausii |
| DP74 | Bacillus | coagulans |
| DP75 | Pseudomonas | gessardii |
| DP76 | Ochrobactrum | sp. |
| DP77 | Bacillus | aryabhattai |
| DP78 | Erwinia | rhapontici |
| DP79 | Pseudomonas | fragi |
| DP80 | Methylobacterium | adhaesivum |
| DP81 | Bacillus | clausii |
| DP82 | Bacillus | clausii |
| DP83 | Bacillus | clausii |
| DP84 | Microbacterium | sp. |
| DP85 | Methanolacinia | petrolearia |
| DP86 | Bacillus | velezensis |
| DP87 | Lactobacillus | plantarum |
| DP88 | Bacillus | velezensis |
| DP89 | Bacillus | subtilis |
| DP90 | Lactobacillus | plantarum |
| DP92 | Bacillus | subtilis |
| DP93 | Leuconostoc | mesenteroides |
| DP94 | Lactobacillus | brevis |
| DP95 | Lactobacillus | paracasei |
| DP96 | Lactobacillus | casei |
| DP97 | Lactococcus | garvieae |
| DP98 | Lactococcus | garvieae |
| DP99 | Weissella | cibaria |
| DP100 | Lactobacillus | plantarum |
| DP101 | Pediococcus | pentosaceus |
| DP102 | Pichia | kudriavzevii |

In some embodiments, species of interest are isolated from plant-based food sources normally consumed raw. These isolated compositions of microorganisms from individual plant sources can be combined to create a new mixture of organisms. Particular species from individual plant sources can be selected and mixed with other species cultured from other plant sources, which have been similarly isolated and grown. In some embodiments, species of interest are grown in pure cultures before being prepared for consumption or administration. In some embodiments, the species of interest are grown by bulk fermentation. In certain embodiments, the species of interest are grown in fermenters under controlled conditions of temperature, pH, aeration/gas flow and/or agitation. In some embodiments, the organisms grown in pure culture are combined to form a synthetic combination of organisms.

In some embodiments, the microbial composition comprises proteobacteria or gamma proteobacteria. In some embodiments, at least one species from each of four groups is present, the four groups being: lactic acid bacteria, Bacilli, proteobacteria, and yeast. In some embodiments, at least one additional microbe from a group other than the four stated above is also present. In some embodiments, the microbial composition comprises several species of Pseudomonas. In some embodiments, species from another genus are also present. In some embodiments, a species from the genus Duganella is also present. In some embodiments of said microbial composition, the population comprises at least three unique isolates selected from the group consisting of Pseudomonas, Acinetobacter, Aeromonas, Curtobacterium, Escherichia, Lactobacillus, Serratia, Streptococcus, and Stenotrophomonas. In some embodiments, the bacteria are selected based upon their ability to degrade fibers, including plant fibers, and/or to modulate production of one or more branch chain fatty acids, short chain fatty acids, and/or flavones in a mammalian gut.

In some embodiments, microbial compositions comprise isolates that are capable of modulating production or activity of the enzymes involved in fatty acid metabolism, such as acetolactate synthase I, N-acetylglutamate synthase, acetate kinase, Acetyl-CoA synthetase, acetyl-CoA hydrolase, Glucan 1,4-alpha-glucosidase, or Bile acid symporter Acr3.

In some embodiments, microbial compositions of the disclosure comprise microbial strains that are capable of producing one or more neurotransmitters, e.g., neurotransmitters selected from the group consisting of serotonin, gamma-aminobutyric acid (GABA), dopamine, acetylcholine and combinations thereof. In some embodiments, the microbial compositions of the disclosure are capable of producing one or more neurotransmitters, e.g., neurotransmitters selected from the group consisting of serotonin, GABA, dopamine, acetylcholine and combinations thereof. In some embodiments, compositions of the disclosure comprise a metabolite (e.g., a neurotransmitter) produced by one or more microbial strains of the microbial composition.

In some embodiments, the administered microbial compositions colonize the treated mammal's digestive tract. In some embodiments, these colonizing microbes comprise bacterial assemblages present in whole food plant-based diets. In some embodiments, these colonizing microbes comprise Pseudomonas with a diverse species denomination that is present and abundant in whole food plant-based diets. In some embodiments, these colonizing microbes reduce free fatty acids absorbed into the body of a host by absorbing the free fatty acids in the gastrointestinal tract of mammals. In some embodiments, these colonizing microbes comprise genes encoding metabolic functions related to desirable health outcomes such as increased bone mineral density, prevention of loss of bone mineral density, improved bone turnover markers, improved inflammatory metabolic indicators, improvement of symptoms of menopause, perimenopause, or postmenopause, increased vitamin K production, etc.

Some embodiments comprise bacteria that are not completely viable but act by releasing metabolites that act in the gastro-intestinal tract of a patient promoting bone health or other desirable outcome. Some embodiments comprise a prebiotic composition derived from metabolites present in whole food plant-based materials, identified and enriched as part of the formula for oral delivery.

In some embodiments, biological materials (such as microbial strains including bacteria and fungi) are refrigerated at temperatures of about −20° C. or at about −80° C., e.g., with use of laboratory freezers. In some embodiments, biological materials are refrigerated at temperatures of about 4° C. In some embodiments, biological materials are stored using the vapor phase of liquid nitrogen that brings the temperature to −170° C. In some embodiments, biological materials are lyophilized.

In some embodiments, the constituent microbial strains of an assemblage are grown or produced separately and stored below room temperature (e.g. at about 4° C., about −20° C., about −80° C., or about −170° C.) prior to mixing. In some embodiments, the constituent microbial strains are stored in this manner for about 1 day to about 5 years, for example, for about 1 day to 1 week, about 1 week to 2 weeks, about 1 week to 1 month, about 1 month to 3 months, about 3 months to 6 months, about 6 months to 1 year, or about 1 year to 5 years. In some embodiments, the constituent microbial strains of an assemblage are combined into a composition or formulation, optionally in combination with other components (e.g. prebiotic components), and the composition or formulation is stored below room temperature (e.g. at about 4° C., about −20° C., about −80° C., or about −170° C.) prior to administration. In some embodiments, the composition or formulation is stored in this manner for about 1 day to about 5 years, for example, for about 1 day to 1 week, about 1 week to 2 weeks, about 1 week to 1 month, about 1 month to 3 months, about 3 months to 6 months, about 6 months to 1 year, or about 1 year to 5 years. In some embodiments, the foregoing storage conditions prevent or reduce a loss of viability or potency of the microbial strains.

In embodiments, compositions of the disclosure comprise dietary supplements, medical food compositions, and/or pharmaceutical compositions.

### Prebiotics

Prebiotics, in accordance with the teachings of this disclosure, comprise compositions that promote the growth of beneficial bacteria in the intestines. Prebiotic substances can be consumed by a relevant probiotic, or otherwise assist in keeping the relevant probiotic alive or stimulate its growth. When consumed in an effective amount, prebiotics also beneficially affect a subject's naturally-occurring gastrointestinal microflora and thereby impart health benefits apart from just nutrition. Prebiotic foods enter the colon and serve as substrate for the endogenous microbes, thereby indirectly providing the host with energy, metabolic substrates, and essential micronutrients. The body's digestion and absorption of prebiotic foods is dependent upon microbial metabolic activity, which salvages energy for the host from nutrients that escaped digestion and absorption in the small intestine.

Prebiotics help probiotics flourish in the gastrointestinal tract, and accordingly, their health benefits largely are indirect. Metabolites generated by colonic fermentation by intestinal microflora, such as short-chain fatty acids, can play important functional roles in the health of the host. Prebiotics can be useful agents for enhancing the ability of intestinal microflora to provide benefits to their host.

Prebiotics, in accordance with the embodiments of this disclosure, include, without limitation, mucopolysaccharides, oligosaccharides, polysaccharides, amino acids, vitamins, nutrient precursors, proteins, and combinations thereof.

According to particular embodiments, compositions comprise a prebiotic comprising a dietary fiber, including, without limitation, polysaccharides and oligosaccharides. These compounds have the ability to increase the number of probiotics, and augment their associated benefits. For example, an increase of beneficial Bifidobacteria likely changes the intestinal pH to support the increase of Bifidobacteria, thereby decreasing pathogenic organisms.

Non-limiting examples of oligosaccharides that are categorized as prebiotics in accordance with particular embodiments include fructooligosaccharides (i.e. oligofructose), inulins, isomalto-oligosaccharides, lactilol, lactosucrose, lactulose, pyrodextrins, soy oligosaccharides, transgalacto-oligosaccharides, cellulose, and xylo-oligosaccharides.

According to particular embodiments, compositions comprise a prebiotic comprising one or more amino acids.

Prebiotics are found naturally in a variety of foods including, without limitation, cabbage, bananas, berries (e.g., blueberries), asparagus, garlic, wheat, oats, barley (and other whole grains), flaxseed, tomatoes, Jerusalem artichoke, onions and chicory, greens (e.g., dandelion greens, spinach, collard greens, chard, kale, mustard greens, turnip greens), and legumes (e.g., lentils, kidney beans, chickpeas, navy beans, white beans, black beans). Accordingly, in some embodiments, the composition comprises a prebiotic comprising a substance derived from one or more of the foregoing foods. In some embodiments, the substance is derived from berries, e.g., blueberries. In some embodiments, the prebiotic is a liquid, juice, or extract derived from a fruit or vegetable. In some embodiments, preparing and/or processing the prebiotic comprises a filtration step. In some embodiments, the prebiotic comprises a dried substance derived from a fruit or vegetable. In some embodiments, the prebiotic comprises a dried (and optionally, ground) fruit and/or vegetable powder. In some embodiments, the prebiotic comprises a dried (and optionally, ground) berry powder. In certain embodiments, the prebiotic comprises a dried (and optionally, ground) blueberry powder. In some embodiments, the prebiotic is processed or prepared via dehydration. In some embodiments, the prebiotic is processed or prepared via freeze-drying or lyophilization.

In some embodiments, the prebiotic may comprise a substrate that enables the production of a metabolite. For example, in certain embodiments, the prebiotic comprises a substance that enables the production of a short chain fatty acid (e.g. acetate, propionate, or butyrate). For example, in some embodiments, the prebiotic comprises a substance that enables the production of acetate. In some embodiments, prebiotics comprise one or more beneficial compounds such as flavonoids, anti-cyanines, phytoestrogens, and resberetrol.

According to particular embodiments, compositions comprise a prebiotic present in a sweetener composition or functional sweetened composition in an amount sufficient to promote health and wellness.

In particular embodiments, prebiotics also can be added to high-potency sweeteners or sweetened compositions. Non-limiting examples of prebiotics that can be used in this manner include fructooligosaccharides (i.e. oligofructose), xylooligosaccharides, galactooligosaccharides, and combinations thereof.

Many prebiotics have been discovered from dietary intake including, but not limited to: antimicrobial peptides, polyphenols, Okara (soybean pulp by product from the manufacturing of tofu), polydextrose, lactosucrose, malto-oligosaccharides, gluco-oligosaccharides (GOS), fructo-oligosaccharides (FOS), xantho-oligosaccharides, and soluble dietary fiber in general. Types of soluble dietary fiber include, but are not limited to, psyllium, pectin, or inulin. Phytoestrogens (plant-derived isoflavone compounds that have estrogenic effects) have been found to have beneficial growth effects of intestinal microbiota through increasing microbial activity and microbial metabolism by increasing the blood testosterone levels, in humans and farm animals. Phytoestrogen compounds include but are not limited to: Oestradiol, Daidzein, Formononetin, Biochainin A, Genistein, and Equol. Accordingly, in some embodiments, prebiotics comprising soluble dietary fiber and/or phytoestrogens (e.g. dried (optionally, ground) berry powder, e.g. blueberry powder) are beneficial when used in a composition of the disclosure.

Dosage for the compositions described herein are deemed to be “effective doses,” indicating that the probiotic or prebiotic composition is administered in a sufficient quantity to alter the physiology of a subject in a desired manner. In some embodiments, the desired alterations include reducing, preventing, treating, or managing osteoporosis or osteopenia and sequelae associated with these conditions. In some embodiments, the desired alterations include reducing, preventing, treating, or managing inflammation. In some embodiments, the desired alterations occur in a menopausal, perimenopausal or postmenopausal subject. In some embodiments, the desired alterations include reducing or managing the severity of one or more symptoms of menopause, perimenopause or postmenopause.

Vitamin K is found in many fruits and vegetables including broccoli, grapes, lettuce, and olives and plays a role in a wide range of biological activities including calcium metabolism, cell proliferation, oxidative stress, and inflammation. Vitamin K2 (menaquinone) plays a vital role in bone synthesis and is produced by bacteria residing in the gastrointestinal tract. Vitamin K2 affects the proliferation and differentiation of osteoblasts, leading to increased osteoblast activity and bone matrix production. Specifically, Vitamin K2 stimulates the expression of osteoprotegerin (OPG) and inhibits the expression of receptor activator of nuclear factor kappa-B ligand (RANKL) on osteoblasts, leading to increased proliferation and activation. Vitamin K2 has also been shown to inhibit osteoclastic bone resorption, preventing the breakdown of bone.

In some embodiments, the compositions of the disclosure improve Vitamin K2 absorption. In some embodiments, the compositions of the disclosure produce Vitamin K2 in the gut of a subject. In some embodiments, the microbes of the disclosure are selected based upon their having genes involved in biosynthetic pathways for producing Vitamin K2.

In some embodiments, administering the compositions of the disclosure to a subject alters the microbiome of the subject, as compared to a suitable control. In some embodiments, the alteration is an increase in abundance of a microbial strain which is administered as part of the composition. In some embodiments, administering the compositions of the disclosure to a subject increases the abundance of genes involved in metabolic pathways for producing Vitamin K in the microbiome of the subject, as compared to a suitable control. In some embodiments, the suitable control is the microbiome of an appropriate control subject or control group that is not administered the composition. In some embodiments, the suitable control is a historical control, e.g., the microbiome of the subject prior to the first administration of the composition. In some embodiments, genes involved in the biosynthesis of menaquinol-6, menaquinol-9, menaquinol-10, demethylmenaquinol-6, and demethylmenaquinol-9 are increased in abundance following administration of a composition of the disclosure. Analysis of a subject's microbiome and any alterations thereto may be accomplished by any suitable technique known in the art, e.g., via metagenomic sequencing of an appropriate sample, e.g., a stool sample.

In some embodiments, the composition comprises a cryoprotectant. In general, a cryoprotectant functions through work by dissolving in water, lowering the melting point or a composition containing cells, and preventing or limiting intracellular and extracellular crystals from forming in cells during a freezing process. A cryoprotectant can allow for preservation of strain viability for prolonged periods of time, including extending viability for years. In some embodiments, the cryoprotectant is a prebiotic. In some embodiments, the cryoprotectant includes glycerol, trehalose, or Dimethyl sulfoxide (DMSO). In some embodiments, the cryoprotectant is derived from a plant source. In some embodiments, viability, measured at room temperature, is increased for at least one microbe by addition of cryoprotectant to a composition comprising said microbe wherein the composition is stored frozen. In some embodiments, viability is increased by at least 10, 15, 25, 35, 45, 50, 55, 65, 75, 85, 95, or 100 percent. Typically, a cryoprotectant (e.g., glycerol, trehalose, or DMSO) concentration of about 5% to 15% (e.g., about 5% to 12.5%, about 5% to 10%, about 5% to 7.5%, about 7.5% to 15%, about 7.5% to 12.5%, about 7.5% to 10%, about 10% to 15%, about 10% to 12.5%, or about 12.5% to 15%) is used and permits survival of a substantial fraction of isolated cells after freezing and thawing from cryogenic temperatures. One skilled in the art will recognize a cryoprotectant formulation can adjusted dependent on the cellular species to be preserved. For example, certain species (e.g., gamma proteobacteria) are sensitive to cryopreservation and lose considerable viability after few days in cryo-storage.

### Methods of Use

Included within the scope of this disclosure are methods for treatment and/or management of musculoskeletal disorders including osteoporosis, osteopenia, Paget's disease, stunting, osteoarthritis, osteomyelitis, and delayed or non-union fractures. Also included within the scope of this disclosure are methods for preventing or prophylactically treating one of the aforementioned musculoskeletal disorders, e.g. by administering a composition of the disclosure to a subject at risk for having or developing one of said disorders. Also included within the scope of the disclosure are methods for managing, preventing, or reducing inflammation. Also included within the scope of the disclosure are methods for managing, treating, or preventing a symptom of menopause, perimenopause or postmenopause, e.g. a vasomotor symptom.

These methods include treatment with a prebiotic composition (e.g., a composition comprising or consisting of dried fruit or vegetable powder, FOS, GOS, and/or other appropriate polysaccharide), optionally in conjunction with a probiotic composition as described herein, one or more digestible saccharides (e.g., lactose, glucose, or galactose), a buffer, or a combination thereof. These methods optionally are used in combination with other treatments to treat, manage or prevent the disease, disorder, or condition. Any suitable treatment can be used. In some embodiments the additional treatment is administered before, during, or after treatment with a prebiotic composition, or any combination thereof. In an embodiment, when the disease or disorder (e.g., a musculoskeletal disorder) is not completely or substantially completely eliminated by treatment with a prebiotic composition, the additional treatment is administered after prebiotic treatment is terminated. The additional treatment is used on an as-needed basis.

In certain embodiments, the methods include additional treatment with vitamin D. In certain embodiments, a composition of the disclosure (e.g., a DMA composition as described herein) and vitamin D are administered to a subject simultaneously. In certain embodiments, a composition of the disclosure (e.g., a DMA composition as described herein) and vitamin D are administered to a subject separately. In certain embodiments, a composition of the disclosure (e.g., a DMA composition as described herein) and vitamin D are administered to a subject sequentially. In certain embodiments, vitamin D is administered to a subject prior to administration of a composition of the disclosure (e.g., a DMA composition as described herein). In certain embodiments, vitamin D is administered to a subject following administration of a composition of the disclosure (e.g., a DMA composition as described herein).

In some embodiments, a subject to be treated or prophylactically treated for a musculoskeletal disorder (e.g. osteoporosis or osteopenia), inflammation, and/or one or more symptoms of perimenopause, menopause, or postmenopause is a human. In an embodiment, the human subject is a preterm newborn, a full term newborn, an infant up to one year of age, a young child (e.g., 1 yr to 12 yrs), a teenager (e.g., 13-19 yrs), an adult (e.g., 20-64 yrs), a pregnant women, an adult in perimenopause, an adult in menopause, an adult in postmenopause, an adult that has undergone a menopause transition, or an elderly adult (65 yrs and older). In some embodiments, about 1 year to about 6 years have passed since the subject's last menstruation. In some embodiments, about 1, about 2, about 3, about 4, about 5, or about 6 years have passed since the subject's last menstruation. In some embodiments, more than about 6 years, more than about 7 years, more than about 8 years, more than about 9 years, or more than about 10 years have elapsed since the subject's last menstruation. In some embodiments, the subject has a bone density T-score equal to or less than about −2.5. In some embodiments, the subject has a bone density T-score equal to or greater than about −2.49. In some embodiments, the subject has been diagnosed with a musculoskeletal disorder, e.g. osteoporosis or osteopenia. In some embodiments, the subject is at risk for a musculoskeletal disorder, e.g. osteoporosis or osteopenia.

In some embodiments, the condition to be treated is osteoporosis or osteopenia. In some embodiments, the condition to be treated is osteoporosis or osteopenia, and treating osteoporosis further involves administration of any one or combination of known anti-osteoporosis medications or treatments. These include, but are not limited to, bisphosphonates (alendronate, risedronate, ibandronate, zolendronate), biologics (denosumab, romosozumab), selective estrogen receptor mediators (Raloxifene), or anabolic agents (teriparatide, abaloparatide).

In some embodiments, the compositions and methods of the disclosure may be used to lessen a decrease in, maintain, or improve bone health in a subject, e.g., as compared to a suitable control. In certain embodiments, the suitable control is a control group or a control subject that is not administered the composition. In certain embodiments, the suitable control is a historical control, e.g., the bone health in the subject prior to the first administration of the composition, or the rate of change (e.g., decline) in bone health in the subject prior to the first administration of the composition. Bone health may be assessed using any appropriate technique known in the art, including, e.g., by bone mineral density (BMD) or by trabecular bone score (TBS).

In some embodiments, the compositions and methods of the disclosure may be used to lessen a decrease in, maintain, or improve bone mineral density (BMD) in a subject as compared to a suitable control. In certain embodiments, the suitable control is a control group or a control subject that is not administered the composition. In certain embodiments, the suitable control is a historical control, e.g., the BMD in the subject prior to the first administration of the composition, or the rate of change (e.g., decline) in BMD in the subject prior to the first administration of the composition. In some embodiments, total-body BMD, lumbar spine BMD, femur BMD, femoral neck BMD, hip BMD, or any combination thereof are maintained or improved as compared to a suitable control. BMD may be measured by any appropriate technique known in the art, e.g. by dual energy X-ray absorptiometry.

In some embodiments, BMD is measured prior to the first administration of a composition of the invention to establish a “baseline”, and BMD is measured again in the same subject following one or more repeated administrations of said composition. In some embodiments, BMD is measured after about, e.g., 2, 4, 6, 8, 10, 12, 14, 16, 18, 20, 22 or 24 months of regular administration (e.g., daily, twice daily, thrice daily or four-times daily administration) of a composition of the disclosure to the subject. In a particular embodiment, BMD is measured about 6 months and/or about 12 months after the first administration. In some embodiments, following repeated administration of a composition of the disclosure, BMD is improved relative to a control group which is not administered the composition. In some embodiments, following regular administration of a composition of the disclosure, BMD is maintained at about the pre-administration baseline or is improved relative to the pre-administration baseline. In some embodiments, BMD in a subject decreases following administration of a composition of the disclosure, and the rate of decrease is slower than the rate of decrease in the subject prior to the first administration of the composition. In some embodiments, BMD in a subject decreases following administration of a composition of the disclosure, and the decrease is less severe than would have occurred without administration of the composition. In some embodiments, BMD is improved relative to the pre-administration baseline or relative to a control group by at least about 0.5%, 1%, 2%, 3%, 4%, 5%, 6%, 8%, 10%, 15%, 20%, 25%, 30%, 40%, 50%, 60%, 70%, 80%, 90%, or at least about 100%. For example, in some embodiments, BMD of the subject decreases following administration of a composition of the disclosure, and the decrease is less severe than that of a control group which is not administered the composition, thereby representing an improvement over the control group.

In some embodiments, the compositions and methods of the disclosure may be used to lessen a decrease in, maintain, or improve areal bone mineral density (aBMD) in a subject as compared to a suitable control. In certain embodiments, the suitable control is a control group or a control subject that is not administered the composition. In certain embodiments, the suitable control is a historical control, e.g., the aBMD in the subject prior to the first administration of the composition, or the rate of change (e.g., decline) in aBMD in the subject prior to the first administration of the composition. In some embodiments, total body aBMD, lumbar spine aBMD, femur aBMD, femoral neck aBMD, hip aBMD, or any combination thereof are maintained or improved as compared to a suitable control. aBMD can be measured by any appropriate technique known in the art, e.g. by dual energy X-ray (DXA) absorptiometry.

In some embodiments, aBMD is measured prior to the first administration of a composition of the invention to establish a “baseline”, and aBMD is measured again in the same subject following one or more repeated administrations of said composition. In some embodiments, aBMD is measured after about, e.g., 2, 4, 6, 8, 10, 12, 14, 16, 18, 20, 22 or 24 months of regular administration (e.g., daily, twice daily, thrice daily or four times daily administration) of a composition of the disclosure to the subject. In a particular embodiment, aBMD is measured about 6 months and/or about 12 months after the first administration. In some embodiments, following repeated administration of a composition of the disclosure, aBMD is improved relative to a control group which is not administered a composition of the disclosure. In some embodiments, following regular administration of a composition of the disclosure, aBMD is maintained at about the pre-administration baseline or is improved relative to the pre-administration baseline. In some embodiments, aBMD is improved relative to the pre-administration baseline or relative to a control group by at least about 0.5%, 1%, 2%, 3%, 4%, 5%, 6%, 8%, 10%, 15%, 20%, 25%, 30%, 40%, 50%, 60%, 70%, 80%, 90%, or at least about 100%. For example, in some embodiments, aBMD in the subject decreases following administration of a composition of the disclosure, and the decrease is less severe than that of a control group which is not administered the composition, thereby representing an improvement over the control group. In some embodiments, aBMD in a subject decreases following administration of a composition of the disclosure, and the decrease is less severe than would have occurred without administration of the composition. In some embodiments, aBMD in a subject decreases following administration of a composition of the disclosure, and the rate of decrease is slower than the rate of decrease in the subject prior to the first administration of the composition.

In some embodiments, the compositions and methods of the disclosure may be used to lessen a decrease in, maintain, or improve volumetric bone mineral density (vBMD) (e.g., lumbar spine vBMD) in a subject as compared to a suitable control. In certain embodiments, the suitable control is a control group or a control subject that is not administered the composition. In certain embodiments, the suitable control is a historical control, e.g., the vBMD in the subject prior to the first administration of the composition, or the rate of change (e.g., decline) in vBMD in the subject prior to the first administration of the composition. vBMD differs from areal BMD (aBMD): aBMD is measured by a DXA scan which allows for the measurement of bone density on a two-dimensional axis, whereas vBMD is measured by a CT scan, which allows for the measurement of the actual volume of bone in a subject's scan. vBMD can be measured by any appropriate technique known in the art, e.g. by quantitative computed tomography.

In some embodiments, vBMD is measured prior to the first administration of a composition of the invention to establish a “baseline”, and vBMD is measured again in the same subject following one or more repeated administrations of said composition. In some embodiments, vBMD is measured after about, e.g., 2, 4, 6, 8, 10, 12, 14, 16, 18, 20, 22 or 24 months of regular administration (e.g., daily, twice daily, thrice daily or four times daily administration) of a composition of the disclosure to the subject. In a particular embodiment, vBMD is measured about 6 months and/or about 12 months after the first administration. In some embodiments, vBMD is improved relative to a control group which is not administered a composition of the disclosure. In some embodiments, following repeated administration of a composition of the disclosure, vBMD is maintained at about the pre-administration baseline or is improved relative to the pre-administration baseline. In some embodiments, vBMD is improved relative to the pre-administration baseline or relative to the control group by at least about 0.5%, 1%, 2%, 3%, 4%, 5%, 6%, 8%, 10%, 15%, 20%, 25%, 30%, 40%, 50%, 60%, 70%, 80%, 90%, or at least about 100%. For example, in some embodiments, vBMD in the subject decreases following administration of a composition of the disclosure, and the decrease is less severe than that of a control group which is not administered the composition, thereby representing an improvement over the control group. In some embodiments, vBMD in a subject decreases following administration of a composition of the disclosure, and the decrease is less severe than would have occurred without administration of the composition. In some embodiments, vBMD in a subject decreases following administration of a composition of the disclosure, and the rate of decrease is slower than the rate of decrease in the subject prior to the first administration of the composition.

In some embodiments, compositions and methods of the disclosure may be used to improve BMD, vBMD, and/or aBMD in a subject by about 0.5% to 100% relative to a suitable control, e.g., relative to a pre-administration baseline or relative to a control group. For example, in some embodiments, BMD, vBMD, and/or BMD are improved by about 0.5% to 100%, about 0.5% to 90%, about 0.5% to 80%, about 0.5% to 70%, about 0.5% to 60%, about 0.5% to 50%, about 0.5% to 40%, about 0.5% to 30%, about 0.5% to 25%, about 0.5% to 20%, about 0.5% to 15%, about 0.5% to 10%, about 0.5% to 8%, about 0.5% to 6%, about 0.5% to 5%, about 0.5% to 4%, about 0.5% to 3%, about 0.5% to 2%, about 0.5% to 1%, about 1% to 100%, about 1% to 90%, about 1% to 80%, about 1% to 70%, about 1% to 60%, about 1% to 50%, about 1% to 40%, about 1% to 30%, about 1% to 25%, about 1% to 20%, about 1% to 15%, about 1% to 10%, about 1% to 8%, about 1% to 6%, about 1% to 5%, about 1% to 4%, about 1% to 3%, about 1% to 2%, about 2% to 100%, about 2% to 90%, about 2% to 80%, about 2% to 70%, about 2% to 60%, about 2% to 50%, about 2% to 40%, about 2% to 30%, about 2% to 25%, about 2% to 20%, about 2% to 15%, about 2% to 10%, about 2% to 8%, about 2% to 6%, about 2% to 5%, about 2% to 4%, about 2% to 3%, about 3% to 100%, about 3% to 90%, about 3% to 80%, about 3% to 70%, about 3% to 60%, about 3% to 50%, about 3% to 40%, about 3% to 30%, about 3% to 25%, about 3% to 20%, about 3% to 15%, about 3% to 10%, about 3% to 8%, about 3% to 6%, about 3% to 5%, about 3% to 4%, about 4% to 100%, about 4% to 90%, about 4% to 80%, about 4% to 70%, about 4% to 60%, about 4% to 50%, about 4% to 40%, about 4% to 30%, about 4% to 25%, about 4% to 20%, about 4% to 15%, about 4% to 10%, about 4% to 8%, about 4% to 6%, about 4% to 5%, about 5% to 100%, about 5% to 90%, about 5% to 80%, about 5% to 70%, about 5% to 60%, about 5% to 50%, about 5% to 40%, about 5% to 30%, about 5% to 25%, about 5% to 20%, about 5% to 15%, about 5% to 10%, about 5% to 8%, about 5% to 6%, about 6% to 100%, about 6% to 90%, about 6% to 80%, about 6% to 70%, about 6% to 60%, about 6% to 50%, about 6% to 40%, about 6% to 30%, about 6% to 25%, about 6% to 20%, about 6% to 15%, about 6% to 10%, about 6% to 8%, about 8% to 100%, about 8% to 90%, about 8% to 80%, about 8% to 70%, about 8% to 60%, about 8% to 50%, about 8% to 40%, about 8% to 30%, about 8% to 25%, about 8% to 20%, about 8% to 15%, about 8% to 10%, about 10% to 100%, about 10% to 90%, about 10% to 80%, about 10% to 70%, about 10% to 60%, about 10% to 50%, about 10% to 40%, about 10% to 30%, about 10% to 25%, about 10% to 20%, about 10% to 15%, about 15% to 100%, about 15% to 90%, about 15% to 80%, about 15% to 70%, about 15% to 60%, about 15% to 50%, about 15% to 40%, about 15% to 30%, about 15% to 25%, about 15% to 20%, about 20% to 100%, about 20% to 90%, about 20% to 80%, about 20% to 70%, about 20% to 60%, about 20% to 50%, about 20% to 40%, about 20% to 30%, about 20% to 25%, about 25% to 100%, about 25% to 90%, about 25% to 80%, about 25% to 70%, about 25% to 60%, about 25% to 50%, about 25% to 40%, about 25% to 30%, about 30% to 100%, about 30% to 90%, about 30% to 80%, about 30% to 70%, about 30% to 60%, about 30% to 50%, about 30% to 40%, about 40% to 100%, about 40% to 90%, about 40% to 80%, about 40% to 70%, about 40% to 60%, about 40% to 50%, about 50% to 100%, about 50% to 90%, about 50% to 80%, about 50% to 70%, about 50% to 60%, about 60% to 100%, about 60% to 90%, about 60% to 80%, about 60% to 70%, about 70% to 100%, about 70% to 90%, about 70% to 80%, about 80% to 100%, about 80% to 90%, or about 90% to 100%, relative to a suitable control.

In some embodiments, the compositions and methods of the disclosure may be used to lessen a decrease in, maintain, alter, or improve (TBS). TBS is an analytical tool, based on the use of standard dual-energy X-ray absorptiometry images, which may be used to evaluate bone texture and which provides information about bone microarchitecture (see Harvey et al. (2015) Bone 78:216-224). TBS can provide information about bone health that is not captured by BMD. For example, TBS may be able to differentiate between two three-dimensional bone microarchitectures with the same BMD if they have different trabecular characteristics (e.g. trabecular number/separation and/or connectivity density). Without wishing to be bound by theory, an elevated TBS may correlate with stronger, better-connected, more fracture-resistant microarchitectures. By contrast, a lower TBS may correlate with weaker and more fracture-prone microarchitectures.

In some embodiments, administration of the compositions of the disclosure can be used to lessen a decrease in, maintain, alter, or increase TBS in a subject (e.g. lumbar TBS) as compared to a suitable control. In certain embodiments, the suitable control is a control group or a control subject that is not administered the composition, e.g., a control group that is administered a placebo. In certain embodiments, the suitable control is a historical control, e.g., the TBS in the subject prior to the first administration of the composition, or the rate of change (e.g., decline) in TBS in the subject prior to the first administration of the composition.

In certain embodiments, TBS is evaluated prior to the first administration of a composition of the invention to establish a “baseline”, and TBS is measured again in the same subject following one or more repeated administrations of said composition. In some embodiments, TBS is measured again after about, e.g., 2, 4, 6, 8, 10, 12, 14, 16, 18, 20, 22 or 24 months of regular administration (e.g., daily, twice daily, thrice daily or four times daily administration) of a composition of the disclosure to the subject. In a particular embodiment, TBS is measured about 6 months and/or about 12 months after the first administration. In some embodiments, TBS is altered or improved relative to a control group which is not administered a composition of the disclosure. In some embodiments, following regular administration of a composition of the disclosure, TBS does not substantially decrease relative to the pre-administration baseline. In some embodiments, TBS is improved relative to the pre-administration baseline or relative to the control group by at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 8%, 10%, 15%, 20%, 25%, 30%, 40% or at least about 50%. For example, in some embodiments, TBS in the subject decreases following administration of a composition of the disclosure, and the decrease is less severe than that of a control group which is not administered the composition, thereby representing an improvement over the control group. In some embodiments, TBS in a subject decreases following administration of a composition of the disclosure, and the decrease is less severe than would have occurred without administration of the composition. In some embodiments, TBS in a subject decreases following administration of a composition of the disclosure, and the rate of decrease is slower than the rate of decrease in the subject prior to the first administration of the composition.

In some embodiments, compositions and methods of the disclosure may be used to improve TBS in a subject by about 0.01% to 50% relative to a suitable control, e.g., relative to a pre-administration baseline or relative to a control group. For example, in some embodiments, TBS is improved by about 0.01% to 50%, about 0.01% to 40%, about 0.01% to 30%, about 0.01% to 25%, about 0.01% to 20%, about 0.01% to 15%, about 0.01% to 10%, about 0.01% to 5%, about 0.01% to 1%, about 0.01% to 0.5%, about 0.01% to 0.1%, about 0.01% to 0.05%, about 0.05% to 50%, about 0.05% to 40%, about 0.05% to 30%, about 0.05% to 25%, about 0.05% to 20%, about 0.05% to 15%, about 0.05% to 10%, about 0.05% to 5%, about 0.05% to 1%, about 0.05% to 0.5%, about 0.05% to 0.1%, about 0.1% to 50%, about 0.1% to 40%, about 0.1% to 30%, about 0.1% to 25%, about 0.1% to 20%, about 0.1% to 15%, about 0.1% to 10%, about 0.1% to 5%, about 0.1% to 1%, about 0.1% to 0.5%, about 0.5% to 50%, about 0.5% to 40%, about 0.5% to 30%, about 0.5% to 25%, about 0.5% to 20%, about 0.5% to 15%, about 0.5% to 10%, about 0.5% to 5%, about 0.5% to 1%, about 1% to 50%, about 1% to 40%, about 1% to 30%, about 1% to 25%, about 1% to 20%, about 1% to 15%, about 1% to 10%, about 1% to 5%, about 5% to 50%, about 5% to 40%, about 5% to 30%, about 5% to 25%, about 5% to 20%, about 5% to 15%, about 5% to 10%, about 10% to 50%, about 10% to 40%, about 10% to 30%, about 10% to 25%, about 10% to 20%, about 10% to 15%, about 15% to 50%, about 15% to 40%, about 15% to 30%, about 15% to 25%, about 15% to 20%, about 20% to 50%, about 20% to 40%, about 20% to 30%, about 20% to 25%, about 25% to 50%, about 25% to 40%, about 25% to 30%, about 30% to 50%, about 30% to 40%, or about 40% to 50%, relative to a suitable control.

In some embodiments, the compositions and methods of the disclosure may be used to alter levels of one or more biochemical markers of bone turnover (i.e. bone turnover markers (BTM)) in a subject as compared to a suitable control. In some embodiments, the BTM is a marker of bone formation, e.g. procollagen type 1 N-terminal propeptide (PINP). In other embodiments, the BTM is a marker of bone resorption, e.g. C-terminal telopeptide of type I collagen (CTX). In some embodiments, both a marker of bone formation and a marker of bone resorption are measured, either simultaneously or at different times. In some embodiments, levels of one or more BTMs are measured in the subject's blood, serum, plasma, or urine. BTMs can be measured using any appropriate technique known in the art (see, e.g., Szulc et al., Osteoporos Int. (2017)), for example by ELISA, ECLIA, or CLIA.

In some embodiments, administration of the compositions of the disclosure can alter or increase levels of a bone formation marker in the subject as compared to a suitable control. In some embodiments, the suitable control is a control group or a control subject that is not administered the composition, e.g., a control group that is administered a placebo. In certain embodiments, the suitable control is a historical control, e.g., the level(s) of one or more BTM in the subject prior to the first administration of the composition.

In some embodiments, the levels of one or more BTMs in a subject are evaluated prior to the first administration of a composition of the disclosure to establish a “baseline”, and are measured again in the same subject following one or more repeated administrations of said composition. In some embodiments, the levels of the one or more BTMs are re-measured after about, e.g., 0.5, 1, 2, 4, 6, 8, 10, 12, 14, 16, 18, 20, 22 or 24 months of regular administration (e.g., daily, twice daily, thrice daily or four times daily administration) of a composition of the disclosure to the subject. In a particular embodiment, the levels of the one or more BTM levels are re-measured about 6 months and/or about 12 months after the first administration. In some embodiments, the level of a bone formation marker (e.g. PINP) changes relative to the pre-administration baseline by at least about 5%, 10%, 15%, 20%, 25%, 30%, 35%, 40%, 45%, 50%, 60%, 70%, 80%, 90%, 100%, 125%, 150%, 175%, 100%, 200%, 300%, 400% or 500%. In certain embodiments, the change in the level of the bone formation marker is an increase relative to the pre-administration baseline. In some embodiments, the level of a bone resorption marker (e.g. CTX) changes relative to the pre-administration baseline by at least about 5%, 10%, 15%, 20%, 25%, 30%, 35%, 40%, 45%, 50%, 60%, 70%, 80%, 90%, 100%, 125%, 150%, 175%, 100%, 200%, 300%, 400%, or 500%. In certain embodiments, the change in the level of the bone resorption marker is a decrease relative to the pre-administration baseline. In some embodiments, the change in the level of the bone formation and/or bone resorption marker is relative to a control group which is not administered a composition of the disclosure.

In some embodiments, compositions and methods of the disclosure may be used to alter levels of one or more BTM in a subject by about 5% to 500% relative to a suitable control, e.g., relative to a pre-administration baseline or relative to a control group. For example, in some embodiments, the levels of one or more BTM are independently increased or decreased by about 5% to 500%, about 5% to 400%, about 5% to 300%, about 5% to 200%, about 5% to 150%, about 5% to 100%, about 5% to 75%, about 5% to 50%, about 5% to 40%, about 5% to 30%, about 5% to 25%, about 5% to 20%, about 5% to 15%, about 5% to 10%, about 10% to 500%, about 10% to 400%, about 10% to 300%, about 10% to 200%, about 10% to 150%, about 10% to 100%, about 10% to 75%, about 10% to 50%, about 10% to 40%, about 10% to 30%, about 10% to 25%, about 10% to 20%, about 10% to 15%, about 15% to 500%, about 15% to 400%, about 15% to 300%, about 15% to 200%, about 15% to 150%, about 15% to 100%, about 15% to 75%, about 15% to 50%, about 15% to 40%, about 15% to 30%, about 15% to 25%, about 15% to 20%, about 20% to 500%, about 20% to 400%, about 20% to 300%, about 20% to 200%, about 20% to 150%, about 20% to 100%, about 20% to 75%, about 20% to 50%, about 20% to 40%, about 20% to 30%, about 20% to 25%, about 25% to 500%, about 25% to 400%, about 25% to 300%, about 25% to 200%, about 25% to 150%, about 25% to 100%, about 25% to 75%, about 25% to 50%, about 25% to 40%, about 25% to 30%, about 30% to 500%, about 30% to 400%, about 30% to 300%, about 30% to 200%, about 30% to 150%, about 30% to 100%, about 30% to 75%, about 30% to 50%, about 30% to 40%, about 40% to 500%, about 40% to 400%, about 40% to 300%, about 40% to 200%, about 40% to 150%, about 40% to 100%, about 40% to 75%, about 40% to 50%, about 50% to 500%, about 50% to 400%, about 50% to 300%, about 50% to 200%, about 50% to 150%, about 50% to 100%, about 50% to 75%, about 75% to 500%, about 75% to 400%, about 75% to 300%, about 75% to 200%, about 75% to 150%, about 75% to 100%, about 100% to 500%, about 100% to 400%, about 100% to 300%, about 100% to 200%, about 100% to 150%, about 150% to 500%, about 150% to 400%, about 150% to 300%, about 150% to 200%, about 200% to 500%, about 200% to 400%, about 200% to 300%, about 300% to 500%, about 300% to 400%, or about 400% to 500%, relative to a suitable control.

Changes in levels of hormones during perimenopause, menopause, and postmenopause, and namely a reduction in estrogen levels, can lead to inflammation. Markers of osteoporosis or osteopenia can include elevated levels of inflammatory cytokines in the blood including, but not limited to: Tumor necrosis factor alpha (TNFα), Interleukin-17 (IL-17), Interleukin-4 (IL-4), Interferon gamma (IFNγ), and Receptor activator of nuclear factor kappa-B ligand (RANKL). In some embodiments, the compositions disclosed herein are rationally designed for the production of SCFAs, which have been identified as anti-inflammatory mediators. Accordingly, in some embodiments, the compositions and methods of the disclosure may be used to decrease inflammation.

In certain embodiments, the compositions and methods may be used to alter or decrease circulating levels of one or more inflammatory cytokines and/or markers of inflammation, including, e.g., C-reactive protein (CRP), IL-17, TNF, IL-1B, RANKL, IFNγ, or any combination thereof, as compared to a suitable control. In some embodiments, the suitable control is a control group or a control subject that is not administered the composition, e.g., a control group that is administered a placebo. In certain embodiments, the suitable control is a historical control, e.g., the level(s) of one or more inflammatory cytokines and/or markers of inflammation in the subject prior to the first administration of the composition. In some embodiments, levels of one or more inflammatory cytokines and/or markers of inflammation are measured in the subject's blood, serum, or plasma.

In certain embodiments, the levels of one or more inflammatory cytokines and/or markers of inflammation in a subject are evaluated prior to the first administration of a composition of the disclosure to establish a “baseline”, and then said inflammatory cytokines/markers are measured again in the same subject following one or more repeated administrations of the composition. In some embodiments, the levels of the one or more inflammatory cytokines and/or markers of inflammation are re-measured after about, e.g., 0.5, 1, 2, 4, 6, 8, 10, 12, 14, 16, 18, 20, 22 or 24 months of regular administration (e.g., daily, twice daily, thrice daily or four times daily administration) of a composition of the disclosure to the subject. In a particular embodiment, the levels of the one or more inflammatory cytokines and/or markers of inflammation are re-measured about 6 months and/or about 12 months after the first administration. In some embodiments, the level of an inflammatory cytokine and/or marker of inflammation (e.g. CRP) decreases relative to the pre-administration baseline by at least about 5%, 10%, 15%, 20%, 25%, 30%, 35%, 40%, 45%, 50%, 60%, 70%, 80%, 90%, 100%, 125%, 150%, 175%, 100%, 200%, 300%, 400%, 500%, 1000%, 2500%, 5000%, or at least about 10000%. In some embodiments, the changes in levels of one or more inflammatory cytokines and/or markers of inflammation are relative to a control group which is not administered a composition of the disclosure. Inflammatory cytokines and/or markers of inflammation may be measured using any appropriate technique known in the art, e.g. by ELISA or by multiplex array.

In some embodiments, compositions and methods of the disclosure may be used to decrease the level of one or more inflammatory cytokine(s) and/or marker(s) of inflammation in a subject by about 5% to 10000% relative to a suitable control, e.g., relative to a pre-administration baseline or relative to a control group. For example, in some embodiments, the level of an inflammatory cytokine and/or marker of inflammation (e.g., CRP, IL-17, TNF, IL-1B, RANKL, or IFNγ) is decreased by about 5% to 10000%, about 5% to 5000%, about 5% to 2500%, about 5% to 1000%, about 5% to 500%, about 5% to 400%, about 5% to 300%, about 5% to 200%, about 5% to 100%, about 5% to 75%, about 5% to 50%, about 5% to 40%, about 5% to 30%, about 5% to 25%, about 5% to 20%, about 5% to 15%, about 5% to 10%, about 10% to 10000%, about 10% to 5000%, about 10% to 2500%, about 10% to 1000%, about 10% to 500%, about 10% to 400%, about 10% to 300%, about 10% to 200%, about 10% to 100%, about 10% to 75%, about 10% to 50%, about 10% to 40%, about 10% to 30%, about 10% to 25%, about 10% to 20%, about 10% to 15%, about 15% to 10000%, about 15% to 5000%, about 15% to 2500%, about 15% to 1000%, about 15% to 500%, about 15% to 400%, about 15% to 300%, about 15% to 200%, about 15% to 100%, about 15% to 75%, about 15% to 50%, about 15% to 40%, about 15% to 30%, about 15% to 25%, about 15% to 20%, about 20% to 10000%, about 20% to 5000%, about 20% to 2500%, about 20% to 1000%, about 20% to 500%, about 20% to 400%, about 20% to 300%, about 20% to 200%, about 20% to 100%, about 20% to 75%, about 20% to 50%, about 20% to 40%, about 20% to 30%, about 20% to 25%, about 25% to 10000%, about 25% to 5000%, about 25% to 2500%, about 25% to 1000%, about 25% to 500%, about 25% to 400%, about 25% to 300%, about 25% to 200%, about 25% to 100%, about 25% to 75%, about 25% to 50%, about 25% to 40%, about 25% to 30%, about 30% to 10000%, about 30% to 5000%, about 30% to 2500%, about 30% to 1000%, about 30% to 500%, about 30% to 400%, about 30% to 300%, about 30% to 200%, about 30% to 100%, about 30% to 75%, about 30% to 50%, about 30% to 40%, about 40% to 10000%, about 40% to 5000%, about 40% to 2500%, about 40% to 1000%, about 40% to 500%, about 40% to 400%, about 40% to 300%, about 40% to 200%, about 40% to 100%, about 40% to 75%, about 40% to 50%, about 50% to 10000%, about 50% to 5000%, about 50% to 2500%, about 50% to 1000%, about 50% to 500%, about 50% to 400%, about 50% to 300%, about 50% to 200%, about 50% to 100%, about 50% to 75%, about 75% to 10000%, about 75% to 5000%, about 75% to 2500%, about 75% to 1000%, about 75% to 500%, about 75% to 400%, about 75% to 300%, about 75% to 200%, about about 75% to 100%, about 100% to 10000%, about 100% to 5000%, about 100% to 2500%, about 100% to 1000%, about 100% to 500%, about 100% to 400%, about 100% to 300%, about 100% to 200%, about 200% to 10000%, about 200% to 5000%, about 200% to 2500%, about 200% to 1000%, about 200% to 500%, about 200% to 400%, about 200% to 300%, about 300% to 10000%, about 300% to 5000%, about 300% to 2500%, about 300% to 1000%, about 300% to 500%, about 300% to 400%, about 400% to 10000%, about 400% to 5000%, about 400% to 2500%, about 400% to 1000%, about 400% to 500%, about 500% to 10000%, about 500% to 5000%, about 500% to 2500%, about 500% to 1000%, about 1000% to 10000%, about 1000% to 5000%, about 1000% to 2500%, about 2500% to 10000%, about 2500% to 5000%, or about 5000% to 10000%, relative to a suitable control.

In some embodiments, the condition to be treated is osteoarthritis. In some embodiments, the condition to be treated is osteoarthritis, and treating the condition further involves administration of any one or combination of known anti-osteoarthritis medications or treatments. In some embodiments, a composition of the disclosure (e.g., a DMA composition as described herein) is administered to a subject simultaneously with a treatment or medication for osteoarthritis. In some embodiments, a composition of the disclosure (e.g., a DMA composition as described herein) and a treatment or medication for osteoarthritis are administered to a subject separately. In some embodiments, a composition of the disclosure (e.g., a DMA composition as described herein) and a treatment or medication for osteoarthritis are administered to a subject sequentially. In some embodiments, a treatment or medication for osteoarthritis is administered to a subject prior to administration of a composition of the disclosure (e.g., a DMA composition as described herein). In certain embodiments, a treatment or medication for osteoarthritis is administered to a subject following administration of a composition of the disclosure (e.g., a DMA composition as described herein).

Treatments and medications for osteoarthritis include, but are not limited to, surgery, analgesics, non-steroidal anti-inflammatory drugs (aspirin, celecoxib, diclofenac, diflunisal, etodolac, ibuprofen, indomethacin, ketoprofen, ketorolac, nabumetone, naproxen, oxaprozin, piroxicam), menthol, weight loss regimens, physical exercise, acupuncture, narcotics (Codeine, Fentanyl, Hydrocodone, hydroporphone, meperidine, methadone, oxycodone), and physical therapy.

In some embodiments, the condition to be treated is a delayed or non-union fracture. In an embodiment, the condition to be treated is a delayed or non-union fracture, and treating the condition further involves administration of any one or combination of known treatments to improve delayed or non-union fractures. These include, but are not limited to surgical bone grafts or fixations and bone stimulation.

In some embodiments, the condition to be treated is osteomyelitis. The methods disclosed herein, optionally, are used in combination with other treatments to treat or prevent osteomyelitis. In some embodiments, a composition of the disclosure (e.g., a DMA composition as described herein) is administered to a subject simultaneously with a treatment for osteomyelitis. In some embodiments, a composition of the disclosure (e.g., a DMA composition as described herein) and a treatment for osteomyelitis are administered to a subject separately. In some embodiments, a composition of the disclosure (e.g., a DMA composition as described herein) and a treatment for osteomyelitis are administered to a subject sequentially. In some embodiments, a treatment for osteomyelitis is administered to a subject prior to administration of a composition of the disclosure (e.g., a DMA composition as described herein). In certain embodiments, a treatment for osteomyelitis is administered to a subject following administration of a composition of the disclosure (e.g., a DMA composition as described herein). Typical treatments for osteomyelitis include, but are not limited to, intravenous or oral antibiotics (clindamycin, cefotetan, ticarcillin/clavulanate, ceftriaxone, metronidazole, piperacillin/tazobactam, fluoroquinolone, cefepime, ciprofloxacin, imipenem/cilastin, vancomycin, trimethoprim/sulfamethoxazole, minocycline, nafcillin, oxacillin, cefazolin, penicillin) and surgery. Any suitable treatment for osteomyelitis can be used. These include, but are not limited to, removal of diseased tissue and antibiotics, administered either orally or intravenously.

In some embodiments, the condition to be treated or prevented is a symptom of menopause, perimenopause or postmenopause. In some embodiments, the symptom of menopause, perimenopause or postmenopause is hot flushes, sweating, episodes of sweating, night sweats, heart discomfort, unusual awareness of heart beat, heart skipping, heart racing, heart tightness, depressive mood, feeling down, feeling sad, feeling on verge of tears, lack of drive, mood swings, irritability, feeling nervous, inner tension, feeling aggressive, anxiety, inner restlessness, feeling panicky, physical exhaustion, mental exhaustion, general decrease in performance, impaired memory, decrease in concentration, forgetfulness, sexual problems, change in sexual desire, change in sexual activity, change in sexual satisfaction, bladder problems, difficulty in urinating, increased need to urinate, bladder incontinence, dryness of the vagina, sensation of dryness or burning in the vagina, difficulty with sexual intercourse, joint and muscular discomfort, pain in the joints or rheumatoid arthritis. In some embodiments, the symptom is a vasomotor symptom, e.g. hot flushes or sweating. In some embodiments, the presence and/or severity of a symptom of menopause in the subject is improved, as compared to a suitable control. In some embodiments, the suitable control is a control group or a control subject that is not administered the composition, e.g., a control group that is administered a placebo. In certain embodiments, the suitable control is a historical control, e.g., the presence or severity of the symptom of menopause in the subject prior to the first administration of the composition.

In some embodiments, the presence and severity of menopause symptoms are evaluated prior to the first administration of a composition of the disclosure to establish a “baseline”, and then said symptoms are evaluated again following one or more repeated administrations of the composition. In some embodiments, the severity of the menopause, perimenopause, or postmenopause symptoms are re-measured after about, e.g., 0.5, 1, 2, 4, 6, 8, 10, 12, 14, 16, 18, 20, 22 or 24 months of regular administration (e.g., daily, twice daily, thrice daily or four times daily administration) of a composition of the disclosure to the subject. In a particular embodiment, the presence and severity of the one or more menopause, perimenopause, or postmenopause symptoms are re-evaluated about 1 month, about 2 months, about 4 months, about 6 months, about 8 months, about 10 months, and/or about 12 months after the first administration. In some embodiments, the presence and severity of the one or more menopause, perimenopause, or postmenopause symptoms are measured by the Menopause Rating Scale (MRS; see Heinemann et al. (2004) Health Qual Life Outcomes 2:45). In some embodiments, the severity of the symptom of menopause as measured by the MRS decreases by 1 to 4 points, 1 to 3 points, 1 to 2 points, 2 to 4 points, 2 to 3 points, or 3 to 4 points, as compared to a suitable control. In some embodiments, the severity of the menopause symptom as measured by the MRS decreases by 1, 2, 3 or 4 points, as compared to a suitable control. In a particular embodiment, the severity of hot flushes and/or sweating will decrease by 1, 2, 3 or 4 points as measured by the MRS, as compared to a suitable control.

### Timing and Dose of Probiotics and Prebiotics

In certain embodiments, probiotic microbes (such as one or more Lactobacillus species, one or more Leuconostoc species, one or more Pichia species, or any combination of the foregoing) are given prior to beginning treatment with a prebiotic. In certain embodiments, probiotic microbes (such as one or more Lactobacillus species, one or more Leuconostoc species, one or more Pichia species, or any combination of the foregoing) are given in conjunction with treatment with a prebiotic (e.g., comprising or consisting essentially of dried fruit or vegetable powder, FOS, GOS, and/or other appropriate polysaccharide), for part or all of the treatment with the prebiotic. Thus, in certain embodiments, some or all doses of a prebiotic (e.g., comprising or consisting essentially of dried fruit or vegetable powder, FOS, GOS, and/or other appropriate polysaccharide) are accompanied by a dose of microbes, e.g., live cultured microbes, e.g., one or more Lactobacillus species, one or more Leuconostoc species, one or more Pichia species, or any combination of the foregoing. In an embodiment, microbes (e.g., one or more Lactobacillus species, one or more Leuconostoc species, one or more Pichia species, or any combination of the foregoing) are given initially with a prebiotic (e.g., comprising or consisting essentially of dried fruit or vegetable powder, FOS, GOS, and/or other appropriate polysaccharide), but then use of the microbes is discontinued. For example, the initial one, two, three, four, five, six, seven, eight, nine, ten or more than ten days of treatment with a prebiotic (e.g., comprising or consisting essentially of dried fruit or vegetable powder, FOS, GOS, and/or other appropriate polysaccharide) further comprises doses of microbes, with the use of microbes discontinued after that time. In an embodiment, microbes, (e.g., bacteria in yogurt), or microbes by themselves, can be given for the first two days of treatment; then the administration of microbes is discontinued. In another embodiment, probiotic microbes, either alone or in combination with other substances or treatments are used after the treatment with a prebiotic (comprising or consisting essentially of dried fruit or vegetable powder, FOS, GOS, and/or other appropriate polysaccharide) is terminated. The microbes can be taken for any suitable period after the termination of treatment with prebiotic and can be taken daily or at regular or irregular intervals. Doses can be as described below. Any suitable amount of probiotic per serving can be used that allows an effective microbiota in the GI as demonstrated by an increase in bone mineral density, improved bone architecture, protection from loss of bone mineral density, improved bone turnover markers, or improvement in other markers of osteoporosis or osteopenia. Markers of osteoporosis or osteopenia can include elevated levels of Inflammatory cytokines in the blood including, but not limited to: Tumor necrosis factor alpha (TNFα), Interleukin-17 (IL-17), Interleukin-4 (IL-4), Interferon gamma (IFNγ), Receptor activator of nuclear factor kappa-B ligand (RANKL). They can also include increased one resorption blood markers (breakdown) crosslinked C-telopeptide of type 1 collagen (CTX), or decreased Bone formation blood markers: osteocalcin, alkaline phosphatase, N-terminal propeptide of type 1 collagen.

Any suitable amount of probiotic per serving can be used that allows an effective microbiota in the GI as demonstrated by an improvement in the disorder, disease, state, or condition to be treated. This may be, e.g., a decrease in inflammation or a reduction in severity of menopause/perimenopause/postmenopause symptoms. This may also be healthy bone healing, in which the improvement may be a decreased incidence of delayed or non-union fractures or increased normal fracture callus formation. Markers of fracture healing defects include delayed healing, non-union fracture healing, or changes in fracture callus architecture (including increased size or adiposity of the fracture callus).

Any suitable amount of probiotic per serving can be used that allows an effective microbiota in the GI as demonstrated by a decrease in inflammation. Inflammatory cytokines or markers of inflammation may include, for example, CRP, IL-17, TNF, IL-1B, IL-4, RANKL, and IFNγ.

Any suitable amount of probiotic per serving can be used that allows an effective microbiota in the GI as demonstrated by a decrease in severity in a symptom of perimenopause, menopause, or postmenopause. By way of a nonlimiting example, the symptom may be a vasomotor symptom, e.g. hot flushes or sweating.

Typically, probiotics are given as live cultured microbes. The dose can be, e.g., 0.001 mg to 1 mg, or 0.5 mg to 5 mg, or 1 mg to 1000 mg, or 2 mg to 200 mg, or 2 mg to 100 mg, or 2 mg to 50 mg, or 2 mg to 25 mg, or 2 mg to 20 mg, or 4 mg to 50 mg, or 4 mg to 25 mg, or 5 mg to 50 mg, or 5 mg to 40 mg, or 5 mg to 30 mg or 5 mg to 25 mg, or 5 mg to 20 mg, or 10 mg to 100 mg, or 10 mg to 75 mg, or 10 mg to 50 mg, or 10 mg to 25 mg, or 10 mg to 15 mg, or 20 mg to 100 mg, or 20 mg to 75 mg, or 20 mg to 50 mg, or 20 mg to 40 mg, or 20 mg to 30 mg, or 20 mg to 25 mg, or 50 mg to 200 mg, or 50 mg to 150 mg, or 50 mg to 100 mg, or 50 mg to 75 mg, or 100 mg to 1000 mg, or 100 mg to 800 mg, or 100 mg to 600 mg, or 100 mg to 500 mg, or 100 mg to 400 mg, or 100 mg to 300 mg, or 100 mg to 200 mg, or 100 mg to 150 mg, or 200 mg to 1000 mg, or about 10 mg, about 11 mg, about 12 mg, about 12.5 mg, about 13 mg, about 14 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 125 mg, about 150 mg, about 175 mg, or about 200 mg per serving or per day. In certain embodiments, Lactobacillus brevis is used in a dose of about 25 mg to about 50 mg per serving or per day. In certain embodiments, Lactobacillus plantarum is used in a dose of about 25 mg to about 50 mg per serving or per day. In certain embodiments, Leuconostoc mesenteroides is used in a dose of about 25 mg to about 50 mg per serving or per day. In certain embodiments, Pichia kudriavzevii is used in a dose of about 100 mg to about 200 mg per serving or per day. In certain embodiments, L. acidophilus is used in a dose of about 12.5 mg per serving or per day. The probiotic microbes can also be 0.5% w/w to 20% w/w of the final composition. The dose of probiotics can be given in combination with one or more prebiotics.

Another common way of specifying the amount of probiotics is as a colony forming unit (cfu). In an embodiment, one or more strains of probiotic microbes are ingested in an amount of between 1×10\hat{ }5 and 1×10\hat{ }13 cfu's per serving or per day. In some embodiments, one or more strains of probiotic microbes (e.g., Lactobacillus plantarum, Lactobacillus brevis, Leuconostoc mesenteroides, and/or Pichia kudriavzevii) are ingested in a serving or in a total daily dose of about 1×10\hat{ }5 cfu's to about 1×10\hat{ }13 cfu's, or about 1×10\hat{ }5 to about 1×10\hat{ }12 cfu's, or about 1×10\hat{ }5 cfu's to about 1×10\hat{ }11 cfu's, or about 1×10\hat{ }5 cfu's to about 3×10\hat{ }10 cfu's, or about 1×10\hat{ }5 cfu's to about 1.5×10\hat{ }10 cfu's, or about 1×10\hat{ }5 cfu's to about 1×10\hat{ }10 cfu's, or about 1×10\hat{ }5 cfu's to about 7.5×10\hat{ }9 cfu's, or about 1×10\hat{ }5 cfu's to about 5×10\hat{ }9 cfu's, or about 1×10\hat{ }5 cfu's to about 2.5×10\hat{ }9 cfu's, or about 1×10\hat{ }5 cfu's to about 1.25×10\hat{ }9 cfu's, or about 1×10\hat{ }5 cfu's to about 1×10\hat{ }9 cfu's, or about 1×10\hat{ }5 cfu's to about 1×10\hat{ }8 cfu's, or about 1×10\hat{ }5 cfu's to about 1×10\hat{ }7 cfu's, or about 1×10\hat{ }5 cfu's to about 1×10\hat{ }6 cfu's, or about 1×10\hat{ }6 to about 1×10\hat{ }13 cfu's, or about 1×10\hat{ }6 cfu's to about 1×10\hat{ }12 cfu's, or about 1×10\hat{ }6 cfu's to about 1×10\hat{ }11 cfu's, or about 1×10\hat{ }6 cfu's to about 3×10\hat{ }10 cfu's, or about 1×10\hat{ }6 cfu's to about 1.5×10\hat{ }10 cfu's, or about 1×10\hat{ }6 cfu's to about 1×10\hat{ }10 cfu's, or about 1×10\hat{ }6 cfu's to about 7.5×10\hat{ }9 cfu's, or about 1×10\hat{ }6 cfu's to about 5×10\hat{ }9 cfu's, or about 1×10\hat{ }6 cfu's to about 2.5×10\hat{ }9 cfu's, or about 1×10\hat{ }6 cfu's to about 1.25×10\hat{ }9 cfu's, or about 1×10\hat{ }6 cfu's to about 1×10\hat{ }9 cfu's, or about 1×10\hat{ }6 cfu's to about 1×10\hat{ }8 cfu's, or about 1×10\hat{ }6 cfu's to about 1×10\hat{ }7 cfu's, or about 1×10\hat{ }7 cfu's to about 1×10\hat{ }13 cfu's, or about 1×10\hat{ }7 cfu's to about 1×10\hat{ }12 cfu's, or about 1×10\hat{ }7 cfu's to about 1×10\hat{ }11 cfu's, or about 1×10\hat{ }7 cfu's to about 3×10\hat{ }10 cfu's, or about 1×10\hat{ }7 cfu's to about 1.5×10\hat{ }10 cfu's, or about 1×10\hat{ }7 cfu's to about 1×10\hat{ }10 cfu's, or about 1×10\hat{ }7 cfu's to about 7.5×10\hat{ }9 cfu's, or about 1×10\hat{ }7 cfu's to about 5×10\hat{ }9 cfu's, or about 1×10\hat{ }7 cfu's to about 2.5×10\hat{ }9 cfu's, or about 1×10\hat{ }7 cfu's to about 1.25×10\hat{ }9 cfu's, or about 1×10\hat{ }7 cfu's to about 1×10\hat{ }9 cfu's, or about 1×10\hat{ }7 cfu's to about 1×10\hat{ }8 cfu's, or about 1×10\hat{ }8 cfu's to about 1×10\hat{ }13 cfu's, or about 1×10\hat{ }8 cfu's to about 1×10\hat{ }12 cfu's, or about 1×10\hat{ }8 cfu's to about 1×10\hat{ }11 cfu's, or about 1×10\hat{ }8 cfu's to about 3×10\hat{ }10 cfu's, or about 1×10\hat{ }8 cfu's to about 1.5×10\hat{ }10 cfu's, or about 1×10\hat{ }8 cfu's to about 1×10\hat{ }10 cfu's, or about 1×10\hat{ }8 cfu's to about 7.5×10\hat{ }9 cfu's, or about 1×10\hat{ }8 cfu's to about 5×10\hat{ }9 cfu's, or about 1×10\hat{ }8 cfu's to about 2.5×10\hat{ }9 cfu's, or about 1×10\hat{ }8 cfu's to about 1.25×10\hat{ }9 cfu's, or about 1×10\hat{ }8 cfu's to about 1×10\hat{ }9 cfu's, or about 1×10\hat{ }9 cfu's to about 1×10\hat{ }13 cfu's, or about 1×10\hat{ }9 cfu's to about 1×10\hat{ }12 cfu's, or about 1×10\hat{ }9 cfu's to about 1×10\hat{ }11 cfu's, or about 1×10\hat{ }9 cfu's to about 3×10\hat{ }10 cfu's, or about 1×10\hat{ }9 cfu's to about 1.5×10\hat{ }10 cfu's, or about 1×10\hat{ }9 cfu's to about 1×10\hat{ }10 cfu's, or about 1×10\hat{ }9 cfu's to about 7.5×10\hat{ }9 cfu's, or about 1×10\hat{ }9 cfu's to about 5×10\hat{ }9 cfu's, or about 1×10\hat{ }9 cfu's to about 2.5×10\hat{ }9 cfu's, or about 1×10\hat{ }9 cfu's to about 1.25×10\hat{ }9 cfu's, or about 1.25×10\hat{ }9 cfu's to about 1×10\hat{ }13 cfu's, or about 1.25×10\hat{ }9 cfu's to about 1×10\hat{ }12 cfu's, or about 1.25×10\hat{ }9 cfu's to about 1×10\hat{ }11 cfu's, or about 1.25×10\hat{ }9 cfu's to about 3×10\hat{ }10 cfu's, or about 1.25×10\hat{ }9 cfu's to about 1.5×10\hat{ }10 cfu's, or about 1.25×10\hat{ }9 cfu's to about 1×10\hat{ }10 cfu's, or about 1.25×10\hat{ }9 cfu's to about 7.5×10\hat{ }9 cfu's, or about 1.25×10\hat{ }9 cfu's to about 5×10\hat{ }9 cfu's, or about 1.25×10\hat{ }9 cfu's to about 2.5×10\hat{ }9 cfu's, or about 2.5×10\hat{ }9 cfu's to about 1×10\hat{ }13 cfu's, or about 2.5×10\hat{ }9 cfu's to about 1×10\hat{ }12 cfu's, or about 2.5×10\hat{ }9 cfu's to about 1×10\hat{ }11 cfu's, or about 2.5×10\hat{ }9 cfu's to about 3×10\hat{ }10 cfu's, or about 2.5×10\hat{ }9 cfu's to about 1.5×10\hat{ }10 cfu's, or about 2.5×10\hat{ }9 cfu's to about 1×10\hat{ }10 cfu's, or about 2.5×10\hat{ }9 cfu's to about 7.5×10\hat{ }9 cfu's, or about 2.5×10\hat{ }9 cfu's to about 5×10\hat{ }9 cfu's, or about 5×10\hat{ }9 cfu's to about 1×10\hat{ }13 cfu's, or about 5×10\hat{ }9 cfu's to about 1×10\hat{ }12 cfu's, or about 5×10\hat{ }9 cfu's to about 1×10\hat{ }11 cfu's, or about 5×10\hat{ }9 cfu's to about 3×10\hat{ }10 cfu's, or about 5×10\hat{ }9 cfu's to about 1.5×10\hat{ }10 cfu's, or about 5×10\hat{ }9 cfu's to about 1×10\hat{ }10 cfu's, or about 5×10\hat{ }9 cfu's to about 7.5×10\hat{ }9 cfu's, or about 7.5×10\hat{ }9 cfu's to about 1×10\hat{ }13 cfu's, or about 7.5×10\hat{ }9 cfu's to about 1×10\hat{ }12 cfu's, or about 7.5×10\hat{ }9 cfu's to about 1×10\hat{ }11 cfu's, or about 7.5×10\hat{ }9 cfu's to about 3×10\hat{ }10 cfu's, or about 7.5×10\hat{ }9 cfu's to about 1.5×10\hat{ }10 cfu's, or about 7.5×10\hat{ }9 cfu's to about 1×10\hat{ }10 cfu's, or about 1×10\hat{ }10 cfu's to about 1×10\hat{ }13 cfu's, or about 1×10\hat{ }10 cfu's to about 1×10\hat{ }12 cfu's, or about 1×10\hat{ }10 cfu's to about 1×10\hat{ }11 cfu's, or about 1×10\hat{ }10 cfu's to about 3×10\hat{ }10 cfu's, or about 1×10\hat{ }10 cfu's to about 1.5×10\hat{ }10 cfu's, or about 1.5×10\hat{ }10 cfu's to about 1×10\hat{ }13 cfu's, or about 1.5×10\hat{ }10 cfu's to about 1×10\hat{ }12 cfu's, or about 1.5×10\hat{ }10 cfu's to about 1×10\hat{ }11 cfu's, or about 1.5×10\hat{ }10 cfu's to about 3×10\hat{ }10 cfu's, or about 3×10\hat{ }10 cfu's to about 1×10\hat{ }13 cfu's, or about 13×10\hat{ }10 cfu's to about 1×10\hat{ }12 cfu's, or about 3×10\hat{ }10 cfu's to about 1×10\hat{ }11 cfu's, or about 1×10\hat{ }11 cfu's to about 1×10\hat{ }13 cfu's, or about 1×10\hat{ }11 cfu's to about 1×10\hat{ }12 cfu's, or about 1×10\hat{ }12 cfu's to about 1×10\hat{ }13 cfu's. In some embodiments, one or more strains of probiotic microbes (e.g. Lactobacillus brevis, Lactobacillus plantarum, Leuconostoc mesenteroides, and/or Pichia kudriavzevii) are ingested in an amount of about 1.5×10\hat{ }10 cfu's per serving, or about 3×10\hat{ }10 cfu's per serving, or about 1.25×10\hat{ }9 cfu's per serving, or about 2.5×10\hat{ }9 cfu's per serving, or about 5×10\hat{ }9 cfu's per serving, or about 7.5×10\hat{ }9 cfu's per serving. In some embodiments, one or more strains of probiotic microbes (e.g. Lactobacillus brevis, Lactobacillus plantarum, Leuconostoc mesenteroides, and/or Pichia kudriavzevii) are ingested in an amount of about 1.5×10\hat{ }10 cfu's per day, or about 3×10\hat{ }10 cfu's per day, or about 1.25×10\hat{ }9 cfu's per day, or about 2.5×10\hat{ }9 cfu's per day, or about 5×10\hat{ }9 cfu's per day, or about 7.5×10\hat{ }9 cfu's per day.

In another embodiment, one or more strains of probiotic microbes are administered as part of a dairy product. In an embodiment, a typical serving size for a dairy product such as fluid milk is 240 g. In other embodiments, a serving size is 245 g, or 240 g to 245 g, or 227 to 300 g. In an embodiment the dairy product is yogurt. Yogurt can have a serving size of 4 oz, or 6 oz, or 8 oz, or 4 oz to 10 oz, or half cup, or 1 cup, or 113 g, or 170 g, or 227 g, or 245 g or 277 g, or 100 g to 350 g.

In an embodiment, probiotic microbes are given as live cultured microbes, e.g., in combination with a prebiotic (e.g., comprising or consisting essentially of dried fruit or vegetable powder, FOS, GOS, and/or other appropriate polysaccharide) and, optionally, other substances. The dose can be 0.001 mg to 1 mg, or 0.5 mg to 5 mg, or 1 mg to 1000 mg, or 2 mg to 200 mg, or 2 mg to 100 mg, or 2 mg to 50 mg, or 2 mg to 25 mg, or 2 mg to 20 mg, or 4 mg to 50 mg, or 4 mg to 25 mg, or 5 mg to 50 mg, or 5 mg to 40 mg, or 5 mg to 30 mg or 5 mg to 25 mg, or 5 mg to 20 mg, or 10 mg to 100 mg, or 10 mg to 75 mg, or 10 mg to 50 mg, or 10 mg to 25 mg, or 10 mg to 15 mg, or 20 mg to 100 mg, or 20 mg to 75 mg, or 20 mg to 50 mg, or 20 mg to 40 mg, or 20 mg to 30 mg, or 20 mg to 25 mg, or 50 mg to 200 mg, or 50 mg to 150 mg, or 50 mg to 100 mg, or 50 mg to 75 mg, or 100 mg to 1000 mg, or 100 mg to 800 mg, or 100 mg to 600 mg, or 100 mg to 500 mg, or 100 mg to 400 mg, or 100 mg to 300 mg, or 100 mg to 200 mg, or 100 mg to 150 mg, or 200 mg to 1000 mg, or about 10, about 11, about 12, about 12.5, about 13, about 14, about 15, about 20, about 25, about 30, about 35, about 40, about 45, about 50, about 60, about 70, about 80, about 90, about 100, about 125, about 150, about 175, or about 200 mg of probiotic microbial cell culture dry weight per serving or per daily dose. In certain embodiments, Lactobacillus brevis is used in a dose of about 25 mg to about 50 mg per serving or per day. In certain embodiments, Lactobacillus plantarum is used in a dose of about 25 mg to about 50 mg per serving or per day. In certain embodiments, Leuconostoc mesenteroides is used in a dose of about 25 mg to about 50 mg per serving or per day. In certain embodiments, Pichia kudriavzevii is used in a dose of about 100 mg to about 200 mg per serving or per day. In certain embodiments, L. acidophilus is used in a dose of 12.5 mg per serving or per day. In some embodiments, as the administration of a prebiotic (e.g., comprising or consisting essentially of dried fruit or vegetable powder, FOS, GOS, and/or other appropriate polysaccharide) dose to a subject increases, the dose of microbes increases as well. For example, an initial dose of a prebiotic (e.g., comprising or consisting essentially of dried fruit or vegetable powder, FOS, GOS, and/or other appropriate polysaccharides) can be 0.3 g to 0.4 g, e.g., 0.35 g, given in combination with 25-50 mg, e.g., 37.5 mg, of a lactic acid bacterium. The dose of a prebiotic (e.g., comprising or consisting essentially of dried fruit or vegetable powder, FOS, GOS, and/or other appropriate polysaccharide) can be increased incrementally by 0.3 g to 0.4 g, e.g., 0.35 g, and the accompanying dose of the lactic acid bacterium can be increased by 25-50 mg, e.g., 37.5 mg, of the bacterium. Similarly, proportional incremental decreases of prebiotic and probiotic doses are also contemplated.

In certain embodiments, probiotic microbes and a prebiotic (e.g., comprising or consisting essentially of dried fruit or vegetable powder, FOS, GOS, and/or other appropriate polysaccharide) are administered in the same composition. In certain embodiments, the probiotic microbes constitute 1% to 99% of the weight of the composition. For example, in certain embodiments, the probiotic microbes constitute 1% to 99%, 1% to 95%, 1% to 90%, 1% to 80%, 1% to 70%, 1% to 60%, 1% to 50%, 1% to 40%, 1% to 30%, 1% to 25%, 1% to 20%, 1% to 15%, 1% to 10%, 1% to 5%, 5% to 99%, 5% to 95%, 5% to 90%, 5% to 80%, 5% to 70%, 5% to 60%, 5% to 50%, 5% to 40%, 5% to 30%, 5% to 25%, 5% to 20%, 5% to 15%, 5% to 10%, 10% to 99%, 10% to 95%, 10% to 90%, 10% to 80%, 10% to 70%, 10% to 60%, 10% to 50%, 10% to 40%, 10% to 30%, 10% to 25%, 10% to 20%, 10% to 15%, 15% to 99%, 15% to 95%, 15% to 90%, 15% to 80%, 15% to 70%, 15% to 60%, 15% to 50%, 15% to 40%, 15% to 30%, 15% to 25%, 15% to 20%, 20% to 99%, 20% to 95%, 20% to 90%, 20% to 80%, 20% to 70%, 20% to 60%, 20% to 50%, 20% to 40%, 20% to 30%, 20% to 25%, 25% to 99%, 25% to 95%, 25% to 90%, 25% to 80%, 25% to 70%, 25% to 60%, 25% to 50%, 25% to 40%, 25% to 30%, 30% to 99%, 30% to 95%, 30% to 90%, 30% to 80%, 30% to 70%, 30% to 60%, 30% to 50%, 30% to 40%, 40% to 99%, 40% to 95%, 40% to 90%, 40% to 80%, 40% to 70%, 40% to 60%, 40% to 50%, 50% to 99%, 50% to 95%, 50% to 90%, 50% to 80%, 50% to 70%, 50% to 60%, 60% to 99%, 60% to 95%, 60% to 90%, 60% to 80%, 60% to 70%, 70% to 99%, 70% to 95%, 70% to 90%, 70% to 80%, 80% to 99%, 80% to 95%, 80% to 90%, 90% to 99%, 90% to 95%, or 95% to 99% of the weight of the composition. In certain embodiments, the prebiotic constitutes 1% to 99% of the weight of the composition. For example, in certain embodiments, the prebiotic constitutes 1% to 99%, 1% to 95%, 1% to 90%, 1% to 80%, 1% to 70%, 1% to 60%, 1% to 50%, 1% to 40%, 1% to 30%, 1% to 25%, 1% to 20%, 1% to 15%, 1% to 10%, 1% to 5%, 5% to 99%, 5% to 95%, 5% to 90%, 5% to 80%, 5% to 70%, 5% to 60%, 5% to 50%, 5% to 40%, 5% to 30%, 5% to 25%, 5% to 20%, 5% to 15%, 5% to 10%, 10% to 99%, 10% to 95%, 10% to 90%, 10% to 80%, 10% to 70%, 10% to 60%, 10% to 50%, 10% to 40%, 10% to 30%, 10% to 25%, 10% to 20%, 10% to 15%, 15% to 99%, 15% to 95%, 15% to 90%, 15% to 80%, 15% to 70%, 15% to 60%, 15% to 50%, 15% to 40%, 15% to 30%, 15% to 25%, 15% to 20%, 20% to 99%, 20% to 95%, 20% to 90%, 20% to 80%, 20% to 70%, 20% to 60%, 20% to 50%, 20% to 40%, 20% to 30%, 20% to 25%, 25% to 99%, 25% to 95%, 25% to 90%, 25% to 80%, 25% to 70%, 25% to 60%, 25% to 50%, 25% to 40%, 25% to 30%, 30% to 99%, 30% to 95%, 30% to 90%, 30% to 80%, 30% to 70%, 30% to 60%, 30% to 50%, 30% to 40%, 40% to 99%, 40% to 95%, 40% to 90%, 40% to 80%, 40% to 70%, 40% to 60%, 40% to 50%, 50% to 99%, 50% to 95%, 50% to 90%, 50% to 80%, 50% to 70%, 50% to 60%, 60% to 99%, 60% to 95%, 60% to 90%, 60% to 80%, 60% to 70%, 70% to 99%, 70% to 95%, 70% to 90%, 70% to 80%, 80% to 99%, 80% to 95%, 80% to 90%, 90% to 99%, 90% to 95%, or 95% to 99% of the weight of the composition.

### FOS, GOS, or Other Appropriate Polysaccharide Formulations

A. Formulations introduction

In an aspect, a prebiotic composition for the treatment of one or more musculoskeletal disorder is provided. In an aspect, a prebiotic composition for the treatment of inflammation is provided. In an aspect, a prebiotic composition for the management of one or more symptoms of perimenopause, menopause, or postmenopause is provided. In an embodiment, a prebiotic composition comprises inulin, FOS, lactulose, GOS, raffinose, stachyose, or a combination thereof. In addition, other plant-derived polysaccharides such as xylan, pectin, isomalto-oligosaccharides, gentio-oligosaccharides, 4-O-methyl glucuronoxylan (GX), neutral arabinoxylan (AX), heteroxylan (HX) can be combined with the probiotics to enhance bacterial metabolic function. Some of these can be derived from plant material found in the plant host from which the probiotics were isolated from. Therefore, the probiotics are adapted to assimilate and digest the rich complexity and variety of polysaccharides present in the plant that play a role during digestion by the consumption of an animal.

In some embodiments, a prebiotic composition comprises or consists of FOS, GOS, or other appropriate polysaccharide. In an, embodiment a prebiotic composition comprises FOS, GOS, other, and one or more digestible saccharides. Digestible saccharides are saccharides that are digestible by humans and include, but are not limited to lactose, glucose, and galactose. In an embodiment, a prebiotic composition comprises FOS, GOS, or other appropriate polysaccharide, and less than 20% weight/weight of one or more digestible saccharides (e.g. lactose, glucose, or galactose). In an embodiment, a prebiotic composition comprises FOS, GOS, or other appropriate polysaccharide, and less than 10% of one or more digestible saccharides. In an embodiment, a prebiotic composition comprises FOS, GOS, or other appropriate polysaccharide, and less than 5% of one or more digestible saccharides. In an embodiment, a prebiotic composition contains less than 5% lactose. In an embodiment, a prebiotic composition contains less than 4% lactose. In an embodiment, a prebiotic composition contains less than 3% lactose. In an embodiment, a prebiotic composition contains less than 2% lactose. In an embodiment, a prebiotic composition contains less than 1% lactose. In an embodiment, a prebiotic composition contains less than 0.5% lactose. In an embodiment, a prebiotic composition contains less than 0.4% lactose. In an embodiment, a prebiotic composition contains less than 0.3% lactose. In an embodiment, a prebiotic composition contains less than 0.2% lactose. In an embodiment, a prebiotic composition contains less than 0.1% lactose. In an embodiment, a prebiotic composition contains less than 0.05% lactose. In an embodiment, a prebiotic composition contains less than 0.01% lactose. In an embodiment, a prebiotic composition contains less than 0.005% lactose. In an embodiment, a prebiotic composition comprises FOS, GOS, or other appropriate polysaccharide, and essentially no lactose. In an embodiment, a prebiotic composition does not contain any lactose. In an embodiment, a prebiotic composition contains FOS, GOS, or other appropriate polysaccharide, and at least one probiotic bacteria strain. In an embodiment, a prebiotic composition comprises FOS, GOS, or other appropriate polysaccharide, and optionally one or more of lactose, at least one probiotic bacteria strain, and a buffer. Additional ingredients include ingredients to improve handling, preservatives, antioxidants, flavorings and the like.

In certain embodiments, a prebiotic composition comprises FOS, GOS, or other appropriate polysaccharide, or a probiotic. In some embodiments, a prebiotic composition is in the form of a powder, tablet, capsule, or liquid. In some embodiments, a prebiotic composition can be administered with a dairy product and is in the form of milk or other common dairy product such as a yogurt, shake, smoothie, cheese, and the like.

In embodiments where a prebiotic composition comprises less than 100% by weight of FOS, GOS, or other appropriate polysaccharide, the remaining ingredients can be any suitable ingredients intended for the consumption of the subject in need thereof, e.g., human, including, but not limited to, other prebiotics (e.g., FOS), a buffer, one or more digestible saccharides (e.g. lactose, glucose, or galactose), ingredients intended to inhibit clumping and increase pourability, such as silicone dioxide, magnesium stearate, microcrystalline cellulose, or similar ingredients as are well-known in the art. Remaining ingredients can also include ingredients to improve handling, preservatives, antioxidants, flavorings, and the like.

### B. Buffer Components

One or more buffers, optionally with a calcium counter ion, can also be administered in methods and compositions described herein. Any buffer suitable for consumption by the subject being treated, e.g., human, are useful for the compositions herein. The buffer neutralizes stomach acidity, which can, e.g., allow live microbes to reach the gut. Buffers include citrates, phosphates, and the like. One embodiment utilizes a buffer with a calcium counter ion, such as Calcium Phosphate Tribasic. The calcium can serve to restore the calcium that many lactose intolerant subjects are missing in their diet. Calcium phosphate can protect Lactobacillus acidophilus from bile. Calcium phosphate can help neutralize stomach acidity.

In an embodiment, a buffer such as calcium phosphate is given prior to beginning treatment with a prebiotic composition (such as a composition comprising or consisting essentially of dried fruit or vegetable powder, FOS, GOS, and/or other appropriate polysaccharide), optionally in conjunction with administration of microbes. As used herein FOS indications one or more fructo-oligosaccharides and GOS indicates one or more galacto-oligosaccharides. In an embodiment, a buffer such as calcium phosphate is given in conjunction with treatment with a prebiotic composition (e.g., a composition comprising or consisting essentially of dried fruit or vegetable powder, FOS, GOS, and/or other appropriate polysaccharide), for part or all of the treatment with lactose. Thus, in an embodiment, some or all doses of a prebiotic composition are accompanied by a dose of a buffer such as calcium phosphate. In an embodiment, a buffer such as calcium phosphate is given initially with a prebiotic composition (such as a composition comprising or consisting essentially of dried fruit or vegetable powder, FOS, GOS, and/or other appropriate polysaccharide), but then its use is discontinued. For example, the initial one, two, three, four, five, six, seven, eight, nine, ten, or more than ten days of treatment with a prebiotic composition can include doses of a buffer such as calcium phosphate, with the use of the buffer discontinued after that time. In an embodiment, a buffer such as calcium phosphate can be given for the first two days of treatment, and then the administration of buffer is discontinued. In an embodiment, a buffer such as calcium phosphate, either alone or in combination with other substances or treatments is used after the treatment with a prebiotic composition is terminated. A buffer such as calcium phosphate can be taken for any suitable period after the termination of treatment with lactose, and can be taken daily or at regular or irregular intervals. Doses can be as described below.

Numerous buffers suitable for human consumption are known in the art, and any suitable buffer can be used in the methods and compositions described herein. Calcium triphosphate is an exemplary buffer, and its counterion supplies a nutrient that is often lacking in lactose-intolerant subjects, i.e. calcium. In an embodiment, a buffer can be used in a dose from 2 mg to 2000 mg, or 4 mg to 400 mg, or 4 mg to 200 mg, or 4 mg to 100 mg, or 8 mg to 50 mg, or 10 mg to 40 mg, or 20 mg to 30 mg, or 20, 21, 22, 23, 24, 25, 26, 27, 28, 29 or 30 mg. In an embodiment, a prebiotic composition further comprises an amount of a buffer from 1-50 mg, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg. In an embodiment, a buffer is used in a dose of 25 mg. In an embodiment, calcium phosphate is used in a dose of 25 mg. The dose can be given in combination with a prebiotic composition (e.g., a composition comprising or consisting essentially of dried fruit or vegetable powder, FOS, GOS, and/or other appropriate polysaccharide). In an embodiment, as a prebiotic composition dose increases, the dose of buffer increases as well. For example, an initial dose of a prebiotic composition can be 0.6 g to 1.0 g, e.g., 0.8 g, given in combination with 20-30 mg, e.g., 25 mg, of buffer, e.g., calcium phosphate. The dose of a prebiotic composition can be increased incrementally by 0.6 g to 1.0 g, e.g., 0.8 g, and the accompanying dose of buffer, e.g., calcium phosphate, can be increased by 20-30 mg, e.g., 25 mg, of buffer, e.g., calcium phosphate.

### C. Compositions Comprising GOS and at Least One Probiotic Microbial Strain

In an embodiment, a prebiotic composition comprises FOS, GOS, or other appropriate polysaccharide, and at least one probiotic microbial strain (e.g. probiotic bacteria and/or fungi). The FOS, GOS, or other appropriate polysaccharide can comprise more than 1% of the weight of the composition while the at least one probiotic microbial strain will typically comprise less than 10%, 5%, 4%, 3%, or 2% by weight of the compositions. For example, the FOS, GOS, or other appropriate polysaccharide can be present at 1-99.75% by weight and the at least one probiotic bacteria strain at 0.25-2% by weight, or the FOS, GOS, or other appropriate polysaccharide can be present at 89-96% by weight and the bacteria at 1.2-3.7% by weight. In an embodiment, FOS, GOS, or other appropriate polysaccharide are present at 92% by weight and at least one probiotic microbial strain, (e.g., Lactobacillus brevis, Lactobacillus plantarum, Leuconostoc mesenteroides, Pichia kudriavzevii, and/or other microbes from Table 1 or Table 2), is present at 1.5% by weight. In an embodiment, FOS, GOS, or other appropriate polysaccharide are present at 2% by weight and at least one probiotic microbial strain, (e.g., Lactobacillus brevis, Lactobacillus plantarum, Leuconostoc mesenteroides, Pichia kudriavzevii, and/or other microbes from Table 1 or Table 2), is present at 1.5% by weight. In another embodiment, FOS, GOS, or other appropriate polysaccharide are present at 93% by weight and at least one probiotic microbial strain, (e.g., Lactobacillus brevis, Lactobacillus plantarum, Leuconostoc mesenteroides, Pichia kudriavzevii, and/or other microbes from Table 1 or Table 2), is present at 1.5% by weight. In another embodiment, FOS, GOS, or other appropriate polysaccharide are present at 94% by weight and at least one probiotic microbial strain, (e.g., Lactobacillus brevis, Lactobacillus plantarum, Leuconostoc mesenteroides, Pichia kudriavzevii, and/or other microbes from Table 1 or Table 2), is present at 1.5% by weight. In another embodiment, FOS, GOS, or other appropriate polysaccharide are present at 95% by weight and at least one probiotic microbial strain, (e.g., Lactobacillus brevis, Lactobacillus plantarum, Leuconostoc mesenteroides, Pichia kudriavzevii, and/or or other microbes from Table 1 or Table 2), is present at 1.5% by weight. In another embodiment, FOS, GOS, or other appropriate polysaccharide are present at 96% by weight and at least one probiotic microbial strain, (e.g. Lactobacillus brevis, Lactobacillus plantarum, Leuconostoc mesenteroides, Pichia kudriavzevii, and/or other microbes from Table 1 or Table 2), is present at 1.5% by weight. In another embodiment, FOS, GOS, or other appropriate polysaccharide are present at 97% by weight and at least one probiotic microbial strain, (e.g., Lactobacillus brevis, Lactobacillus plantarum, Leuconostoc mesenteroides, Pichia kudriavzevii, and/or other microbes from Table 1 or Table 2), is present at 1.5% by weight. In another embodiment, FOS, GOS, or other appropriate polysaccharide are present at 98% by weight and at least one probiotic microbial strain, (e.g., Lactobacillus brevis, Lactobacillus plantarum, Leuconostoc mesenteroides, Pichia kudriavzevii, and/or other microbes from Table 1 or Table 2), is present at 1.5% by weight. In another embodiment, FOS, GOS, or other appropriate polysaccharide are present at 98.5% by weight and at least one probiotic microbial strain, (e.g., Lactobacillus brevis, Lactobacillus plantarum, Leuconostoc mesenteroides, Pichia kudriavzevii, and/or other microbes from Table 1 or Table 2), is present at 1.5% by weight. If the at least one probiotic microbial strain and FOS, GOS, or other appropriate polysaccharide do not make up 100% by weight of the prebiotic composition, the remaining ingredients can be any suitable ingredients intended for consumption by the subject in need thereof, e.g., human, including, but not limited to, other prebiotics (e.g., FOS and/or dried fruit or vegetable powder), one or more buffers, digestible saccharides (e.g. lactose, glucose, or galactose), ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### D. Compositions Comprising FOS, GOS, or Other Appropriate Polysaccharide and a Buffer

In another embodiment, a prebiotic composition comprises FOS, GOS, or other appropriate polysaccharide and a buffer (e.g., calcium phosphate tribasic). For example, FOS, GOS, or other appropriate polysaccharide can be present at 1-100% by weight and the buffer at 0.50-4% by weight, or FOS, GOS, or other appropriate polysaccharide can be present at 1-96% by weight and the buffer at 1 to 3.75% by weight. In an embodiment, FOS, GOS, or other appropriate polysaccharide are present at 1% by weight and buffer is present at 3% by weight. In an embodiment, FOS, GOS, or other appropriate polysaccharide are present at 5% by weight and buffer is present at 3% by weight. In an embodiment, FOS, GOS, or other appropriate polysaccharide are present at 10% by weight and buffer is present at 3% by weight. In an embodiment, FOS, GOS, or other appropriate polysaccharide are present at 15% by weight and buffer is present at 15% by weight. In an embodiment, FOS, GOS, or other appropriate polysaccharide are present at 20% by weight and buffer is present at 3% by weight. In an embodiment, FOS, GOS, or other appropriate polysaccharide are present at 25% by weight and buffer is present at 3% by weight. In an embodiment, FOS, GOS, or other appropriate polysaccharide are present at 30% by weight and buffer is present at 3% by weight. In an embodiment, FOS, GOS, or other appropriate polysaccharide are present at 35% by weight and buffer is present at 3% by weight. In an embodiment, FOS, GOS, or other appropriate polysaccharide are present at 40% by weight and buffer is present at 3% by weight. In an embodiment, FOS, GOS, or other appropriate polysaccharide are present at 50% by weight and buffer is present at 3% by weight. In an embodiment, FOS, GOS, or other appropriate polysaccharide are present at 60% by weight and buffer is present at 3% by weight. In an embodiment, FOS, GOS, or other appropriate polysaccharide are present at 70% by weight and buffer is present at 3% by weight. In another embodiment, FOS, GOS, or other appropriate polysaccharide are present at 90% by weight and buffer is present at 3% by weight. In another embodiment, FOS, GOS, or other appropriate polysaccharide are present at 92% by weight and buffer is present at 3% by weight. In another embodiment, FOS, GOS, or other appropriate polysaccharide are present at 93% by weight and buffer is present at 3% by weight. In another embodiment, FOS, GOS, or other appropriate polysaccharide are present at 94% by weight and buffer is present at 3% by weight. In another embodiment, FOS, GOS, or other appropriate polysaccharide are present at 95% by weight and buffer is present at 3% by weight. In another embodiment, FOS, GOS, or other appropriate polysaccharide are present at 96% by weight and buffer is present at 3% by weight. In another embodiment, FOS, GOS, or other appropriate polysaccharide are present at 97% by weight and buffer is present at 2% by weight. In another embodiment, FOS, GOS, or other appropriate polysaccharide are present at 98% by weight and buffer is present at 1% by weight. In another embodiment, FOS, GOS, or other appropriate polysaccharide are present at 99% by weight and buffer is present at 1% by weight. In another embodiment, FOS, GOS, or other appropriate polysaccharide are present at 100% by weight and buffer is present at less than 1% by weight. If the buffer and FOS, GOS, or other appropriate polysaccharide do not make up 100% by weight of the composition, the remaining ingredients can be any suitable ingredients intended for consumption by the subject (e.g., a human) including, but not limited to, probiotics (e.g., beneficial microbes, bacteria and/or fungi) or other prebiotics (e.g., FOS and/or dried fruit or vegetable powder), but also including ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### E. Compositions Comprising a Digestible Saccharide, a Probiotic Bacteria, and FOS, GOS, or Other Appropriate Polysaccharide

In an embodiment, a prebiotic composition comprises a digestible saccharide (e.g. lactose, glucose, or galactose), at least one probiotic microbe (e.g. bacteria and/or yeast, e.g., Lactobacillus brevis, Lactobacillus plantarum, Leuconostoc mesenteroides, Pichia kudriavzevii, and/or other microbes from Table 1 and/or Table 2), and FOS, GOS, or other appropriate polysaccharide. In an embodiment, lactose can be present at 1-20% by weight, microbes at 0.25-20.10% by weight, and FOS, GOS, or other appropriate polysaccharide at 1-98.75% by weight. In another embodiment lactose can be present at 5-20% by weight, microbes at 0.91-1.95% by weight, and FOS, GOS, or other appropriate polysaccharide at 1 to 96% by weight. In another embodiment, lactose is present at 20% by weight, microbes at 1.5% by weight, and FOS, GOS, or other appropriate polysaccharide are present at 1% by weight. In another embodiment, lactose is present at 20% by weight, microbes at 1.5% by weight, and FOS, GOS, or other appropriate polysaccharide are present at 50% by weight. In another embodiment, lactose is present at 20% by weight, microbes at 1.5% by weight, and FOS, GOS, or other appropriate polysaccharide are present at 60% by weight. In another embodiment, lactose is present at 20% by weight, microbes at 1.5% by weight, and FOS, GOS, or other appropriate polysaccharide are present at 70% by weight. In another embodiment, lactose is present at 5% by weight, microbes at 1.5% by weight, and FOS, GOS, or other appropriate polysaccharide are present at 90% by weight. In another embodiment, lactose is present at 5% by weight, microbes at 1.5% by weight, and FOS, GOS, or other appropriate polysaccharide are present at 92% by weight. In another embodiment, lactose is present at 5% by weight, microbes at 1.5% by weight, and FOS, GOS, or other appropriate polysaccharide are present at 93% by weight. In another embodiment, lactose is present at 5% by weight, microbes at 1% by weight, and FOS, GOS, or other appropriate polysaccharide are present at 94% by weight. In another embodiment, lactose is present at 4.5% by weight, microbes at 1.5% by weight, and FOS, GOS, or other appropriate polysaccharide are present at 94% by weight. In another embodiment, lactose is present at 4.5% by weight, microbes at 0.5% by weight, and FOS, GOS, or other appropriate polysaccharide are present at 95% by weight. In another embodiment, lactose is present at 3.5% by weight, microbes at 0.5% by weight, and FOS, GOS, or other appropriate polysaccharide are present at 96% by weight. In another embodiment, lactose is present at 2.5% by weight, microbes at 0.5% by weight, and FOS, GOS, or other appropriate polysaccharides are present at 97% by weight. In another embodiment, lactose is present at 1.5% by weight, microbes at 0.5% by weight, and FOS, GOS, or other appropriate polysaccharide are present at 98% by weight. In another embodiment, lactose is present at 0.5% by weight, microbes at 0.5% by weight, and FOS, GOS, or other appropriate polysaccharide are present at 99% by weight. If the microbes, FOS, GOS, or other appropriate polysaccharide and lactose do not make up 100% of the composition, the remaining ingredients can be any suitable ingredients intended for consumption by the subject, e.g., a human, including, but not limited to a buffer, digestible saccharides (e.g., lactose, glucose, or galactose), ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### F. Compositions Comprising FOS, GOS, or Other Appropriate Polysaccharide, a Probiotic Bacteria, and Buffer

In an embodiment, a prebiotic composition comprises FOS, GOS, or other appropriate polysaccharide, at least one probiotic microbial strain (e.g. a bacterial and/or fungal strain, e.g., Lactobacillus brevis, Lactobacillus plantarum, Leuconostoc mesenteroides, Pichia kudriavzevii, and/or another microbe of Table 1 or Table 2), and buffer. In an embodiment, FOS, GOS, or other appropriate polysaccharide can be present at 1-100% by weight, a probiotic microbial strain at 0.25-2% by weight, and the buffer at 0.50-4% by weight. In another embodiment, FOS, GOS, or other appropriate polysaccharide can be present at 1-95% by weight, a probiotic b microbial strain at 0.91-1.95% by weight, and the buffer at 1.2-30.75% by weight. In another embodiment, FOS, GOS, or other appropriate polysaccharide are present at 1% by weight, a probiotic microbial strain at 1.5% by weight, and buffer is present at 3% by weight. In another embodiment, FOS, GOS, or other appropriate polysaccharide are present at 5% by weight, a probiotic microbial strain at 1.5% by weight, and buffer is present at 3% by weight. In another embodiment, FOS, GOS, or other appropriate polysaccharide are present at 10% by weight, a probiotic microbial strain at 1.5% by weight, and buffer is present at 3% by weight. In another embodiment, FOS, GOS, or other appropriate polysaccharide are present at 15% by weight, a probiotic microbial strain at 1.5% by weight, and buffer is present at 3% by weight. In another embodiment, FOS, GOS, or other appropriate polysaccharide are present at 20% by weight, a probiotic microbial strain at 1.5% by weight, and buffer is present at 3% by weight. In another embodiment, FOS, GOS, or other appropriate polysaccharide are present at 25% by weight, a probiotic microbial strain at 1.5% by weight, and buffer is present at 3% by weight. In another embodiment, FOS, GOS, or other appropriate polysaccharide are present at 30% by weight, a probiotic microbial strain at 1.5% by weight, and buffer is present at 3% by weight. In another embodiment, FOS, GOS, or other appropriate polysaccharide are present at 35% by weight, a probiotic microbial strain at 1.5% by weight, and buffer is present at 3% by weight. In another embodiment, FOS, GOS, or other appropriate polysaccharide are present at 40% by weight, a probiotic microbial strain at 1.5% by weight, and buffer is present at 3% by weight. In another embodiment, FOS, GOS, or other appropriate polysaccharide are present at 50% by weight, a probiotic microbial strain at 1.5% by weight, and buffer is present at 3% by weight. In another embodiment, FOS, GOS, or other appropriate polysaccharide are present at 60% by weight, a probiotic microbial strain at 1.5% by weight, and buffer is present at 3% by weight. In another embodiment, FOS, GOS, or other appropriate polysaccharide are present at 70% by weight, a probiotic microbial strain at 1.5% by weight, and buffer is present at 3% by weight. In another embodiment, FOS, GOS, or other appropriate polysaccharide are present at 90% by weight, a probiotic microbial strain at 1.5% by weight, and buffer is present at 3% by weight. In another embodiment, FOS, GOS, or other appropriate polysaccharide are present at 92% by weight, a probiotic microbial strain at 1.5% by weight, and buffer is present at 3% by weight. In another embodiment, FOS, GOS, or other appropriate polysaccharide are present at 93% by weight, a probiotic microbial strain at 1.5% by weight, and buffer is present at 3% by weight. In another embodiment, FOS, GOS, or other appropriate polysaccharide are present at 94% by weight, a probiotic microbial strain at 1.5% by weight, and buffer is present at 3% by weight. In another embodiment, FOS, GOS, or other appropriate polysaccharide are present at 95% by weight, a probiotic microbial strain at 1.5% by weight, and buffer is present at 3% by weight. In another embodiment, FOS, GOS, or other appropriate polysaccharide are present at 96% by weight, a probiotic microbial strain at 1.5% by weight, and buffer is present at 2% by weight. In another embodiment, FOS, GOS, or other appropriate polysaccharide are present at 97% by weight, a probiotic microbial strain at 1.5% by weight, and buffer is present at 1.5% by weight. In another embodiment, FOS, GOS, or other appropriate polysaccharide are present at 99% by weight, a probiotic microbial strain at 0.5% by weight, and buffer is present at 0.5% by weight. In another embodiment, FOS, GOS, or other appropriate polysaccharide are present at 100% by weight, a probiotic microbial strain at less than 0.5% by weight, and buffer is present at less than 0.5% by weight. If the probiotic microbial strain, buffer, and FOS, GOS, or other appropriate polysaccharide do not make up 100% of the composition, the remaining ingredients can be any suitable ingredients intended for the consumption of a subject (e.g., human) including, but not limited to, other prebiotics (e.g., FOS or dried fruit or vegetable powder), digestible saccharides (e.g., lactose, glucose or galactose), ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### G. Compositions Comprising a Digestible Saccharide, FOS, GOS, or Other Appropriate Polysaccharide, and a Buffer

In an embodiment, a prebiotic composition comprises a digestible saccharide (e.g. lactose, glucose, or galactose), FOS, GOS, or other appropriate polysaccharide, and a buffer. For example, lactose can be present at 1-20% by weight, FOS, GOS, or other appropriate polysaccharide at 1-100% by weight, and the buffer at 0.50-4% by weight, or the lactose can be present at 5-20% by weight, FOS, GOS, or other appropriate polysaccharide at 1-96% by weight, and the buffer at 1.2-30.75% by weight. In an embodiment, lactose is present at 20% by weight, FOS, GOS, or other appropriate polysaccharide at 1% by weight, and buffer is present at 3% by weight. In an embodiment, lactose is present at 5% by weight, FOS, GOS, or other appropriate polysaccharide at 1% by weight, and buffer is present at 3% by weight. In an embodiment, lactose is present at 20% by weight, FOS, GOS, or other appropriate polysaccharide at 10% by weight, and buffer is present at 3% by weight. In an embodiment, lactose is present at 20% by weight, FOS, GOS, or other appropriate polysaccharide at 15% by weight, and buffer is present at 3% by weight. In an embodiment, lactose is present at 20% by weight, FOS, GOS, or other appropriate polysaccharide at 20% by weight, and buffer is present at 3% by weight. In an embodiment, lactose is present at 20% by weight, FOS, GOS, or other appropriate polysaccharide at 25% by weight, and buffer is present at 3% by weight. In an embodiment, lactose is present at 20% by weight, FOS, GOS, or other appropriate polysaccharide at 30% by weight, and buffer is present at 3% by weight. In an embodiment, lactose is present at 20% by weight, FOS, GOS, or other appropriate polysaccharide at 35% by weight, and buffer is present at 3% by weight. In an embodiment, lactose is present at 20% by weight, FOS, GOS, or other appropriate polysaccharide at 40% by weight, and buffer is present at 3% by weight. In an embodiment, lactose is present at 20% by weight, FOS, GOS, or other appropriate polysaccharide at 50% by weight, and buffer is present at 3% by weight. In an embodiment, lactose is present at 20% by weight, FOS, GOS, or other appropriate polysaccharide at 60% by weight, and buffer is present at 3% by weight. In an embodiment, lactose is present at 20% by weight, FOS, GOS, or other appropriate polysaccharide at 70% by weight, and buffer is present at 3% by weight. In another embodiment, lactose is present at 5% by weight, FOS, GOS, or other appropriate polysaccharide at 90% by weight, and buffer is present at 3% by weight. In another embodiment, lactose is present at 5% by weight, FOS, GOS, or other appropriate polysaccharide at 92% by weight, and buffer is present at 3% by weight. In another embodiment, lactose is present at 4% by weight, FOS, GOS, or other appropriate polysaccharide at 93% by weight, and buffer is present at 3% by weight. In another embodiment, lactose is present at 3% by weight, FOS, GOS, or other appropriate polysaccharide at 94% by weight, and buffer is present at 3% by weight. In another embodiment, lactose is present at 2% by weight, FOS, GOS, or other appropriate polysaccharide at 95% by weight, and buffer is present at 3% by weight. In another embodiment, lactose is present at 1% by weight, FOS, GOS, or other appropriate polysaccharide at 96% by weight, and buffer is present at 3% by weight. If a suitable prebiotic, buffer and lactose do not make up 100% of the composition by weight, the remaining ingredients can be any suitable ingredients intended for consumption by a subject (e.g., human) including, but not limited to, microbes (e.g. bacteria), ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

H. Compositions Comprising a Digestible Saccharide, Microbes (e.g. Bacteria), GOS, and a Buffer

In an embodiment, a composition comprises a digestible saccharide (e.g. lactose, glucose, or galactose), microbes (e.g. bacteria and/or fungi, e.g., Lactobacillus brevis, Lactobacillus plantarum, Leuconostoc mesenteroides, Pichia kudriavzevii, and/or another microbe of Table 1 or Table 2), FOS, GOS, or other appropriate polysaccharide, and buffer. For example, lactose can be present at 1-20% by weight, microbes at 0.25-2.10% by weight, FOS, GOS, or other appropriate polysaccharide at 1-100% by weight, and the buffer at 0.50-4% by weight, or the lactose can be present at 5-20% by weight, microbes at 0.91-1.95% by weight, FOS, GOS, or other appropriate polysaccharide at 70-95% by weight, and the buffer at 1.2-30.75% by weight. In an embodiment, lactose is present at 20% by weight, microbes at 1.47% by weight, FOS, GOS, or other appropriate polysaccharide at 1% by weight, and buffer is present at 3% by weight. In an embodiment, lactose is present at 20% by weight, microbes at 1.47% by weight, FOS, GOS, or other appropriate polysaccharide at 10% by weight, and buffer is present at 3% by weight. In an embodiment, lactose is present at 20% by weight, microbes at 1.47% by weight, FOS, GOS, or other appropriate polysaccharide at 15% by weight, and buffer is present at 3% by weight. In an embodiment, lactose is present at 20% by weight, microbes at 1.47% by weight, FOS, GOS, or other appropriate polysaccharide at 20% by weight, and buffer is present at 3% by weight. In an embodiment, lactose is present at 20% by weight, microbes at 1.47% by weight, FOS, GOS, or other appropriate polysaccharide at 25% by weight, and buffer is present at 3% by weight. In an embodiment, lactose is present at 20% by weight, microbes at 1.47% by weight, FOS, GOS, or other appropriate polysaccharide at 30% by weight, and buffer is present at 3% by weight. In an embodiment, lactose is present at 20% by weight, bacteria at 1.47% by weight, FOS, GOS, or other appropriate polysaccharide at 35% by weight, and buffer is present at 3% by weight. In an embodiment, lactose is present at 20% by weight, microbes at 1.47% by weight, FOS, GOS, or other appropriate polysaccharide at 40% by weight, and buffer is present at 3% by weight. In an embodiment, lactose is present at 20% by weight, microbes at 1.47% by weight, FOS, GOS, or other appropriate polysaccharide at 50% by weight, and buffer is present at 3% by weight. In an embodiment, lactose is present at 20% by weight, microbes at 1.47% by weight, FOS, GOS, or other appropriate polysaccharide at 60% by weight, and buffer is present at 3% by weight. In an embodiment, lactose is present at 20% by weight, microbes at 1.47% by weight, FOS, GOS, or other appropriate polysaccharide at 70% by weight, and buffer is present at 3% by weight. In an embodiment, lactose is present at 5% by weight, microbes at 1.47% by weight, FOS, GOS, or other appropriate polysaccharide at 90% by weight, and buffer is present at 3% by weight. In an embodiment, lactose is present at 3% by weight, microbes at 1.47% by weight, FOS, GOS, or other appropriate polysaccharide at 92% by weight, and buffer is present at 3% by weight. In an embodiment, lactose is present at 2% by weight, microbes at 1.47% by weight, FOS, GOS, or other appropriate polysaccharide at 93% by weight, and buffer is present at 3% by weight. In an embodiment, lactose is present at 1% by weight, microbes at 1.47% by weight, FOS, GOS, or other appropriate polysaccharide at 94% by weight, and buffer is present at 3% by weight. In an embodiment, lactose is present at 0.5% by weight, microbes at 1.47% by weight, FOS, GOS, or other appropriate polysaccharide at 95% by weight, and buffer is present at 3% by weight. If the microbes, FOS, GOS, or other, buffer and lactose do not make up 100% of the composition by weight, the remaining ingredients can be any suitable ingredients intended for consumption by a subject, e.g., human, including, but not limited to, ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### I. Additional Ingredients

Additional ingredients include ingredients to improve handling, preservatives, antioxidants, flavorings and the like. For example, in an embodiment, a prebiotic composition in powdered form can include flavorings such that when mixed in a liquid (e.g., water), the powder can flavor the liquid with various flavors such as grape, strawberry, lime, lemon, chocolate, and the like. In an embodiment, the compositions include microcrystalline cellulose or silicone dioxide. Preservatives can include, for example, benzoic acid, alcohols, for example, ethyl alcohol, and hydroxybenzoates. Antioxidants can include, for example, butylated hydroxyanisole (BHA), butylated hydroxytoluene (BHT), tocopherols (e.g., Vitamin E), and ascorbic acid (Vitamin C).

### Timing and Dosage of Probiotic and Treatments Known to Combat Musculoskeletal Disorders

In an embodiment, probiotic microbes, such as Lactobacillus brevis, Lactobacillus plantarum, Leuconostoc mesenteroides, and/or Pichia kudriavzevii, are given prior to beginning treatment with a drug typically prescribed for treatment of a musculoskeletal disorder.

Thus, in an embodiment, some or all doses of a treatment or drug are accompanied by a dose of microbes, e.g., live cultured bacteria and/or yeast, e.g., Lactobacillus brevis, Lactobacillus plantarum, Leuconostoc mesenteroides, and/or Pichia kudriavzevii. In an embodiment, microbes, e.g., Lactobacillus brevis, Lactobacillus plantarum, Leuconostoc mesenteroides, and/or Pichia kudriavzevii, are given initially with another treatment or drug, but then use of the microbes is discontinued. For example, the initial one, two, three, four, five, six, seven, eight, nine, ten, or more than ten days of treatment with a treatment or drug further comprises doses of microbes, with the use of microbes discontinued after that time. In an embodiment, microbes, (e.g., bacteria in yogurt), or microbes by themselves, can be given for the first two days of treatment; then the administration of microbes is discontinued. In another embodiment, probiotic microbes, either alone or in combination with other substances or treatments are used after the treatment with a drug or treatment for musculoskeletal disorders is terminated. The microbes can be taken for any suitable period after the termination of treatment with the drug and can be taken daily or at regular or irregular intervals. Doses can be as described below. Any suitable amount of probiotic per serving can be used that allows an effective microbiota in the GI as demonstrated by, for example, decreased symptoms of a given musculoskeletal disorder.

Examples of anti-osteoporosis combination partners are but are not limited to, bisphosphonates (alendronate, risedronate, ibandronate, zolendronate), biologics (denosumab, romosozumab), selective estrogen receptor mediators (Raloxifene), or anabolic agents (teriparatide, abaloparatide). In an embodiment, probiotic microbes, such as Lactobacillus brevis, Lactobacillus plantarum, Leuconostoc mesenteroides, and/or Pichia kudriavzevii, are given in conjunction with treatment, such as, but not limited to, bisphosphonates (alendronate, risedronate, ibandronate, zolendronate), biologics (denosumab, romosozumab), selective estrogen receptor mediators (Raloxifene), or anabolic agents (teriparatide, abaloparatide).

Examples of treatments for osteomyelitis that may be used in combination with compositions disclosed herein, include, but are not limited to surgery and antibiotics. In some embodiments, antibiotics are given intravenously. In some embodiments, antibiotics are given orally. Typically, compositions disclosed herein are given after cessation of antibiotic therapy; however, in some cases, a suitable antibiotic or a suitable delivery route of antibiotic allows for concurrent use of compositions described herein and antibiotic therapy.

Examples of treatments for delayed or non-union fractures include bone stimulation and surgery, such as bone grafts or fixations.

### Dosage Forms

### A. General

Compositions described herein include any suitable form, including liquid or powder. Powdered compositions can be as pure powder, or can be in the form of capsules, tablets, or the like. Powder can be packaged in bulk (e.g., in a container containing sufficient prebiotic or other substances for a subject to follow for an entire course of treatment with increasing doses of prebiotic, or a portion of a course of treatment), or as individual packets (e.g., packets containing a single dose of prebiotic plus other components, or packets containing the dose of prebiotic and other components needed for a particular day of a prebiotic treatment regimen). If packaged in bulk, the powder can be in any suitable container, such as a packet, sachet, canister, ampoule, ramekin, or bottle. The container can also include one or more scoops or similar serving devices of a size or sizes appropriate to measure and serve one or more doses of prebiotic and, optionally, other ingredients included in the powder. Liquid compositions contain prebiotic and, optionally, other ingredients, in a suitable liquid, e.g., water or buffer. Liquid compositions can be provided in bulk (e.g., in a container containing sufficient prebiotic or other substances for one subject in need thereof to follow an entire course of treatment with increasing doses of prebiotic, or a portion of a course of treatment), or as individual containers, such as cans, bottles, soft packs, and the like (e.g., containers containing a single dose of prebiotic plus other components in suitable liquid, or containers containing the dose of prebiotic and other components needed for a particular day of a prebiotic treatment regimen). The container can also include one or more measuring cups or similar serving devices of a size or sizes appropriate to measure and serve one or more doses of prebiotic and, optionally, other ingredients included in the liquid.

In an embodiment, compositions described herein comprise one or more excipients. In an embodiment, the one or more excipients comprise one or more antiadherents, one or more binders, one or more coatings, one or more disintegrants, one or more fillers, one or more flavors, one or more colors, one or more lubricants, one or more glidants, one or more sorbents, one or more preservatives, one or more sweeteners, or a combination thereof. In an embodiment, the antiadherent is magnesium stearate. In an embodiment, the one or more binders are cellulose, microcrystalline cellulose, hydroxypropyl cellulose, xylitol, sorbitol, maltitol, gelatin, polyvinylpyrrolidone, polyethylene glycol, methyl cellulose, hydroxypropyl methylcellulose, or a combination thereof. In an embodiment, the one or more coatings are a hydroxypropyl methylcellulose film, shellac, corn protein zein, gelatin, methyl acrylate-methacrylic acid copolymers, cellulose acetate succinate, hydroxy propyl methyl cellulose phthalate, hydroxy propyl methyl cellulose acetate succinate, polyvinyl acetate phthalate, methyl methacrylate-methacrylic acid copolymers, sodium alginate, stearic acid, or a combination thereof. In an embodiment, the one or more disintegrants are crosslinked polyvinylpyrrolidone (crospovidone), crosslinked sodium carboxymethyl cellulose (croscarmellose sodium), sodium starch glycolate, or a combination thereof. In an embodiment, the one or more fillers are calcium carbonate, magnesium stearate, dibasic calcium phosphate, cellulose, vegetable oil, vegetable fat, or a combination thereof. In an embodiment, the one or more flavors are mint, cherry, anise, peach, apricot, licorice, raspberry, vanilla, or a combination thereof. In an embodiment, the one or more lubricants are talc, silica, vegetable stearin, magnesium stearate, stearic acid, or a combination thereof. In an embodiment, the one or more glidants are fumed silica, talc, magnesium carbonate, or a combination thereof. In an embodiment, the one or more sorbents are fatty acids, waxes, shellac, plastics, plant fibers, or a combination thereof. In an embodiment, the one or more preservatives are vitamin A, vitamin E, vitamin C, retinyl palmitate, selenium, cysteine, methionine, citric acid, sodium citrate, methyl paraben, propyl paraben, or a combination thereof. In an embodiment, the one or more sweeteners are stevia, aspartame, sucralose, neotame, acesulfame potassium, saccharin or a combination thereof.

### B. Oral Dosage Forms and Components

In one aspect provided herein are methods and compositions formulated for oral delivery to a subject in need thereof. In an embodiment a composition is formulated to deliver a composition comprising a prebiotic to a subject in need thereof. In another embodiment, a pharmaceutical composition is formulated to deliver a composition comprising a prebiotic to a subject in need thereof. In another embodiment a composition is formulated to deliver a composition comprising prebiotic and a probiotic to a subject in need thereof. 1. Forms

In an embodiment, a composition is administered in solid, semi-solid, micro-emulsion, gel, or liquid form. Examples of such dosage forms include tablet forms disclosed in U.S. Pat. Nos. 3,048,526, 3,108,046, 4,786,505, 4,919,939, and 4,950,484; gel forms disclosed in U.S. Pat. Nos. 4,904,479, 6,482,435, 6,572,871, and 5,013,726; capsule forms disclosed in U.S. Pat. Nos. 4,800,083, 4,532,126, 4,935,243, and 6,258,380; or liquid forms disclosed in U.S. Pat. Nos. 4,625,494, 4,478,822, and 5,610,184; each of which is incorporated herein by reference in its entirety.

Forms of the compositions that can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients including freeze-dried plant material serving both as prebiotic and as a filler. Compressed tablets can be prepared by compressing in a suitable machine the active ingredient in a free-flowing form such as a powder or granules, optionally mixed with binders (e.g., povidone, gelatin, hydroxypropylmethyl cellulose), inert diluents, preservative, antioxidant, disintegrant (e.g., sodium starch glycolate, cross-linked povidone, cross-linked sodium carboxymethyl cellulose) or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. Tablets can optionally be provided with an enteric coating, to provide release in parts of the gut (e.g., colon, lower intestine) other than the stomach. All formulations for oral administration can be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds (prebiotics or probiotics) can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments can be added to the tablets or Dragee coatings for identification or to characterize different combinations of active compound doses.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethylene glycol or an oil medium, for example peanut oil, liquid paraffin, or olive oil.

Oral liquid preparations can be in the form of, for example, aqueous or oily suspensions, solutions, emulsions syrups or elixirs, or can be presented as a dry product for reconstitution with water or other suitable vehicle before use. Such liquid preparations can contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, acacia; nonaqueous vehicles (which can include edible oils), for example almond oil, oily esters such as glycerine, propylene glycol, or ethyl alcohol; preservatives, for example methyl or propyl p-hydoxybenzoate or sorbic acid, and, if desired, conventional flavoring or coloring agents.

In an embodiment, a provided composition includes a softgel formulation. A softgel can contain a gelatin-based shell that surrounds a liquid fill. The shell can be made of gelatin, plasticiser (e.g., glycerin and/or sorbitol), modifier, water, color, antioxidant, or flavor. The shell can be made with starch or carrageenan. The outer layer can be enteric coated. In an embodiment, a softgel formulation can include a water or oil soluble fill solution, or suspension of a composition, for example, a prebiotic composition, covered by a layer of gelatin.

An enteric coating can control the location of where a prebiotic composition is absorbed in the digestive system. For example, an enteric coating can be designed such that a prebiotic composition does not dissolve in the stomach but rather travels to the small intestine, where it dissolves. An enteric coating can be stable at low pH (such as in the stomach) and can dissolve at higher pH (for example, in the small intestine). Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac (trade name EmCoat 120 N, Marcoat 125); cellulose acetate phthalate (trade name aquacoat CPD®, SepifilmTM LP, Klucel, Aquacoat® ECD, and Metolose®); polyvinylacetate phthalate (trade name Sureteric®); and methacrylic acid (trade name Eudragit®).

In an embodiment, an enteric coated prebiotic composition is administered to a subject. In another embodiment, an enteric coated probiotic composition is administered to a subject. In another embodiment, an enteric coated probiotic and prebiotic composition is administered to a subject. In an embodiment, probiotic bacteria can be administered to a subject using an enteric coating. The stomach has an acidic environment that can kill probiotics. An enteric coating can protect probiotics as they pass through the stomach and small intestine.

Enteric coatings can be used to (1) prevent the gastric juice from reacting with or destroying the active substance, (2) prevent dilution of the active substance before it reaches the intestine, (3) ensure that the active substance is not released until after the preparation has passed the stomach, and (4) prevent live bacteria contained in the preparation from being killed because of the low pH-value in the stomach.

Enteric coatings can also be used for avoiding irritation of or damage to the mucous membrane of the stomach caused by substances contained in the oral preparation, and for counteracting or preventing formation or release of substances having an unpleasant odor or taste in the stomach. Finally, such coatings can be used for preventing nausea or vomiting on intake of oral preparations.

In an embodiment a prebiotic composition is provided as a tablet, capsule, or caplet with an enteric coating. In an embodiment the enteric coating is designed to hold the tablet, capsule, or caplet together when in the stomach. The enteric coating is designed to hold together in acid conditions of the stomach and break down in non-acid conditions and therefore release the drug in the intestines.

Softgel delivery systems can also incorporate phospholipids or polymers or natural gums to entrap a composition, for example, a prebiotic composition, in the gelatin layer with an outer coating to give desired delayed/control release effects, such as an enteric coating.

### Formulations of Softgel Fills can be at pH 2.5-7.5.

A softgel formulation can be sealed tightly in an automatic manner. A softgel formulation can easily be swallowed, allow for product identification using colors and several shapes, allow uniformity, precision and accuracy between dosages, be safe against adulteration, provide good availability and rapid absorption, and offer protection against contamination, light and oxidation. Furthermore, softgel formulations can avoid unpleasant flavors due to content encapsulation.

A composition comprising a softgel formulation can be in any of number of different sizes, including, for example, round, oblong, oval, tube, droplet, or suppositories.

In an embodiment a composition is provided in a dosage form which comprises an effective amount of prebiotic and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. In an embodiment the dosage form is a tablet, caplet, capsule or lollipop. In another embodiment, the dosage form is a liquid, oral suspension, oral solution, or oral syrup. In yet another embodiment, the dosage form is a gel capsule, soft gelatin capsule, or hard gelatin capsule.

In an embodiment, the dosage form is a gelatin capsule having a size indicated in Table 3.

TABLE 3
Gel Cap Sizes Allowable For Human Consumption:
Empty Gelatin Capsule Physical Specifications

| | Outer | Height or | Actual |
|---|---|---|---|
| | Diameter | Locked | Volume |
| | Size (mm) | Length (mm) | (ml) |
| 000 | 9.97 | 26.14 | 1.37 |
| 00 | 8.53 | 23.30 | 0.95 |
| 0 | 7.65 | 21.7 | 0.68 |
| 1 | 6.91 | 19.4 | 0.50 |
| 2 | 6.35 | 18.0 | 0.37 |
| 3 | 5.82 | 15.9 | 0.3 |
| 4 | 5.31 | 14.3 | 0.21 |
| 5 | 4.91 | 11.1 | 0.13 |

Note:
sizes and volumes are approximate.

In another embodiment a composition comprising a prebiotic is provided in effervescent dosage forms. The compositions can also comprise non-release controlling excipients.

In another embodiment, a composition comprising a prebiotic is provided in a dosage form that has at least one component that can facilitate release of the prebiotic. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment the prebiotic mixture is a plant or plant extract, either in solid or liquid form.

In another embodiment a composition comprising a prebiotic is provided in an enteric coated dosage form. The composition can also comprise non-release controlling excipients.

In another embodiment a composition comprising a prebiotic is provided in a dosage form for oral administration to a subject in need thereof, which comprises one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In an embodiment a composition comprising a prebiotic is provided in the form of enteric-coated granules, for oral administration. The compositions can further comprise cellulose, disodium hydrogen phosphate, hydroxypropyl cellulose, hypromellose, lactose, mannitol, and sodium lauryl sulfate.

In another embodiment a composition comprising a prebiotic is provided in the form of enteric-coated pellets, for oral administration. The compositions can further comprise glyceryl monostearate 40-50, hydroxypropyl cellulose, hypromellose, magnesium stearate, methacrylic acid copolymer type C, polysorbate 80, sugar spheres, talc, and triethyl citrate.

In an embodiment a composition comprising a prebiotic is provided in the form of enteric-coated granules, for oral administration. The compositions can further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In another embodiment a composition comprising a prebiotic can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The compositions provided herein can be in unit-dosage forms or multiple-dosage forms. Unit-dosage forms, as used herein, refer to physically discrete units suitable for administration to human or non-human animal subject in need thereof and packaged individually. Each unit-dose can contain a predetermined quantity of an active ingredient(s) sufficient to produce the desired therapeutic effect, in association with other pharmaceutical carriers or excipients. Examples of unit-dosage forms include, but are not limited to, ampoules, syringes, and individually packaged tablets and capsules. Unit-dosage forms can be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another embodiment the multiple dosage forms comprise different pharmaceutically active agents. For example a multiple dosage form can be provided which comprises a first dosage element comprising a composition comprising a prebiotic and a second dosage element comprising lactose or a probiotic, which can be in a modified release form.

In this example a pair of dosage elements can make a single unit dosage. In an embodiment a kit is provided comprising multiple unit dosages, wherein each unit comprises a first dosage element comprising a composition comprising a prebiotic and a second dosage element comprising probiotic, lactose or both, which can be in a modified release form. In another embodiment the kit further comprises a set of instructions.

In an embodiment, compositions can be formulated in various dosage forms for oral administration. The compositions can also be formulated as a modified release dosage form, including immediate-, delayed-, extended-, prolonged-, sustained-, pulsatile-, controlled-, extended, accelerated-, fast-, targeted-, programmed-release, and gastric retention dosage forms. These dosage forms can be prepared according to known methods and techniques (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126, which is herein incorporated by reference in its entirety).

In an embodiment, the compositions are in one or more dosage forms. For example, a composition can be administered in a solid or liquid form. Examples of solid dosage forms include but are not limited to discrete units in capsules or tablets, as a powder or granule, or present in a tablet conventionally formed by compression molding. Such compressed tablets can be prepared by compressing in a suitable machine the three or more agents and a pharmaceutically acceptable carrier. The molded tablets can be optionally coated or scored, having indicia inscribed thereon and can be so formulated as to cause immediate, substantially immediate, slow, controlled or extended release of a composition comprising a prebiotic. Furthermore, dosage forms of the invention can comprise acceptable carriers or salts known in the art, such as those described in the Handbook of Pharmaceutical Excipients, American Pharmaceutical Association (1986), incorporated by reference herein in its entirety.

In an embodiment, an effective amount of a composition comprising a prebiotic is mixed with a pharmaceutical excipient to form a solid preformulation composition comprising a homogeneous mixture of compounds described herein. When referring to these compositions as “homogeneous,” it is meant that the agents are dispersed evenly throughout the composition so that the composition can be subdivided into unit dosage forms such as tablets, caplets, or capsules. This solid preformulation composition can then be subdivided into unit dosage forms of the type described above comprising from, for example, 1 g to 20 mg of a prebiotic composition. A prebiotic composition can be formulated, in the case of caplets, capsules or tablets, to be swallowed whole, for example with water.

The compositions described herein can be in liquid form. The liquid formulations can comprise, for example, an agent in water-in-solution and/or suspension form; and a vehicle comprising polyethoxylated castor oil, alcohol, and/or a polyoxyethylated sorbitan mono-oleate with or without flavoring. Each dosage form comprises an effective amount of an active agent and can optionally comprise pharmaceutically inert agents, such as conventional excipients, vehicles, fillers, binders, disintegrants, pH adjusting substances, buffer, solvents, solubilizing agents, sweeteners, coloring agents, and any other inactive agents that can be included in pharmaceutical dosage forms for oral administration. Examples of such vehicles and additives can be found in Remington's Pharmaceutical Sciences, 17th edition (1985).

### 2. Manufacturing

The dosage forms described herein can be manufactured using processes that are well known to those of skill in the art. For example, for the manufacture of tablets, an effective amount of a prebiotic can be dispersed uniformly in one or more excipients, for example, using high shear granulation, low shear granulation, fluid bed granulation, or by blending for direct compression. Excipients include diluents, binders, disintegrants, dispersants, lubricants, glidants, stabilizers, surfactants and colorants. Diluents, also termed “fillers,” can be used to increase the bulk of a tablet so that a practical size is provided for compression. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders can impart cohesive qualities to a tablet formulation and can be used to help a tablet remain intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants can also facilitate tablet manufacture; non-limiting examples thereof include magnesium stearate, calcium stearate, stearic acid, glyceryl behenate, and polyethylene glycol. Disintegrants can facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Non-limiting examples of suitable glidants include silicon dioxide, talc, and the like. Stabilizers can inhibit or retard drug decomposition reactions, including oxidative reactions. Surfactants can also include and can be anionic, cationic, amphoteric or nonionic. If desired, the tablets can also comprise nontoxic auxiliary substances such as pH buffering agents, preservatives, e.g., antioxidants, wetting or emulsifying agents, solubilizing agents, coating agents, flavoring agents, and the like.

In an embodiment, a softgel formulation is made with a gelatin mass for the outer shell, and a composition including one or more substances, for example prebiotics and/or probiotics, for the capsule fill can be prepared. To make the gelatin mass, gelatin powder can be mixed with water and glycerin, heated, and stirred under vacuum. Additives, for example, flavors or colors, can be added to molten gelatin using a turbine mixer and transferred to mobile vessels. The gelatin mass can be kept in a steam-jacketed storage vessel at a constant temperature.

The encapsulation process can begin when the molten gel is pumped to a machine and two thin ribbons of gel are formed on either side of machine. These ribbons can then pass over a series of rollers and over a set of die that determine the size and shapes of capsules. A fill composition, for example a prebiotic and/or probiotic fill composition, can be fed to a positive displacement pump, which can dose the fill and inject it between two gelatin ribbons prior to sealing them together through the application of heat and pressure. To remove excess water, the capsules can pass through a conveyer into tumble dryers where a portion of the water can be removed. The capsules can then be placed on, for example, trays, which can be stacked and transferred into drying rooms. In the drying rooms, dry air can be forced over capsules to remove any excess moisture.

### 3. Release Formulations

Immediate-release formulations of an effective amount of a prebiotic composition can comprise one or more combinations of excipients that allow for a rapid release of a pharmaceutically active agent (such as from 1 minute to 1 hour after administration). In an embodiment an excipient can be microcrystalline cellulose, sodium carboxymethyl cellulose, sodium starch glycolate, corn starch, colloidal silica, Sodium Laurel Sulphate, Magnesium Stearate, Prosolve SMCC (HD90), croscarmellose Sodium, Crospovidone NF, Avicel PH200, and combinations of such excipients.

“Controlled-release” formulations (also referred to as sustained release (SR), extended-release (ER, XR, or XL), time-release or timed-release, controlled-release (CR), or continuous-release) refer to the release of a prebiotic composition from a dosage form at a particular desired point in time after the dosage form is administered to a subject. Controlled-release formulations can include one or more excipients, including but not limited to microcrystalline cellulose, sodium carboxymethyl cellulose, sodium starch glycolate, corn starch, colloidal silica, Sodium Laurel Sulphate, Magnesium Stearate, Prosolve SMCC (HD90), croscarmellose Sodium, Crospovidone NF, or Avicel PH200. Generally, controlled-release includes sustained but otherwise complete release. A sudden and total release in the large intestine at a desired and appointed time or a release in the intestines such as through the use of an enteric coating are both considered controlled-release. Controlled-release can occur at a predetermined time or in a predetermined place within the digestive tract. It is not meant to include a passive, uncontrolled process as in swallowing a normal tablet. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,556; 5,871,776; 5,902,632; and 5,837,284 each of which is incorporated herein by reference in its entirety.

In an embodiment a controlled release dosage form begins its release and continues that release over an extended period of time. Release can occur beginning almost immediately or can be sustained. Release can be constant, can increase or decrease over time, can be pulsed, can be continuous or intermittent, and the like. Generally, however, the release of at least one pharmaceutically active agent from a controlled-release dosage form will exceed the amount of time of release of the drug taken as a normal, passive release tablet. Thus, for example, while all of at least one pharmaceutically active agent of an uncoated aspirin tablet should be released within, for example, four hours, a controlled-release dosage form could release a smaller amount of aspirin over a period of six hours, 12 hours, or even longer. Controlled-release in accordance with the compositions and methods described herein generally means that the release occurs for a period of six hours or more, such as 12 hours or more.

In another embodiment a controlled release dosage refers to the release of an agent, from a composition or dosage form in which the agent is released according to a desired profile over an extended period of time. In an embodiment, controlled-release results in dissolution of an agent within 20-720 minutes after entering the stomach. In another embodiment, controlled-release occurs when there is dissolution of an agent within 20-720 minutes after being swallowed. In another embodiment, controlled-release occurs when there is dissolution of an agent within 20-720 minutes after entering the intestine. In another embodiment, controlled-release results in substantially complete dissolution after at least 1 hour following administration. In another embodiment, controlled-release results in substantially complete dissolution after at least 1 hour following oral administration. For example, controlled-release compositions allow delivery of an agent to a subject in need thereof over an extended period of time according to a predetermined profile. Such release rates can provide therapeutically effective levels of agent for an extended period of time and thereby provide a longer period of pharmacologic or diagnostic response as compared with conventional rapid release dosage forms. Such longer periods of response provide for many inherent benefits that are not achieved with immediate-release dosages. When used in connection with the dissolution profiles discussed herein, the term “controlled-release” refers to wherein all or less than all of the total amount of a dosage form, made according to methods and compositions described herein, delivers an active agent over a period of time greater than 1 hour.

In one aspect, controlled-release refers to delayed release of an agent, from a composition or dosage form in which the agent is released according to a desired profile in which the release occurs after a period of time.

When present in a controlled-release oral dosage form, the compositions described herein can be administered at a substantially lower daily dosage level than immediate-release forms.

In an embodiment, the controlled-release layer is capable of releasing 30 to 40% of the one or more active agents (e.g., prebiotic and/or probiotic) contained therein in the stomach of a subject in need thereof in 5 to 10 minutes following oral administration. In another embodiment, the controlled-release layer is capable of releasing 90% of the one or more active agents (e.g., prebiotic and/or probiotic) is released in 40 minutes after oral administration.

In some embodiments, the controlled-release layer comprises one or more excipients, including but not limited to silicified microcrystalline cellulose (e.g., HD90), croscarmellose sodium (AC-Di-Sol), hydroxyl methyl propyl cellulose, magnesium stearate, or stearic acid. In an embodiment, a controlled release formulation weighs between 100 mg to 3 g.

Pharmaceutical carriers or vehicles suitable for administration of the compounds provided herein include all such carriers known to those skilled in the art to be suitable for the particular mode of administration. In addition, the compositions can one or more components that do not impair the desired action, or with components that supplement the desired action, or have another action.

In another embodiment, an effective amount of the prebiotic is formulated in an immediate release form. In this embodiment the immediate-release form can be included in an amount that is effective to shorten the time to its maximum concentration in the blood. By way of example, certain immediate-release pharmaceutical preparations are taught in United States Patent Publication US 2005/0147710A1 entitled, “Powder Compaction and Enrobing,” which is incorporated herein in its entirety by reference.

The dosage forms described herein can also take the form of pharmaceutical particles manufactured by a variety of methods, including but not limited to high-pressure homogenization, wet or dry ball milling, or small particle precipitation (nano spray). Other methods to make a suitable powder formulation are the preparation of a solution of active ingredients and excipients, followed by precipitation, filtration, and pulverization, or followed by removal of the solvent by freeze-drying, followed by pulverization of the powder to the desired particle size.

In a further aspect the dosage form can be an effervescent dosage form. Effervescent means that the dosage form, when mixed with liquid, including water and saliva, evolves a gas. Some effervescent agents (or effervescent couple) evolve gas by means of a chemical reaction which takes place upon exposure of the effervescent disintegration agent to water or to saliva in the mouth. This reaction can be the result of the reaction of a soluble acid source and an alkali monocarbonate or carbonate source. The reaction of these two general compounds produces carbon dioxide gas upon contact with water or saliva. An effervescent couple (or the individual acid and base separately) can be coated with a solvent protective or enteric coating to prevent premature reaction. Such a couple can also be mixed with previously lyophilized particles (such as a prebiotic). The acid sources can be any which are safe for human consumption and can generally include food acids, acid and hydrite antacids such as, for example: citric, tartaric, amalic, fumeric, adipic, and succinics. Carbonate sources include dry solid carbonate and bicarbonate salt such as, preferably, sodium bicarbonate, sodium carbonate, potassium bicarbonate and potassium carbonate, magnesium carbonate and the like. Reactants which evolve oxygen or other gasses and which are safe for human consumption are also included. In an embodiment citric acid and sodium bicarbonate are used.

In another aspect the dosage form can be in a candy form (e.g., matrix), such as a lollipop or lozenge. In an embodiment an effective amount of a prebiotic is dispersed within a candy matrix. In an embodiment the candy matrix comprises one or more sugars (such as dextrose or sucrose). In another embodiment the candy matrix is a sugar-free matrix. The choice of a particular candy matrix is subject to wide variation. Conventional sweeteners such as sucrose can be utilized, or sugar alcohols suitable for use with diabetic patients, such as sorbitol or mannitol can be employed. Other sweeteners, such as the aspartame, can also be easily incorporated into a composition in accordance with compositions described herein. The candy base can be very soft and fast dissolving, or can be hard and slower dissolving. Various forms will have advantages in different situations.

A candy mass composition comprising an effective amount of the prebiotic can be orally administered to a subject in need thereof so that an effective amount of the prebiotic will be released into the subject's mouth as the candy mass dissolves and is swallowed. A subject in need thereof includes a human adult or child.

In an embodiment a candy mass is prepared that comprises one or more layers which can comprise different amounts or rates of dissolution of the prebiotic. In an embodiment a multilayer candy mass (such as a lollipop) comprises an outer layer with a concentration of the prebiotic differing from that of one or more inner layers. Such a drug delivery system has a variety of applications.

The choices of matrix and the concentration of the drug in the matrix can be important factors with respect to the rate of drug uptake. A matrix that dissolves quickly can deliver drug into the subject's mouth for absorption more quickly than a matrix that is slow to dissolve. Similarly, a candy matrix that contains the prebiotic in a high concentration can release more of the prebiotic in a given period of time than a candy having a low concentration. In an embodiment a candy matrix such as one disclosed in U.S. Pat. No. 4,671,953 or US Application Publication No. 2004/0213828 (which are herein incorporated by reference in their entirety) is used to deliver the prebiotic.

The dosage forms described herein can also take the form of pharmaceutical particles manufactured by a variety of methods, including but not limited to high-pressure homogenization, wet or dry ball milling, or small particle precipitation (e.g., nGimat's NanoSpray). Other methods useful to make a suitable powder formulation are the preparation of a solution of active ingredients and excipients, followed by precipitation, filtration, and pulverization, or followed by removal of the solvent by freeze-drying, followed by pulverization of the powder to the desired particle size. In an embodiment the pharmaceutical particles have a final size of 3-1000 μM, such as at most 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000 μM. In another embodiment the pharmaceutical particles have a final size of 10-500 μM. In another embodiment the pharmaceutical particles have a final size of 50-600 μM. In another embodiment the pharmaceutical particles have a final size of 100-800 μM.

In an embodiment an oral dosage form (such as a powder, tablet, or capsule) is provided comprising a prebiotic composition comprising 0.7 g of FOS, GOS, or other FOS, GOS, or other appropriate polysaccharide, 0.2 g of lactose, 0.01 g of glucose, 0.01 g of galactose, 0.1-0.2 g of a binder, 0.1-0.2 g of a dispersant, 0.1-0.2 g of a solubilizer, wherein the FOS, GOS, or other FOS, GOS, or other appropriate polysaccharide are composed of 1-25% disaccharides, 1-25% trisaccharides, 1-25% tetrasaccharides, and 1-25% pentasaccharides. The oral dosage form can be in the form of a powder, capsule, or tablet. Suitable amounts of binders, dispersants, and solubilizers are known in the art for preparation of oral tablets or capsules.

In another embodiment an oral dosage form (such as a powder, tablet or capsule) is provided comprising a prebiotic composition comprising 1-99.9% by weight of FOS, GOS, or other FOS, GOS, or other appropriate polysaccharide 0.5-20% by weight of lactose, 0.1-2% by weight of glucose, 0.1-2% by weight of galactose, 0.05-2% by weight of a binder, 0.05-2% by weight of a dispersant, 0.05-2% by weight of a solubilizer, wherein the FOS, GOS, or other FOS, GOS, or other appropriate polysaccharide are composed of 1-25% by weight disaccharides, 1-25% by weight trisaccharides, 1-25% by weight tetrasaccharides and 1-25% by weight pentasaccharides.

In another embodiment an oral dosage form (such as a powder, tablet, or capsule) is provided comprising a prebiotic composition comprising 1, 10, 20, 30, 40, 50, 60, 70, 80, 90, 95, 99.5 100% by weight of FOS, GOS, or other FOS, GOS, or other appropriate polysaccharide 0, 5, 10, 15 or 20% by weight of lactose, 0.1, 0.5, 1 or 2% by weight of glucose, 0.1, 0.5, 1 or 2% by weight of galactose, 0.05, 0.1, 0.5, 1 or 2% by weight of a binder, 0.05, 0.1, 0.5, 1 or 2% by weight of a dispersant, 0.05, 0.1, 0.5, 1 or 2% by weight of a solubilizer, wherein the FOS, GOS, or other FOS, GOS, or other appropriate polysaccharide are composed of 1, 5, 10, 15, 20 or 25% by weight disaccharides, 1, 5, 10, 15, 20, or 25% by weight trisaccharides, 1, 5, 10, 15, 20 or 25% by weight tetrasaccharides, and 1, 5, 10, 15, 20 or 25% by weight pentasaccharides.

In another embodiment, an oral dosage form is provided comprising a prebiotic composition, wherein the oral dosage form is a syrup. The syrup can comprise 1%, 5%, 10%, 15%, 20%, 25%, 30%, 35%, 40%, 45%, 50%, 55%, 60%, 65%, 70%, 75%, 80%, or 85% solid. The syrup can comprise 15%, 20%, 25%, 30%, 35%, 40%, 45% or 50% liquid, for example, water. The solid can comprise a prebiotic composition. The solid can be, for example, 1-96%, 10-96%, 20-96%, 30-96%, 40-96%, 50-96%, 60-96%, 70-96%, 80-96% or 90-96% prebiotic composition. The solid can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96% prebiotic composition. In an embodiment a prebiotic composition comprises FOS, GOS, or other FOS, GOS, or other appropriate polysaccharide. In another embodiment a prebiotic composition comprises FOS, GOS, or other FOS, GOS, or other appropriate polysaccharide and another prebiotic. In another embodiment a prebiotic composition comprises FOS, GOS or other and inulin or GOS and FOS.

In an embodiment, the softgel capsule is 0.25 mL, 0.5 mL, 1.0 mL, 1.25 mL, 1.5 mL, 1.75 mL, or 2.0 mL. In another embodiment, a softgel capsule comprises 0.1 g to 2.0 g of prebiotic composition. In another embodiment, a softgel capsule comprises 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 g of a prebiotic composition. In an embodiment the prebiotic composition comprises FOS, GOS, or other FOS, GOS, or other appropriate polysaccharide. In another embodiment the prebiotic composition consists essentially of FOS, GOS, or other FOS, GOS, or other appropriate polysaccharide. In another embodiment, a softgel capsule comprises FOS, GOS, or other FOS, GOS, or other appropriate polysaccharide and inulin or FOS.

In another embodiment, the prebiotic composition is delivered in a gelatin capsule containing an amount of FOS, GOS, or other FOS, GOS, or other appropriate polysaccharide within the ranges listed in Table 4. In another embodiment, the number of pills taken per day is within the ranges listed in Table 4.

TABLE 4
Exemplary GOS Dosing Units: Exemplary
GOS Composition Dosages in Gel Caps

| Size | GOS/Pill (g) | # pills per |
|---|---|---|
| 000 | 1-2 | 1-15 |
| 00 | 0.6-1.5 | 1-25 |
| 0 | 0.4-1.1 | 1-38 |
| 1 | 0.3-0.8 | 1-50 |
| 2 | 0.25-0.6 | 1-60 |
| 3 | 0.2-0.5 | 1-75 |
| 4 | 0.14-0.3 | 1-107 |

In another embodiment, a prebiotic composition is provided that does not contain a preservative. In another embodiment, a prebiotic composition is provided that does not contain an antioxidant. In another embodiment, a prebiotic composition is provided that does not contain a preservative or an antioxidant. In an embodiment a prebiotic composition comprising FOS, GOS, or other FOS, GOS, or other appropriate polysaccharide does not contain a preservative or an antioxidant.

In another embodiment, a prebiotic composition is formulated as a viscous fluid. In another embodiment, a prebiotic composition is formulated such that its water content is low enough that it does not support microbial growth. In an embodiment, this composition is an intermediate-moisture food, with a water activity between 0.6 and 0.85; in another embodiment this composition is a low-moisture food, with a water activity less than 0.6. Low-moisture foods limit microbial growth significantly and can be produced by one of ordinary skill in the art. For example, these products could be produced similarly to a liquid-centered cough drop. In another embodiment, a prebiotic composition is formulated as a viscous fluid without a preservative in a gel capsule. In another embodiment, a prebiotic composition comprising FOS, GOS, or other FOS, GOS, or other appropriate polysaccharide is a viscous fluid. In another embodiment, a prebiotic composition comprises a high percentage of FOS, GOS, or other FOS, GOS, or other appropriate polysaccharide that does not support microbial growth. In another embodiment, the prebiotic composition comprises FOS, GOS, or other FOS, GOS, or other appropriate polysaccharide and inulin or FOS.

In another embodiment, an oral dosage form is provided comprising a prebiotic composition, wherein the oral dosage form is a softgel. In an embodiment the softgel comprises a syrup. In an embodiment the syrup comprises a prebiotic composition. In an embodiment the prebiotic composition comprises FOS, GOS, or other FOS, GOS, or other appropriate polysaccharide. In another embodiment the prebiotic composition comprises more than 80% FOS, GOS, or other FOS, GOS, or other appropriate polysaccharide. In another embodiment the prebiotic composition comprises between 80-99.9% FOS, GOS, or other. In another embodiment the prebiotic composition comprises more than 80% FOS, GOS, or other FOS, GOS, or other appropriate polysaccharide. In another embodiment the prebiotic composition comprises 80, 81, 82, 83, 84, 85, 86, 87, 88, 89, 90, 91, 92, 93, 94, 95, 96, 97, 98, 99, or 99.9% FOS, GOS, or other FOS, GOS, or other appropriate polysaccharide.

In an embodiment a FOS, GOS, or other FOS, GOS, or other appropriate polysaccharide composition is formulated for delivery in a soft gel capsule. In an embodiment a FOS, GOS, or other FOS, GOS, or other appropriate polysaccharide composition formulated for delivery in a soft gel capsule is a high percentage FOS, GOS, or other FOS, GOS, or other appropriate polysaccharide composition, such as a 90-100% FOS, GOS, or other FOS, GOS, or other appropriate polysaccharide composition (e.g., 90, 91, 92, 93, 94, 95, 96, 97, 98, 99 or 100% FOS, GOS, or other FOS, GOS, or other appropriate polysaccharide composition by weight). In another embodiment a FOS, GOS, or other FOS, GOS, or other appropriate polysaccharide composition formulated for delivery in a soft gel capsule comprises 95% FOS, GOS, or other FOS, GOS, or other appropriate polysaccharide. In another embodiment a FOS, GOS, or other FOS, GOS, or other appropriate polysaccharide composition formulated for delivery in a soft gel capsule comprises 96% FOS, GOS, or other FOS, GOS, or other appropriate polysaccharide. In another embodiment, the FOS, GOS, or other FOS, GOS, or other appropriate polysaccharide composition is formulated such that its water content is low enough that it does not support microbial growth. In another embodiment, the FOS, GOS, or other FOS, GOS, or other appropriate polysaccharide composition is formulated as a viscous fluid without a preservative in a gel capsule. In another embodiment, the FOS, GOS, or other FOS, GOS, or other appropriate polysaccharide composition is formulated as a viscous fluid without an antioxidant in a gel capsule. In another embodiment the soft gel capsule comprises 0.1-2 g of a FOS, GOS, or other FOS, GOS, or other appropriate polysaccharide composition.

In another embodiment a prebiotic composition can be formulated as described, in U.S. Pat. No. 6,750,331, which is herein incorporated by reference in its entirety. A prebiotic composition can be formulated to comprise an oligosaccharide, a foaming component, a water-insoluble dietary fiber (e.g., cellulose or lignin), or a neutralizing component. In an embodiment a prebiotic composition can be in the form of a chewable tablet.

In an embodiment a foaming component can be at least one member selected from the group consisting of sodium hydrogencarbonate, sodium carbonate, and calcium carbonate. In an embodiment a neutralizing component can be at least one member selected from the group consisting of citric acid, L-tartaric acid, fumaric acid, L-ascorbic acid, DL-malic acid, acetic acid, lactic acid, and anhydrous citric acid. In an embodiment a water-insoluble dietary fiber can be at least one member selected from the group consisting of crystalline cellulose, wheat bran, oat bran, cone fiber, soy fiber, and beet fiber. The formulation can contain a sucrose fatty acid ester, powder sugar, fruit juice powder, and/or flavoring material.

Formulations of the provided invention can include additive components selected from various known additives. Such additives include, for example, saccharides (excluding oligosaccharides), sugar alcohols, sweeteners and like excipients, binders, disintegrators, lubricants, thickeners, surfactants, electrolytes, flavorings, coloring agents, pH modifiers, fluidity improvers, and the like. Specific examples of the additives include wheat starch, potato starch, corn starch, dextrin and like starches; sucrose, glucose, fructose, maltose, xylose, lactose and like saccharides (excluding oligosaccharides); sorbitol, mannitol, maltitol, xylitol and like sugar alcohols; calcium phosphate, calcium sulfate and like excipients; starch, saccharides, gelatin, gum arabic, dextrin, methyl cellulose, polyvinylpyrrolidone, polyvinyl alcohol, hydroxypropylcellulose, xanthan gum, pectin, gum tragacanth, casein, alginic acid and like binders and thickeners; leucine, isoleucine, L-valine, sugar esters, hardened oils, stearic acid, magnesium stearate, talc, macrogols and like lubricants; CMC, CMC-Na, CMC-Ca and like disintegrators; polysorbate, lecithin and like surfactants; aspartame, alitame and like dipeptides; silicon dioxide and like fluidity improvers; and stevia, saccharin, and like sweeteners. The amounts of these additives can be properly selected based on their relation to other components and properties of the preparation, production method, etc.

In an embodiment, a FOS, GOS, or other FOS, GOS, or other appropriate polysaccharide composition is a chewable oral dosage formulation. In an embodiment the chewable formulation can comprises between 1-99.9% FOS, GOS, or other FOS, GOS, or other appropriate polysaccharide. In an embodiment, a FOS, GOS, or other FOS, GOS, or other appropriate polysaccharide composition comprises 80% FOS, GOS, or other FOS, GOS, or other appropriate polysaccharide 5% L-ascorbic acid, 2% anhydrous citric acid, 3% sodium hydrogencarbonate, 3% calcium carbonate, 2% sucrose fatty acid, 3% fruit juice powder, and 2% potassium carbonate.

In another embodiment, a FOS, GOS, or other FOS, GOS, or other appropriate polysaccharide composition comprises 85% FOS, GOS, or other FOS, GOS, or other appropriate polysaccharide, 5% L-ascorbic acid, 3% sodium hydrogencarbonate, 2% sodium carbonate, 2% sucrose fatty acid ester, 2% fruit juice powder, and 1% potassium carbonate.

In another embodiment, a FOS, GOS, or other FOS, GOS, or other appropriate polysaccharide composition comprises 90% FOS, GOS, or other FOS, GOS, or other appropriate polysaccharide, 2% L-ascorbic acid, 1% anhydrous citric acid, 2% sodium hydrogencarbonate, 2% sodium carbonate, 2% sucrose fatty acid ester, and 1% potassium carbonate.

In another embodiment, a FOS, GOS, or other FOS, GOS, or other appropriate polysaccharide composition comprises 95% FOS, GOS, or other FOS, GOS, or other appropriate polysaccharide, 2% L-ascorbic acid, 1% sodium hydrogencarbonate, and 2% fruit juice powder. In another embodiment, a FOS, GOS, or other FOS, GOS, or other appropriate polysaccharide composition comprises 95% FOS, GOS, or other FOS, GOS, or other appropriate polysaccharide and 5% of L-ascorbic acid, anhydrous citric acid, sodium hydrogencarbonate, calcium carbonate, sucrose fatty acid, fruit juice powder, or potassium carbonate.

In another embodiment, a FOS, GOS, or other FOS, GOS, or other appropriate polysaccharide composition comprises 95% FOS, GOS, or other FOS, GOS, or other appropriate polysaccharide and 5% of L-ascorbic acid, anhydrous citric acid, sodium hydrogencarbonate, calcium carbonate, sucrose fatty acid, fruit juice powder, and potassium carbonate.

### Combination Therapy

In some embodiments, the compositions of the present invention can be used in conjunction with traditional treatments for a musculoskeletal disorder, such as an anti-osteoporosis or osteopenia therapy. In some embodiments, the present invention is administered together with at least one other therapy or agent. In some embodiments, the present invention is administered before the at least one other therapy or agent. In some embodiments, the present invention is administered after the at least one other therapy or agent. In other embodiments, the present invention is administered after cessation of another therapy or agent. The therapy includes, but is not limited to, approved therapies for osteoporosis, osteopenia, Paget's disease, stunting, osteoarthritis, osteomyelitis, delayed or on-union fractures, or any combination of the foregoing.

Some therapies for osteoporosis or osteopenia that are known in the art include: estrogen, estrogen agonists/antagonists (e.g. tamoxifen, raloxifene, toremifene, and bazedoxifine), bisphosphonates (e.g., alendronate, ibandronate, risedronate, and zoledronic acid), Denosumab, anabolic therapies (e.g. teriparatide, abaloparatide, romosozumab), and vitamin D. One of skill in the art would understand that the present invention may be used to supplement, increase efficacy of, or otherwise improve upon any of a number of known therapies for osteoporosis or osteopenia.

In certain embodiments, the methods disclosed herein comprise supplementation of Vitamin D by adjunct administration, e.g., concurrently or sequentially. In certain embodiments, the Vitamin D is administered daily, twice daily, thrice daily, every other day, or weekly. In certain embodiments, the Vitamin D is administered at a dose of 100-1,000 international units. In certain embodiments, the Vitamin D is administered at 400 international units. In certain embodiments, the Vitamin D is administered daily at 400 international units.

### Medical Foods

An alternate embodiment of the present invention is a formulation as a medical food. As used herein, a medical food is understood to be a form of dietary supplement.

The consuming public has come to understand that foods possess more than basic nutrition (protein, carbohydrate, fat, etc). For example, 95% of consumers agree that “certain foods have health benefits that go beyond basic nutrition and may reduce the risk of disease or other health concerns.” More than 50% of consumers believe that foods can replace the use of drugs. Replacing the use of drugs may have the benefit of reducing the incidence of adverse side effects suffered by patients following a pharmaceutical drug treatment regimen. In fact, medical foods are assumed to be generally safe, as people have historically consumed these foods safely in non-medical contexts.

The compositions of the disclosure may be administered under the supervision of a medical specialist, or may be self-administered. Medical foods could take the form of nutritional shakes or other liquids or meal replacements. Medical foods of the present invention could also take the form of a powder capable of being consumed upon addition to suitable food or liquid.

A medical food formulation of the present disclosure could confer benefits of a synthetic composition of microbes isolated from nutritionally beneficial plants, as well as the benefits of prebiotics, or other nutritionally beneficial inclusions, but not consumed to obtain nutrition from them but rather to provide a metabolic function different than a foodstuff. For example, medical foods of the disclosure may also include at least one vitamin, or vitamin precursor. Preferred vitamins possess antioxidant properties and include vitamins A, C and E, and/or their biochemical precursors. In an embodiment, the medical food of the disclosure includes at least one trace element, preferably selected from the group consisting of zinc, manganese and selenium. Medical foods of the disclosure also may include at least one additional antioxidant selected from the group consisting of carotenoids, N-acetylcysteine and L-glutamine. It is known to those of skill in the art how to construct medical foods containing these elements.

In certain aspects, medical foods disclosed herein include effective doses of microbes deemed useful for the indication(s) and effective doses of any vitamin, prebiotic, or other beneficial additive not consumed to obtain nutrition but to add a therapeutic benefit mediated by the production of SCFA or other immuno-stimulant molecules when passing through the GI tract.

Typically, the dietary supplements and medical foods of the present invention are consumed at least once daily, and preferably administered two times per day, optionally once in the morning and once in the afternoon or evening. A typical treatment regime for the dietary supplements or medical foods will continue for four to eight weeks. Depending on such factors as the medical condition being treated and the response of the patient, the treatment regime may be extended. A medical food of the present invention will typically be consumed in two servings per day as either a meal replacement or as a snack between meals.

Anyone perceived to be at risk from a musculoskeletal disorder, including osteoporosis or osteopenia, or already suffering from any of the foregoing, can potentially benefit from ingesting the compositions disclosed herein. According to the disclosure, it is believed to be possible to effectively ameliorate symptoms and conditions associated with musculoskeletal disorders with natural compounds, which do not show any severe side effects. Furthermore, the present methods are expected to be well-tolerated, for example without causing any discomfort or nausea, and simple to apply.

### Methods of the Invention

The administration of the microbial compositions disclosed herein (e.g., DMA compositions as described herein, e.g., a DMA composition comprising a prebiotic) can be accomplished orally or rectally, although administration is not limited to these methods. In some embodiments, the microbial composition is administered orally. In some embodiments, the microbial composition is delivered rectally. In some embodiments, the administration of the microbial composition occurs at regular intervals. In some embodiments, the administration occurs daily. In some embodiments, the administration occurs once daily, twice daily, three times daily, etc. In some embodiments, the administration of the microbial composition occurs regularly for at least 1 week, at least 2 weeks, at least 3 weeks, at least 4 weeks, at least 6 weeks, at least 2 months, at least 3 months, at least 4 months, at least 5 months, at least 6 months, at least 8 months, at least 10 months, at least 12 months, at least 14 months, at least 16 months, at least 18 months, or at least 2 years. In some embodiments, the administration of the microbial composition occurs regularly for about 1 week, about 2 weeks, about 3 weeks, about 4 weeks, about 6 weeks, about 2 months, about 3 months, about 4 months, about 5 months, about 6 months, about 8 months, about 10 months, about 12 months, about 14 months, about 16 months, about 18 months, or about 2 years. In a particular embodiment, administration of the microbial composition occurs twice daily for at least about 6 months or at least about 12 months.

The microbial composition can be administered via typical pharmacological means, such as slurries, capsules, microcapsules, or solutions, although means of administration are not limited to these methods. In some embodiments, an enteric capsule or enteric microcapsule is used. In some embodiments, the pharmaceutical composition involving the microbial composition described herein will be fresh or frozen prior to application. In some embodiments, said pharmaceutical composition will be lyophilized or otherwise treated to increase stability or otherwise obtain a benefit from said treatment. In some embodiments, the composition can be administered as a medical food.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1: Manufacture of a Defined Microbial Assemblage

This example describes the manufacture of a DMA comprising four strains of microorganisms isolated from foods.

### DMA Identity

Four microbial strains (strains of Lactobacillus brevis, Lactobacillus plantarum, Leuconostoc mesenteroides, and Pichia kudriavzevii) were isolated as described in U.S. patent application Ser. No. 16/694,876, which is herein incorporated by reference in its entirety. The genomes of these four strains were sequenced as described in 16/694,876 and aligned by nucleotide BLAST to 16S rRNA gene reference sequences and fungal ITS reference sequences from the NCBI database. The 16S rRNA gene sequences of the isolated bacterial strains L. brevis, L. plantarum, and L. mesenteroides correspond to the nucleotide sequences of SEQ ID NOs: 94, 100, and 93, respectively. The fungal ITS sequence of the isolated P. kudriavzevii strain corresponds to SEQ ID NO: 102. As shown in Table 5, all four strains were shown to have at least 98% nucleotide identity with an NCBI reference sequence.

TABLE 5
Nucleotide identity values of the 16S rRNA gene and ITS
sequences of strains against NCBI reference sequences

| | 16S rRNA | | % nucleotide |
|---|---|---|---|
| | sequence | Best-match | identity |
| | (bacteria) or | by 16S | between isolate |
| | fungal ITS | rRNA gene | sequence and |
| | sequence (P. | or ITS | reference |
| Species Name | kudriavzevii) | region | sequence |
| Lactobacillus | SEQ ID NO: 100 | L. plantarum | 98.91 |
| plantarum | | JCM 1149, | |
| | | ATCC 14917 | |
| Lactobacillus | SEQ ID NO: 94 | L. brevis | 99.8 |
| brevis | | ATCC 14869, | |
| | | DSM 20054 | |
| Leuconostoc | SEQ ID NO: 93 | Leuc. | 98.85 |
| mesenteroides | | mesenteroides | |
| | | DRC1506, | |
| | | JCM 31787 | |
| Pichia | SEQ ID NO: 102 | ATCC 6258 | 99.78 |
| kudriavzevii | | | |

The four strains identified in Table 5 were used in the formulation of a medical food product, DMA-04. In this exemplary embodiment, DMA-04 was formulated as an orally delivered synbiotic capsule containing a mixture of the live microbial probiotic strains identified in Table 5, in combination with prebiotics (oligofructose and dried, ground blueberry powder) and inert formulation ingredients (magnesium stearate and silicon dioxide). An exemplary formulation of DMA-04 is summarized in Table 6.

TABLE 6
A Formulation of DMA-04

| | Dose in | Daily Dose | Ingredient |
|---|---|---|---|
| | Each | in Four | in Each |
| | Capsule | Capsules | Capsule |
| Ingredient | (CFUs) | (CFUs) | (mg) |
| Lactobacillus | 7.5 × 10⁹ | 3.0 × 10¹⁰ | 10-30 |
| brevis | | | |
| Lactobacillus | 7.5 × 10⁹ | 3.0 × 10¹⁰ | 10-30 |
| plantarum | | | |
| Leuconostoc | 7.5 × 10⁹ | 3.0 × 10¹⁰ | 10-30 |
| mesenteroides | | | |
| Pichia | 1.25 × 10⁹ | 5.0 × 10⁹ | 60-120 |
| kudriavzevii | | | |
| Oligofructose | Not applicable | Not applicable | 150-200 |
| Dried, ground | Not applicable | Not applicable | 150-200 |
| blueberry powder | | | |
| Magnesium | Not applicable | Not applicable | 5-10 |
| stearate | | | |
| Silicon dioxide | Not applicable | Not applicable | 5-10 |
| Total | 2.375 × 10¹⁰ | 9.5 × 10¹⁰ | 400-500 |

### Mamifacture of DMA-04

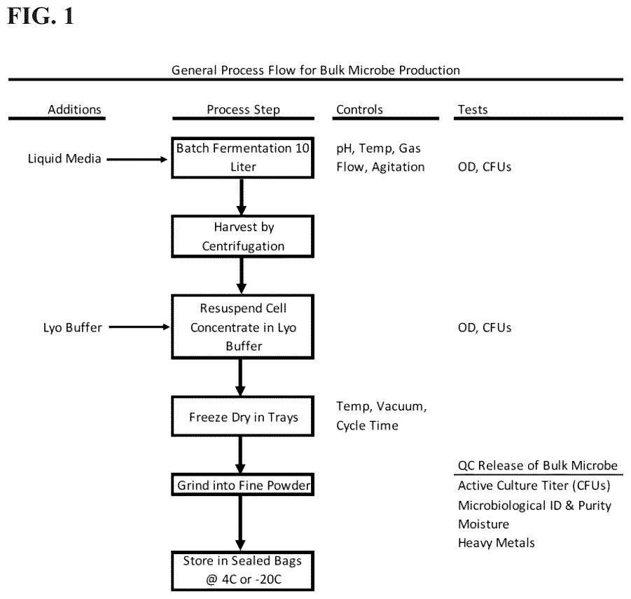

The manufacturing process for the four microbial strains included in DMA-04 involved bulk microbe production via fermentation. Bulk dry microbial biomass was produced according to the protocol summarized in FIG. 1. Briefly, microbial cultures were grown in liquid media via batch fermentation under controlled conditions of temperature, pH, gas flow, and agitation. The growth medium used for L. brevis, L. plantarum, and L. mesenteroides is summarized in Table 7, and the growth medium used for P. kudriavzevii is summarized in Table 8. The growth conditions used for each strain are summarized in Table 9. Feed parameters for P. kudriavzevii are summarized in Table 10. Cells were harvested by centrifugation, and supernatant was discarded. Concentrated cells were tested for cell titer and purity, and cells were subsequently resuspended in lyophilization buffer. The lyophilization buffer used for L. brevis, L. plantarum, and L. mesenteroides is summarized in Table 11, and the lyophilization buffer used for P. kudriavzevii is summarized in Table 12. Biomass was loaded into trays and frozen, and the frozen biomass was lyophilized under vacuum using primary and secondary drying steps. When necessary, lyophilized biomass was ground into a fine powder. The dry powdered biomass was loaded into bags, and the loaded bags were sealed and stored at 4° C. or −20° C.

TABLE 7
L. brevis, L. plantarum, and L. mesenteroides Growth Medium

| Medium Component | Concentration (g/L) |
|---|---|
| Peptone yeast | 13.0 |
| Yeast extract | 13.0 |
| D(+) Glucose | 20.0 |
| Di-Potassium hydrogen phosphate | 2.0 |
| Tween 80 | 1.0 |
| Sodium acetate | 0.8 |
| Magnesium sulfate | 0.1 |
| Manganese sulfate | 0.05 |
| Purified Water | Q.S. to 1 Liter |

TABLE 8
P. kudriavzevii Growth Medium

| Medium Component | Concentration (g/L) |
|---|---|
| Yeast extract | 30.0 |
| Dextrose | 30.0 |
| K₂HPO₄ | 2.0 |
| Sodium acetate | 5.0 |
| Ammonium Citrate | 2.0 |
| MgSO₄—7H₂O | 0.2 |
| MnSO₄—7H₂O | 0.03 |
| Antifoam 204 | 1 |

TABLE 9
Growth Conditions for DMA-04 Strains

| | L. | | L. | | L. | | P. | |
|---|---|---|---|---|---|---|---|---|
| Parameter | brevis | | plantarum | | mesenteroides | | kudriavzevii | |
| Culture | 30-37° | C. | 30-37° | C. | 30-37° | C. | 37° | C. |
| temper- | | | | | | | | |
| ature | | | | | | | | |
| Aeration/Gas | Anaerobic | | Anaerobic | | Anaerobic | | Aerobic | |
| Addition | (mix of CO₂, | | (mix of CO₂, | | (mix of CO₂, | | | |
| | hydrogen, | | hydrogen, | | hydrogen, | | | |
| | and nitrogen) | | and nitrogen) | | and nitrogen) | | | |
| Gas Flow | 0.1 | VVM | 0.1 | VVM | 0.1 | VVM | 0.5 | VVM |
| Rate | | | | | | | | |
| pH | 5.5-5.9 | | 5.5-5.9 | | 5.5-5.9 | | 5 | |
| Agitation | | | | | | | 200-1200 | rpm |
| Dissolved | | | | | | | 25% | |
| Oxygen | | | | | | | | |
| Inoculum | 0.1% | | 0.1% | | 0.1% | | 2% | |
| Harvest | 18 | hours | 12-18 | hours | 14-20 | hours | 60-72 | hours |
| Time | | | | | | | | |

TABLE 10
Feed Parameters for P. kudriavzevii

| | |
|---|---|
| Fermenter Fill Level | 70% of fermenter operating volume |
| Feed Volume | 30% of fermenter operating volume |
| Feed Medium | 50% glucose |
| Feed Start Time | 10 hours after inoculation |
| Initial Feed Rate | 0.25% fermenter operating volume per hour |
| Feed Type | Linear |
| Feed Rate Increases | Double flow rate at 34 hours and at 52 hours |

TABLE 11
L. brevis, L. plantarum, and
L. mesenteroides Lyophilization Buffer

| Buffer | Concentration |
|---|---|
| Component | (%) |
| Oligofructose | 6.0 |
| Trehalose | 1.0 |
| Ascorbic Acid | 0.1 |
| Cell Concentrate | 92.9 |

TABLE 12
P. kudriavzevii Lyophilization Buffer

| Buffer | Concentration |
|---|---|
| Component | (%) |
| Oligofructose | 5.0 |
| Sucrose | 5.0 |
| Ascorbic Acid | 0.1 |
| Cell Concentrate | 89.9 |

Powdered biomass of the individual microbial strains were manufactured and stored separately until being combined into the final product, DMA-04. Bulk biomass lots of individual strains were tested according to the methods and specifications summarized in Table 13. As shown in columns 4-7 of Table 13, all individual-strain biomass lots met the product quality specifications. The individual biomass lots were combined with prebiotics and flow aids to form DMA-04, e.g., according to the formulation specifications described in Table 6. The blended powder was packaged into size “0” capsules and filled into bottles (62 capsules per bottle). Bottles had a seal and screw cap closure. Labels were applied to bottles, and bottles were packaged into boxes for storage.

Combined DMA product lots were prepared at three dose levels: Low, Middle and High. These three product lots were tested for identity, potency and purity, as summarized in Table 14. As shown in columns 4-6 of Table 14, all product lots met the product quality specifications. Briefly, purity analyses included gross contamination tests, including visual observation of colony morphology on differential media and microscopic analyses of cell morphology. Samples were also tested for quantitation of certain heavy metals and microbial pathogens. Lastly, shotgun metagenomic DNA sequencing was used to detect genomic DNA from DMA-04 microbes and determine the presence of contaminating organisms present at levels of about 0.1-1%. As summarized in row 2 of Table 14, only the four DMA-04 microbial strains were present in the product lots, and the lots tested negative for contaminating microbes.

TABLE 13
Analytical Specifications for Each Strain in DMA-04

| | Target | | | L. | L. | |
|---|---|---|---|---|---|---|
| Attribute | Specification | Method | L. brevis | plantarum | mesenteroides | P. kudravzevii |
| Identity | Matches | 16S or ITS | Matches | Matches | Matches | Matches |
| | reference | sequencing | | | | |
| | strain | | | | | |
| Potency | Target | See Compendium | 340 | 600 | 500 | 19 |
| (Billion | titer +/− | of Methods for the | | | | |
| CFU) | 50% | Microbiological | | | | |
| | | Examination of | | | | |
| | | Foods, Chapter 20 | | | | |
| Water | <0.30 | Water Activity | 0.19 | 0.08 | 0.10 | 0.14 |
| Activity (for | | meter | | | | |
| dry biomass) | | | | | | |
| Total | <10,000/ | United States | <100/ | <100/ | <100/ | |
| Aerobic | gram | Pharmacopeia | gram | gram | gram | |
| Plate Count | | (USP) Test | | | | |
| | | <2021> | | | | |
| Total Non- | <5,000/ | International | | | | 100 CFU/ |
| Lactic Acid | gram | Organization for | | | | gram |
| Bacteria | | Standardization | | | | |
| Count | | (ISO) 13559 | | | | |
| E. coli | Negative/ | United States | Negative | Negative | Negative | Negative |
| | 10 gram | Pharmacopeia | | | | |
| | | (USP) Test | | | | |
| | | <2022> | | | | |
| Salmonella | Negative/ | USP Test <2022> | Negative | Negative | Negative | Negative |
| sp. | 10 gram | | | | | |
| S. aureus | Negative/ | USP Test <2022> | Negative | Negative | Negative | Negative |
| | 10 gram | | | | | |
| Yeast/mold* | <100/gram | USP Test <2021> | <10/ | <10/ | <10/ | N/A |
| | | | gram | gram | gram | |

*Not applicable to P. kudriavzevii, as the strain is a yeast.

TABLE 14
Batch Analysis of DMA-04

| | | | Result | Result | Result |
|---|---|---|---|---|---|
| | Target | | for Low | for Middle | for High |
| Parameter | Specification | Method | Dose Lot | Dose Lot | Dose Lot |
| Identity | Matches | 16S rRNA gene | Matches for | Matches for | Matches for |
| | reference | sequencing or ITS- | L. brevis, L. | L. brevis, L. | L. brevis, L. |
| | strains | 1 region | plantarum, L. | plantarum, L. | plantarum, L. |
| | | sequencing | mesenteroides | mesenteroides | mesenteroides |
| | | | and P. | and P. | and P. |
| | | | kudriavzevii | kudriavzevii | kudriavzevii |
| Total Lactic | Low: 4.6 × 10⁹ +/− | Solarea SOP0013 | 4.82 × 10⁹ | 2.33 × 10¹⁰ | 4.93 × 10¹⁰ |
| Acid Bacteria | 2.3 × 10⁹ | for Microbial | | | |
| Potency | Mid: 2.3 × 10¹⁰ +/− | Enumeration | | | |
| (CFU/ml) | 1.1 × 10¹⁰ | | | | |
| | High: 4.6 × 10¹⁰ +/− | | | | |
| | 2.3 × 10¹⁰ | | | | |
| P. kudriavzevii | Low: 2.58 × 10⁸ +/− | Solarea SOP0013 | 2.27 × 10⁸ | 1.44 × 10⁹ | 2.67 × 10⁹ |
| Potency | 1.29 × 10⁸ | for Microbial | | | |
| (CFU/ml) | Mid: 1.3 × 10⁹ +/− | Enumeration | | | |
| | 0.65 × 10⁹ | | | | |
| | High: 2.58 × 10⁹ +/− | | | | |
| | 1.29 × 10⁹ | | | | |
| E. coli | Negative/10 | USP Test <2022> | Negative | Negative | Negative |
| | gram | | | | |
| Salmonella sp. | Negative/10 | USP Test <2022> | Negative | Negative | Negative |
| | gram | | | | |
| S. aureus | Negative/10 | USP Test <2022> | Negative | Negative | Negative |
| | gram | | | | |
| Arsenic | <1 ppm | AOAC Official | <140 ppb | <140 ppb | <140 ppb |
| | | Methods | | | |
| | | 2011.19 & | | | |
| | | 993.14 | | | |
| Cadmium | <1 ppm | AOAC Official | <70 ppb | <70 ppb | <70 ppb |
| | | Methods | | | |
| | | 2011.19 & | | | |
| | | 993.14 | | | |
| Lead | <1 ppm | AOAC Official | <70 ppb | <70 ppb | <70 ppb |
| | | Methods | | | |
| | | 2011.19 & | | | |
| | | 993.14 | | | |
| Mercury | <1 ppm | AOAC Official | <70 ppb | <70 ppb | <70 ppb |
| | | Methods | | | |
| | | 2011.19 & | | | |
| | | 993.14 | | | |

### Example 2: Synergistic Acetate Production by Defined Microbial Assemblages

This example describes an in vitro analysis evaluating the ability of defined microbial assemblages to produce acetate as compared to monocultures of the individual strains.

The four constituent strains of the DMA DMA-04 were evaluated for their ability to produce short chain fatty acids (SCFAs) as monocultures versus their ability to produce SCFAs when combined into a single culture. Cryostocks of the four constituent DMA-04 strains described in Table 5 (L. brevis: SBS4254; P. kudriavzevii: SBS4263; L. mesenteroides: SBS4255; and L. plantarum: SBS4260) were prepared by individually growing each strain in DeMan-Rogosa-Sharpe broth (MRS) or in Potato dextrose broth (PDB). Cells were collected, washed with phospho-buffered saline (PBS), resuspended in fresh media containing either 10% DMSO or 18% glycerol, and frozen. The concentration of viable CFUs in each frozen cryostock was determined by dilution plating.

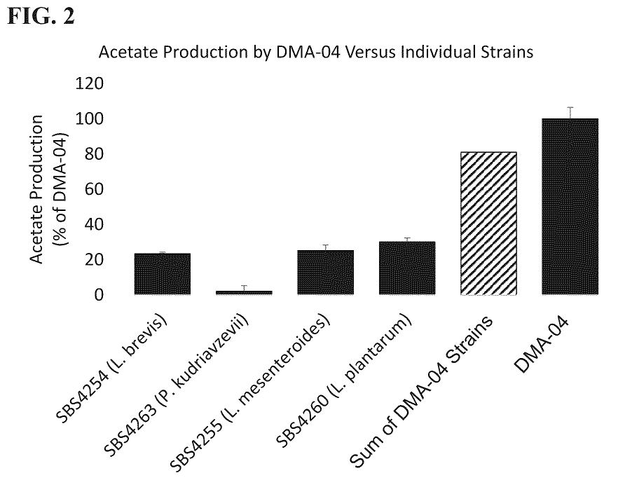

Each cryostock was defrosted, and cells were collected and washed with PBS. Washed cells were used to inoculate culture medium containing blueberry powder and oligofructose. Washed cells from each cryostock were used to inoculate separate cultures at a concentration of 2.5×10⁶ CFU/ml. For the “DMA-04” treatment, the culture medium was inoculated with 2.5×10⁶ CFU/ml of each of the four strains, for a total combined concentration of 1×10⁷ CFU/ml. The cultures were incubated anaerobically for 24 hours. Following the 24-hour incubation, cells from each culture were pelleted by centrifugation at 5000×g for 10 minutes, and culture supernatants were harvested for analysis. Final microbial titers were determined by dilution plating. Supernatant samples were acidified, and valeric acid was added as an internal control. Supernatant SCFAs were quantified by gas chromatography relative to a free fatty acid control (Restek). Acetate produced by the individual monocultures was quantified relative to acetate produced by the combined four-strain culture (FIG. 2). As shown in FIG. 2, the combination of the four individual DMA-04 strains into a single culture resulted in a synergistic improvement in acetate production.

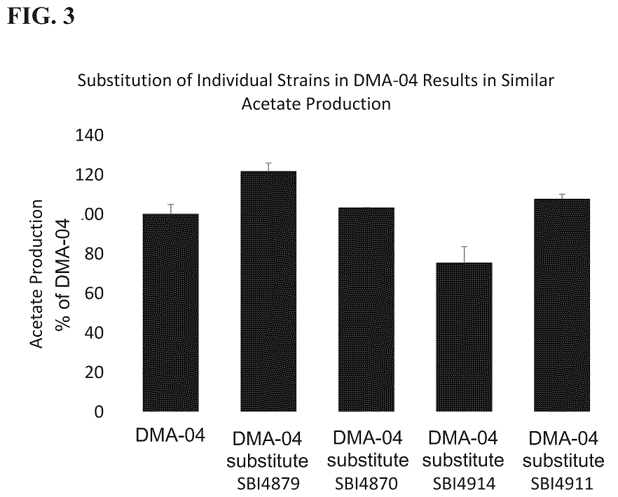

In order to confirm that the effects of DMA-04 are independent of the specific strains of species comprising DMA-04, four variants of the DMA-04 DMA were generated, wherein one of the four DMA-04 strains was substituted for a different microbial isolate of the same species: L. brevis SBS4254 was substituted for I . . . brevis SBI4879, P. kudriavzevii SBS4263 was substituted for P. kudravzevii SBI4870, L. mesenteroides SBS4255 was substituted for L. mesenteroides SBI4914, and L. plantarum SBS4260 was substituted for L. plantarum SBI4911. Each of the variants of DMA-04 were generated and tested for their ability to produce acetate according to the protocol described hereinabove. Acetate produced by each of the DMA-04 variant DMAs was quantified relative to acetate produced by DMA-04 (FIG. 3). As shown in FIG. 3, substituting any one of the DMA-04 microbial strains for a different strain of the same species resulted in a DMA which exhibited a similar level of acetate production as DMA-04, confirming that the effects of DMA-04 are independent of the specific strain.

### Example 3: Neurotransmitter Production by a Defined Microbial Assemblage

This example describes an in vitro analysis evaluating the ability of a defined microbial assemblage to produce neurotransmitters.

The ovarian hormones estrogen and progesterone influence neurotransmitters including gamma-aminobutyric acid (GABA), serotonin, and dopamine, impacting production and receptor expression. At perimenopause, the levels of these hormones decline as do their positive effects on neurotransmitters, resulting in degradations in cognition and mood. Microbes including bacteria and fungi are known to produce neurotransmitters such as GABA, melatonin, serotonin, and dopamine.

Microbially produced neurotransmitters including serotonin, melatonin, GABA, and dopamine are understood to affect host physiology and immunity through various mechanisms. Serotonin is synthesized from tryptophan (Trp) through a two-stage enzymatic reaction involving Trp hydroxylase and aromatic amino acid decarboxylase. In humans, approximately 90% of serotonin is produced by the enterochromaffin cells of the GI tract where it is understood to promote intestinal peristalsis In an anti-inflammatory role, serotonin has also previously been shown to induce T-cell differentiation into Tregs. Th17 cells are an inflammatory T-cell that secrete IL-17 and have been implicated in autoinflammatory diseases including but not limited rheumatoid arthritis. (Further, serotonin has previously been shown to reduce the production of IL-17 from Th17 cells and increase the production of IL-10 from Tregs, promoting an anti-inflammatory environment.) Melatonin is synthesized from serotonin in a two-step process. Melatonin's primary function is understood to be the regulation of circadian rhythm, but the compound has pleiotropic effects on the body. Potent anti-inflammatory effects of melatonin have previously been identified, including free radical scavenging and inhibition of inflammasome activation. Melatonin is also understood to play a role in the maintenance of bone, increasing bone regeneration and decreasing bone resorption. Several animal studies modeling osteoporosis have demonstrated the positive effects of melatonin supplementation on bone density. GABA is an inhibitory neurotransmitter in the central nervous system and exerts important functions in the immune system. GABA has previously been shown to decrease macrophage mediated inflammation and induce the production of Tregs. GABA has also previously been shown to decrease IL-1B mediated inflammation and increase production of tight junctions in epithelial cells, improving intestinal barrier function. Dopamine, a catecholamine, is abundantly present within the human intestinal tract in part due to microbial production. The bacterium Enterococcus faecalis has previously been shown to produce the neurotransmitter dopamine from the metabolite, L-3,4 dihydroxyphenylalanine (L-dopa). Furthermore, dopamine is recognized as a potent immunomodulatory compound. Dopamine is understood to reduce systemic inflammation through inhibition of the NLRP3 inflammasome, a proinflammatory signaling cascade, associated with robust secretion of proinflammatory mediators. Dopamine was also previously found to reduce neutrophil mediated reactive oxygen species production, and even inhibit neutrophil activation by the highly potent activator N-formyl-methionyl-leucyl-phenylalanine. Additionally, treatment with dopamine receptor agonists has previously been shown to reduce the levels of the pro-inflammatory cytokines IL-6 and IL-8 in serum.

Microbes and/or DMAs are evaluated for production of one or more neurotransmitters, including serotonin, melatonin, GABA, dopamine, or a combination thereof, using any appropriate method known in the art including, e.g., by quantifying amounts of one or more neurotransmitters in microbially-conditioned supernatant via ELISA. Alternatively, high performance liquid chromatography (HPLC) is used to quantify neurotransmitter production by potentially therapeutic microbes.

The four constituent strains of the DMA DMA-04 were evaluated for their ability to produce GABA and serotonin in vitro as monocultures versus their ability to produce SCFAs when combined into a single culture. Briefly, individual microbial strains were grown for 24-48 hours to achieve a high OD in brain heart infusion (BHI) or tryptic soy broth (TSB) for bacteria and potato dextrose broth (PDB) for yeast. Cultures were normalized by OD600 to achieve a uniform density and inoculated (10% final volume) into TSB containing 0.1% added tryptophan and grown for 48 hours. Culture supernatants were removed and analyzed by ELISA for GABA production (LS Bio) or serotonin production (ENZO) following the manufacturers' protocols. As summarized in Table 15, DMA-04 and its constituent strains were capable of producing GABA and serotonin.

TABLE 15
Production of Neurotransmitters
by DMA-04 and Constituent Strains

| | Genus and | GABA | Serotonin |
|---|---|---|---|
| Strain or DMA | Species | (pg/mL) | (pg/mL) |
| SBS4254 | Lactobacillus | 1.51 | 6.86 |
| | brevis | | |
| SBS4260 | Lactobacillus | 1.84 | 5.70 |
| | plantarum | | |
| SBS4255 | Leuconostoc | 1.58 | 5.59 |
| | mesenteroides | | |
| SBS4263 | Pichia | 1.56 | 3.60 |
| | kudriavzevii | | |
| DMA-04 | Not | 2.14 | 6.88 |
| | applicable | | |

### Example 4: A Defined Microbial Assemblage Protects Against Bone Loss in a Mouse Model of Osteoporosis

This example describes a mouse study to evaluate the ability of a Defined Microbial Assemblage to protect against an ovariectomy-induced decrease in Bone Mineral Density in a mouse model of osteoporosis.

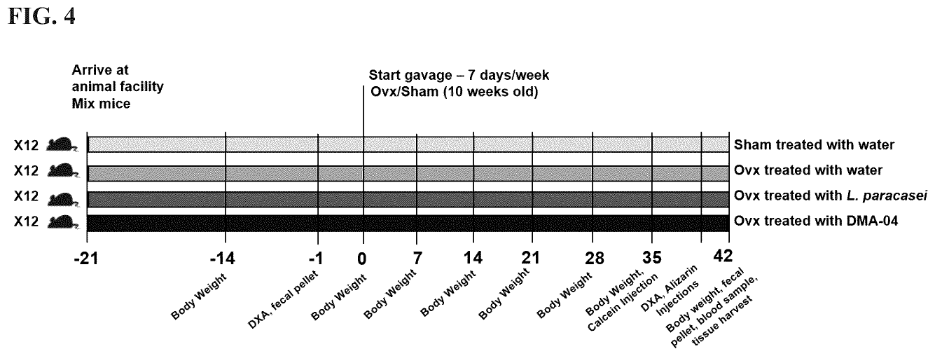

The ability of the DMA DMA-04 to protect against an ovariectomy-induced decrease in BMD in a mouse model of in a murine estrogen withdrawal model of osteoporosis was evaluated using the protocol summarized in FIG. 4. Briefly, 48 7-week-old female C57BL/6J mice (12 mice per treatment group; Jackson Laboratories) were transferred to a quarantine facility and acclimated to a new diet (Irradiated Teklad Diet 2016; Envigo) for 3 weeks. For the final 2 weeks of the 3-week acclimation period, the subjects' bedding was mixed in order to normalize subject microbiomes. One day or two days prior to the end of the acclimation period (“Day-1” or “Day-2”), fecal samples were collected and a DXA scan was performed to obtain a baseline BMD measurement. At the end of the 3-week acclimation period (“Day 0”), ovariectomy surgery was performed on the mice in three of the four treatment groups. Mice in the remaining treatment group were sham-treated as a control. Beginning on Day 0 and every day for 42 days thereafter, the ovariectomized mice were treated with either (1) DMA-04, (2) a single strain of Lactobacillus paracasei (representing an established probiotic), or (3) water as a negative control. The non-ovariectomized, sham-treated mice were likewise treated with water. Mice were injected with calcine on Day 33 (9 days prior to termination of the study), and mice were injected with alizarin on Day 40 (2 days prior to termination of the study). Body weights were recorded weekly beginning on Day 0. A second DXA scan was completed and a second fecal sample was collected 6 weeks after Day 0.

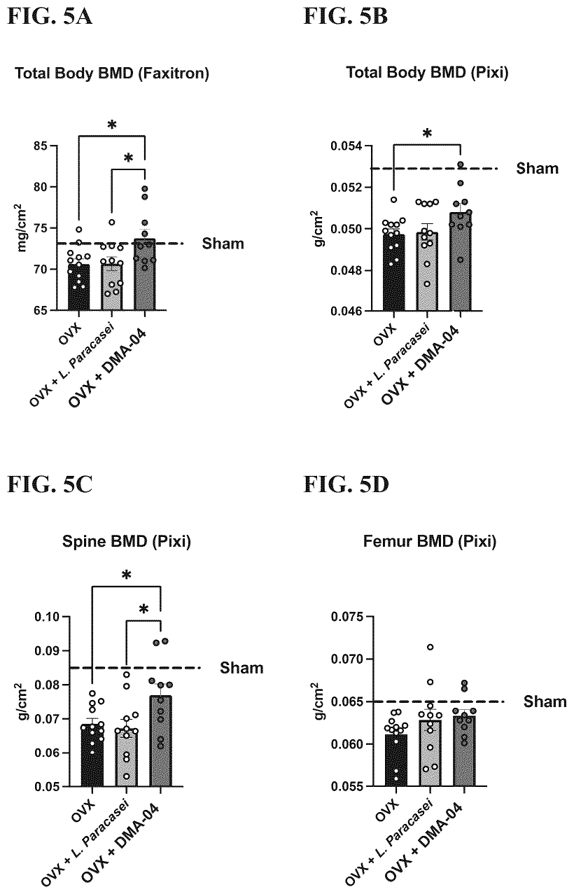

As shown in FIG. 5A-5B, treatment with the DMA DMA-04 was protective against an ovariectomy-induced loss in total body BMD, as measured by either a Faxitron DXA scan (FIG. 5A) or a Pixi DXA scan (FIG. 5B). By contrast, treatment with I . . . paracasei, a microbe which is commonly used as a probiotic, was not found to protect against a decrease in total body BMD. Treatment with DMA-04, but not L. paracasei, was likewise found to protect against an ovariectomy-induced decrease in spine BMD (FIG. 5C). These results confirm that DMA-04 protects from bone loss in subjects with reduced estrogen levels and indicate that DMA-04 can be used to prevent bone loss in menopausal subjects.

### Example 5: Food Trial to Evaluate Microbiome Changes Following Administration of a Defined Microbial Assemblage

This example describes a randomized, double-blind, placebo-controlled, parallel-arm food trial which demonstrates the successful engraftment of microbial strains into a human subject following administration of a DMA.

A group of 32 participants (adult men and women aged 18-70 years, with a Body Mass Index (BMI) between 18.5 and 35 kg/m2, with systolic blood pressure≤155 mm Hg, and with diastolic blood pressure≤95 mm Hg) was randomly divided into a placebo group and a treatment group. Sex was used as a stratification variable to ensure that sex was evenly distributed between groups. Trial participants, investigators, and all study staff involved in data collection were masked to participants' randomization assignments. Each participant in the treatment group was provided with 56 capsules containing the medical food synbiotic DMA DMA-04, which was manufactured, stored, and packaged as described in Example 1. The placebo group was provided with 56 capsules, each containing 500 mg of maltodextrin. All study products were identical in packaging and had the same appearance, taste, and texture. Participants consumed 2 capsules per day for 28 days, one with breakfast and one with dinner. Stool samples were collected for analysis immediately prior to beginning the study (Day 0), at the conclusion of the dosing period (Day 28), and an additional 28 days after the final dose (Day 56)

Gut microbiome composition was monitored by shotgun metagenomic analyses of stool samples collected on Day 0, Day 28, and Day 56. DNA was extracted from samples using the DNeasy 96 PowerSoil Pro QIAcube HT Kit (Qiagen 47021) according to manufacturer's instructions with a modification in the initial processing step on the QIAcube HT DNA extraction system (Qiagen 9001793). Mechanical lysis was performed with the PowerBead Pro beads (Qiagen 19311). Extracted DNA was quantitated using a high sensitivity dsDNA fluorometric assay (QuantIT, ThermoFisher, Q33120).

DNA libraries were generated using the Illumina DNA Prep (M) Tagmentation Kit (Illumina, 20018705) with IDT for Illumina DNA UD Index Sets A-D (Illumina 20027213-16) following manufacturer's instructions. Library quality was assessed by measuring DNA concentration using the high sensitivity dsDNA fluorometric assay (QuantIT, ThermoFisher, Q33120) and gel analysis. Individual libraries were pooled in equimolar amounts to create a sequencing pool. The pooled library was sequenced on the Illumina NovaSeq6000 using v1.5 300 bp PE sequencing reagents. DNA libraries were sequenced to a target 5 Gbp per sample with a minimum of 2 Gbp. Sequencing quality was assessed by Illumina sequencing metrics.

Quality control of raw reads was performed using the SolexaQA++package. Reads less than 50 bps and with a Phred quality score of less than 20 were discarded. Host sequencing reads were removed using Bowtie2 v 2.4.2. Nonpareil v3.0 was used to estimate the average coverage for each sequenced library. Taxonomic classification of metagenomic samples was performed using MetaPhlan3. Taxon relative abundances were calculated based on marker genes in a specific clade at different classification levels (species, genus, family). ITS coverage of P. kudravzevii in the metagenomes was calculated by mapping the metagenomics reads to the ITS sequence (coverage (X)=number of mapped reads*read length/ITS length). Functional profile was characterized using HUMAN3 with default parameters and reference pathways databases including UniRef, KEGG, UniPathway, and MetaCyc.

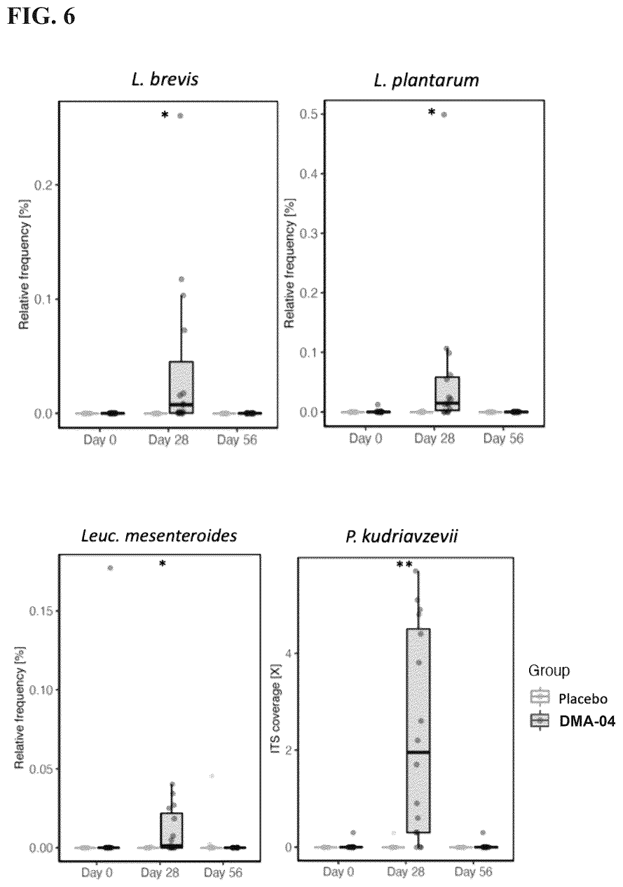

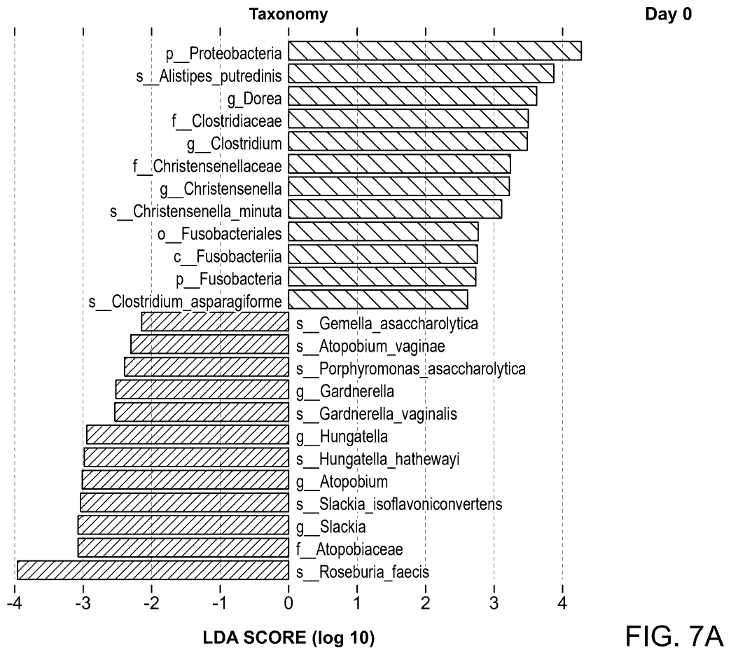

As shown in FIG. 6, DMA-04 strains were not detected in the treatment group participants or in the placebo group participants prior to administration of the test article (“Day 0”), with the exception of one participant with I . . . plantarum, one participant with I . . . mesenteroides, and one participant with P. kudriavzevii. All four DMA-04 strains increased variably in abundance after 28 days of administration of the DMA. Following a 28-day washout period (Day 56), all four strains decreased in abundance and were not detectable in any participants in the treatment group.

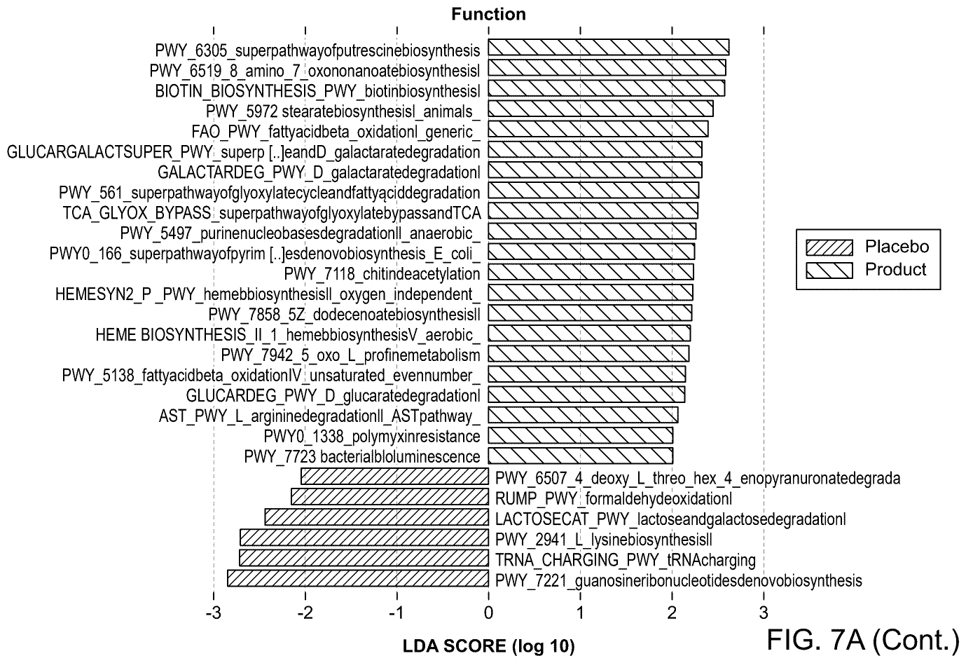

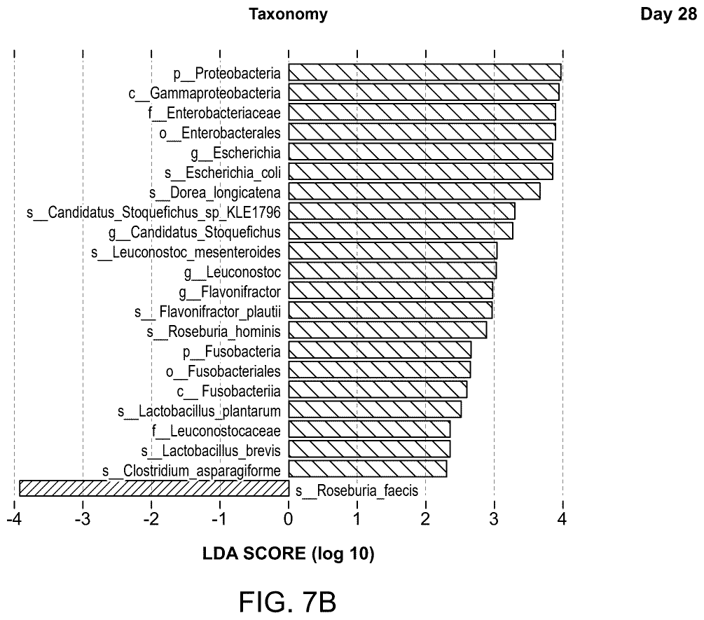

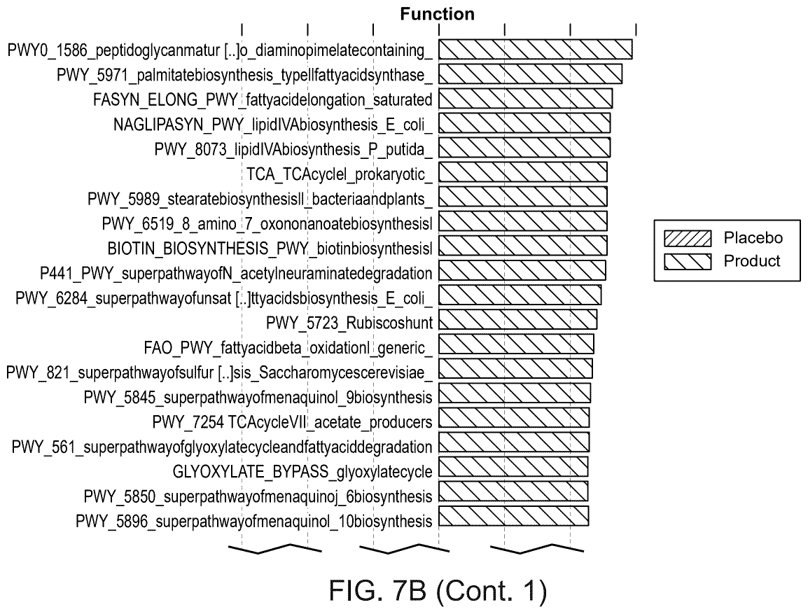

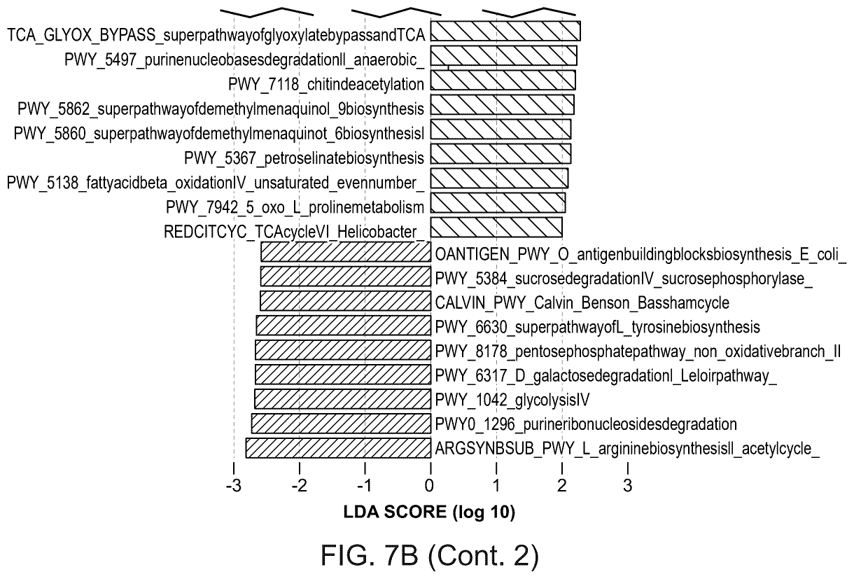

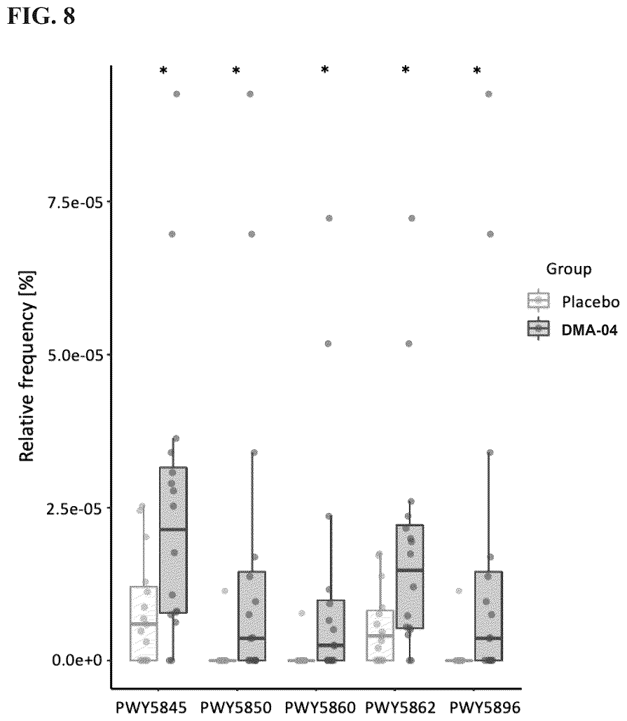

Linear Displacement Effect Size Analysis (LDA) was used to analyze changes in the abundance of microbial taxa and metabolic pathway genes between placebo and DMA-04 groups at different time points. On Day 0, 21 metabolic pathways were more abundant in the DMA-04 group than in the placebo group, and 6 metabolic pathways were more abundant in the placebo group than in the DMA-04 group (FIG. 7A). After 28 days of test product administration, 29 pathways were more abundant in the DMA-04 group, and 9 pathways were more abundant in the placebo group (FIG. 7B). In particular, pathways related to menaquinone (vitamin K2) production (super pathways of menaquinol-6,-9, and-10) were found to increase in abundance in the treatment group relative to the placebo group following administration of DMA-04 (FIG. 8). These data indicate that DMA-04 strains are able to pass through the intestinal tract and be identified in stool. These data also indicate that DMA-04 can provide relevant and potentially bone-protective functionalities, such as enhanced vitamin K2 production.

### Example 6: Clinical Study of a Defined Microbial Assemblage for the Management of the Metabolic Processes of Osteopenia and Symptoms of Menopause

This example describes a Phase 2/3 randomized, double blind, placebo-controlled food trial evaluating the efficacy of a Defined Microbial Assemblage (DMA) versus a placebo for the clinical dietary management of the metabolic processes of osteopenia and of symptoms of menopause.

### BACKGROUND

The human gut microbiome harbors a diverse set of microbial species that have coevolved with humans over millions of years. The composition of this ecosystem depends on both intrinsic and extrinsic factors, including host genetics and diet. The microorganisms inhabiting the gut benefit the host play a large role in shaping the immune system and play a large role in growth and development. However, alterations to the normal microbial flora have been implicated in various pathologies, including but not limited to allergic asthma, obesity, type 2 diabetes, osteoporosis, and Parkinson's disease. To manage these conditions, the use of health promoting probiotic microbes (live strains of bacteria and fungi that are orally administered and function along the gastrointestinal tract) and prebiotic dietary fibers (indigestible dietary fibers that specific commensal bacteria consume for fuel and metabolize into beneficial biproducts) have been described. Over the past decade, it has been increasingly appreciated that the gut microbiome has an important role in skeletal homeostasis.

The hormonal changes that occur during menopause, namely, reduction in estrogen, may affect bone density and vasomotor, and other physical, psychosocial, and sexual related symptoms. An approach that has shown promising results in the management of postmenopausal osteoporosis is the use of probiotics to maintain bone mass in postmenopausal women. Without wishing to be bound by theory, probiotics may increase the production of short chain fatty acid (SCFA) butyrate in the colon, leading to increased differentiation and proliferation of T-regulatory cells (Tregs). Tregs may affect the skeleton in several ways: (1) mitigating Th17-induced inflammation and osteoclastogenesis leading to decreased bone resorption and (2) Wnt10b-induced osteoblastogenesis leading to increased bone formation.

SCFAs present an intriguing therapeutic target, as they have been identified as anti-inflammatory mediators, decreasing inflammation via transcriptional regulation and immune cell activation. This is particularly relevant as postmenopausal osteoporosis has been identified as an inflammatory condition. In mice, ovariectomy (OVX) leads to decreased prevalence of beneficial commensal microbes in the gut microbiota, allowing other pathogenic bacteria to thrive. These alterations decrease intestinal barrier integrity, allowing intestinal microbes to penetrate the intestinal epithelium and lead to immune cell activation and endotoxemia, culminating in the systemic inflammation that leads to bone resorption. Estrogen deficiency promotes expansion and activation of effector T-cells including Th17 with an ensuing upregulation of osteoclastogenic factors such as TNF, IL-17, IFNγ, and RANKL, leading to a period of rapid bone resorption. In germ free mice, sex steroid deficiency does not stimulate T-cell expansion or TNF production in either the bone marrow or gut epithelium. Therefore, the microbiome is required for OVX-induced bone loss via expansion and activation of effector T-cell populations.

### Investigational Product and Placebo

DMA-04 is an orally delivered capsule containing a mixture of live microbial probiotic strains (a DMA containing Lactobacillus plantarum, Lactobacillus brevis, Leuconostoc mesenteroides, and Pichia kudriavzevii) with natural prebiotics and inert formulation ingredients. DMA-04 is manufactured and prepared according to the methods described in Example 1. The formulation of the investigational product DMA-04 as used in this Example is described in Table 16.

TABLE 16
An Exemplary Formulation of Investigational Product DMA-04

| | Dose in | Daily Dose | Ingredient |
|---|---|---|---|
| | Each | in Two | in Each |
| | Capsule | Capsules | Capsule |
| Ingredient | (CFUs) | (CFUs) | (mg) |
| Lactobacillus | 1.5 × 10¹⁰ | 3.0 × 10¹⁰ | 25-50 |
| brevis | | | |
| Lactobacillus | 1.5 × 10¹⁰ | 3.0 × 10¹⁰ | 25-50 |
| plantarum | | | |
| Leuconostoc | 1.5 × 10¹⁰ | 3.0 × 10¹⁰ | 25-50 |
| mesenteroides | | | |
| Pichia | 2.5 × 10⁹ | 5.0 × 10⁹ | 100-200 |
| kudriavzevii | | | |
| Oligofructose | Not applicable | Not applicable | 150-200 |
| Dried, ground | Not applicable | Not applicable | 150-200 |
| blueberry powder | | | |
| Magnesium | Not applicable | Not applicable | 5-10 |
| stearate | | | |
| Silicon dioxide | Not applicable | Not applicable | 5-10 |
| Total | 4.75 × 10¹⁰ | 9.5 × 10¹⁰ | 500-600 |

Briefly, bulk microbe probiotics are manufactured at multiple sites, and individual lots are blended together with prebiotic fibers, encapsulated, and packaged in high-density polyethylene (HDPE) bottles and caps with a desiccant sleeve and oxygen absorber. Product bottles are stored under refrigerated conditions prior to administration.

A placebo for use in the study consists of maltodextrin and food coloring. The placebo formulation has the same appearance as the investigational product. The placebo is packaged and prepared in the same manner as the investigational product: the placebo formulation is encapsulated and packaged in HDPE bottles and caps with a desiccant sleeve and oxygen absorber.

### Study Rationale

The hormonal changes that occur during menopause may affect bone density and vasomotor symptoms, as well as other physical, psychosocial, and sexual-related symptoms. In this study, the orally-dosed investigational product DMA-04 (prebiotic and probiotic) provided twice daily over a 12-month period helps support skeletal health and helps manage symptoms of menopause in otherwise healthy women in the early years of postmenopause (1-6 years post last menstruation).

### Objectives

A primary objective of this study is to evaluate the Change in Bone Mineral Density (BMD) measured by dual energy X-ray absorptiometry (DXA) at lumbar spine (2-4 evaluable levels L1-L4) between baseline and completion of the study period (12 months).

A secondary objective of this study is to evaluate change in BMD measured by DXA at femoral neck and hip between baseline and completion of the study period (12 months).

An additional secondary objective of this study is to evaluate the change in volumetric BMD (vBMD) measured by quantitative computed tomography (qCT) at lumbar spine (L1-L4) between baseline and completion of the study period.

An additional secondary objective of this study is to evaluate changes in biochemical markers of bone turnover (CTX, PINP) between baseline, 6 months, and completion of the study period.

An additional secondary objective of this study is to evaluate changes in levels of circulating inflammatory cytokines and markers of inflammation (CRP, IL-17, TNF, IL-1B, IL-4, RANKL, IFNγ) between baseline, 6 months, and completion of the study period.

An additional secondary objective of this study is to evaluate changes in the global Menopause Rating Scale (MRS) from baseline to months 2, 4, 6, 8, 10, and 12.

This study evaluates safety of the investigational product, as assessed by incidence of adverse events and serious adverse events. This study also evaluates tolerability of the investigational product, as assessed by Gastrointestinal Tolerability Questionnaire (GITQ) from baseline to month 12.

An additional secondary objective of this study is to evaluate changes in the global Menopause Rating Scale (MRS) from baseline to months 2, 4, 6, 8, 10, and 12.

### Study Design

### Overview of Study Design

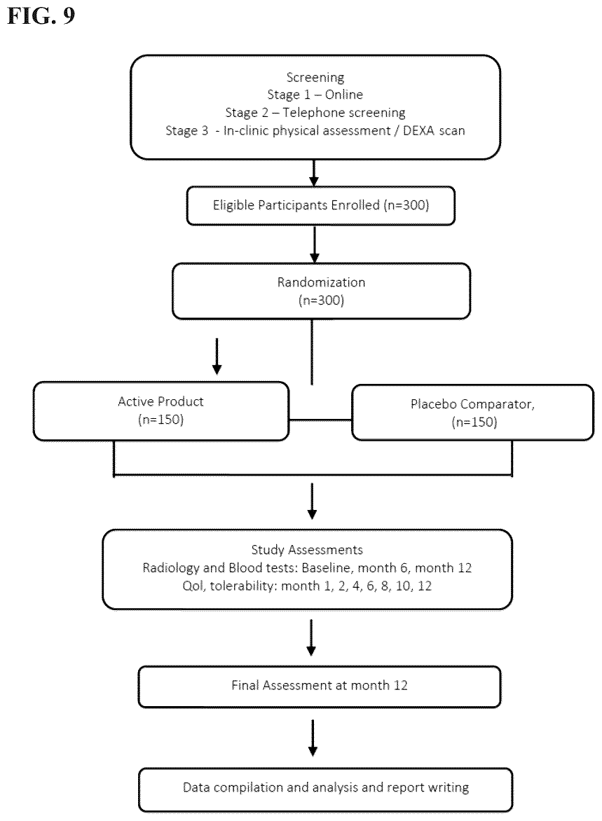

An interventional, prospective, randomized, double-blind, placebo-controlled, parallel study is conducted. The duration of the study is 12 months. The study design is summarized in FIG. 9.

Written informed consent is obtained from eligible study participants prior to study initiation. Approximately 300 otherwise healthy women in the early stages of postmenopause (1-6 years post last menstruation) are enrolled as participants. Baseline demographics, medical history, and vital signs are recorded. Eligible participants are randomly allocated into one of two study groups. One group receives the investigational product, and the other group receives the comparator product, the placebo. Blinded study products with a randomization code on the label are supplied to study sites, and clinical staff administering the study products dispense the study products to participants and record the randomization code assigned to each participant. The participants are assessed at regular intervals (as described below) to complete the efficacy and safety assessments. In the event that a participant enrolled for study participation decides to withdraw from the study at any point prior to the final interview (occurring at 12 months), all study procedures applicable at final interview are performed at the time of withdrawal, provided that the participant is willing to undergo the assessments. If the participant does not attend these assessments, they are included in the modified intent-to-treat population and not the Per protocol population.

### Historical and Clinical Assessments

Participant demographics, medical history, and concomitant medications are recorded. Vital signs such as weight, height, and blood pressure are also recorded. Adverse events (AE) and serious adverse event (SAE) are also recorded. Assessments include study assessments, such as radiology scans (DEXA and qCT scans), quality of life assessments, and diet/exercise assessments.

Assessments also include stool collections, which are carried out for gut microbiome analysis. Participants are provided with a stool collection kit, and follow instructions on the kit to collect and provide a stool sample for analysis.

Assessments also include pathology assessments. Blood tests are conducted and analyzed for both routine safety markers and for specified tests, including tests for inflammatory markers.

### Administration of Test Article

Study test articles (either DMA-04 or the placebo) are administered to all participants by an investigator and clinical staff as described in the schedule below. The study test articles are allocated to the participants randomly through the randomization process. The investigators provide the information regarding the administration of the study product to the participant. Participants are instructed to administer the study product two times daily (one capsule in the morning with breakfast and one capsule in the evening with dinner) regularly for the administration period of 12 months. If required, rescue medication is used for pain relief and the medicine and the dose is recorded.

The test article is supplemented by adjunct administration of Vitamin D at a daily dose of 400 international units.

### Validated Quality of Life Questionnaires

The gastrointestinal tolerability questionnaire (GITQ) and Menopause Rating Scale (MRS) are both used in this study.

The GITQ is a 12 item inventory of participant-reported gastrointestinal related discomfort. It is a patient self-report questionnaire assessment of the frequency and severity of gastrointestinal symptoms, e.g., gas and abdominal pain, calculated on a scale from Mild to Severe for all questions. This is used to determine whether use of the investigational product results in GI symptoms compared to the placebo.

The MRS score assesses menopausal symptoms. The scale was designed and standardized as a self-administered scale (a) to assess symptoms/complains of aging women under different conditions, (b) to evaluate the severity of symptoms over time, and (c) to measure changes pre- and post-menopause replacement therapy. The MRS has 11 items on a scale from no complaints (0) to very severe symptoms (4). Subscores are added to create a composite or total score. The hormonal changes that occur during menopause, namely reduction in estrogen, may affect vasomotor, and other physical, psychosocial and sexual related symptoms. The results of this study confirm that administration of the investigational drug DMA-04 as described herein reduces vasomotor symptoms associated with reduction in estrogen.

### Schedule of Study Procedures

The schedule of study procedures is outlined in Table 17, and the summary of study visits are described below.

TABLE 17
Schedule of Study Procedures

| | | | Week | Month 2, 4 | | Month 8, 10 | Month |
|---|---|---|---|---|---|---|---|
| | Screen | Baseline | 1, 2, 4 | (e-consult) | Month 6 | (e-consult) | 12 |
| Confirm Eligibility/ | X | X | | | | | |
| Enrollment | | | | | | | |
| Obtain Written | X | X | | | | | |
| Informed Consent | | | | | | | |
| Vital Signs (BP, height, | X | X | | | X | | X |
| weight) | | | | | | | |
| Blood Draw | X | X | | | X | | X |
| DXA Scan | X | | | | X | | X |
| qCT Scan | | X | | | | | X |
| Stool Sample | | X | | | X | | X |
| QOL Assessment | | X | | X | X | X | X |
| (MRS) | | | | | | | |
| Food freq and Exercise | | X | | | X | | X |
| Log | | | | | | | |
| Tolerability | | X | X | X | X | X | X |
| Assessment (GITQ) | | | | | | | |
| Safety Assessment | | | X | X | X | X | X |
| Start treatment | | X | | | | | X |
| Compliance | | | | X | X | X | X |

About 0 to 14 days prior to enrollment, potential participants are interviewed and screened for inclusion in the study. This includes: evaluation of inclusion/exclusion criteria, an explanation of study requirements, recordation of demographic and medical data (e.g. current illnesses and current medications/supplements), and sending the potential participants for pathology testing to analyze screening criteria.

Participants are interviewed prior to receiving the first dose (Week 0; “Baseline” in Table 17). This includes: confirming participant eligibility with respect to pathology and DEXA screening, confirming demographic and medical data (e.g., current illness and medications/supplements), obtaining informed consent, conducting a physical (including recording weight, height, and blood pressure), performing a radiology qCT scan, administering clinical questions, assessment by quality of life questionnaires, a blood draw for the remaining blood tests, randomization, study test product dispensing (including adjunct vitamin D), and provision of stool analysis kits (including a sample taken).

Participants are interviewed again at 1, 2, 4, and 8 weeks after the initial administration, and again every eight weeks thereafter. These interviews include: a compliance check, a safety and tolerability assessment by GITQ, and a menopause symptom assessment by MRS.

Participants are interviewed in a mid-study interview at about 24 to 26 weeks after the first administration. This interview includes: a compliance check, a safety and tolerability assessment by GITQ, a menopause symptom assessment check by MRS, a radiology DEXA scan, and a mid-study blood test.

Participants are interviewed again at study completion, about 48 to 52 weeks after the first administration. This interview includes: a compliance check; an assessment of changes to medication, illnesses, stresses, and life event changes; an assessment of weight and blood pressure; an adverse reactions check; assessment by GITO; assessment by MRS; a radiology DEXA scan; a qCT scan; a final-study blood test; and confirmation that pathology results are on file.

### Inclusion Exclusion Withdrawal Criteria

The study population consists of otherwise healthy women in early postmenopause who are able to provide written consent. Study participants meet all of the inclusion criteria and none of the exclusion criteria.

Inclusion criteria include: (1) providing written informed consent; (2) stated eligibility throughout the entire study period (12 months) and willingness to fulfill all details of the protocol, including having a DEXA scan, a CT scan, providing stool samples for analysis of the gut microbiome, and having blood tests; (3) in early postmenopause, at least 1 year but a maximum of 6 years since the last menstruation or since having a total hysterectomy; (4) at least six months since the last intake of hormone replacement therapy; (5) dual energy X-ray absorptiometry (DXA)-derived Bone Mineral Density (BMD) T-score of less than-2.49 at the lumbar spine (L1-L4), femoral neck, and total hip but no site with BMD≤-2.5; (6) body mass index between 18.5 and 32.5 kg/mg2; (7) normal levels of serum calcium (<11 mg/dL); and (8) normal cardiovascular parameters (systolic blood pressure≤155 mm Hg, diastolic blood pressure≤95 mm Hg) healthy and medication controlled.

Exclusion criteria include: (1) history of other bone disorders (e.g. Paget's disease or osteomalacia, osteogenesis imperfecta, osteoperosis, etc.); (2) history of cancer other than skin cancer, autoimmune disorders (rheumatoid arthritis, hashimoto's, graves' disease, etc.), type 2 diabetes, gastrointestinal disorders (ulcerative colitis, Crohn's disease, inflammatory bowel disease, irritable bowel syndrome), kidney disease or dysfunction or any other medical condition that could interfere with the conduct of the study; (3) history of chronic antibiotic use; (4) history of bariatric surgery; (5) history of partial colectomy; (6) women with spine abnormalities that would prohibit assessment of BMD; (7) Women who have undergone any joint replacement (hip, knee, etc.); (8) women who have undergone a partial hysterectomy; (9) women with untreated hyperparathyroidism; (10) Women previously treated with alcitonin, estrogens, estrogen derivatives, selective estrogen receptor modulators (SERMs), tibolone, progestins, anabolic steroids, or daily glucocorticoids in the past 6 months; (11) women treated with bisphosphonates or strontium in the past 5 years; (12) women previously treated with PTH, PTH analogs, gallium nitrate, romosozumab or denosumab; (13) per-oral use of corticosteroids; (14) smoking or use of nicotine products within the past 6-months; (15) any disease, that by the investigator's judgement, could interfere with the intestinal barrier function; (16) participation in other bone, diet, autoimmune, or gastrointestinal related clinical trials in the last 6 months; (17) desire and/or plans on changing current diet and/or exercise regime during the participation of this trial; (18) pregnancy or lactation; (19) consumption of dietary supplements (probiotics, prebiotics) in the month prior to the study or during the study, with the proviso that a participant willing to stop taking said supplements for 1 month may be enrolled following a 1-month washout period; and (20) consumption of antibiotics in the past two months, with the proviso that a participant placed on an antibiotic after enrollment in the study will be subject to a per protocol analysis.

A participant may be withdrawn from the study if any of the following conditions are met: (1) participant withdraws informed consent (with or without explanation, including those lost to follow-up); (2) adverse event or serious adverse event occurs (including a pathology abnormality) which, according to the investigator, makes study continuation impossible; (3) concomitant condition in which the prescribed additional therapy is prohibited by protocol as per the participant selection criteria; (4) protocol deviation, affecting the study results; (5) a participant becomes pregnant during the course of the study; (6) a participant may also be withdrawn based on the discretion of the investigator for any reason, if it is felt that her further continuation in the study will adversely affect him/her, or, in the interests of the study. The reason of discontinuation is reported. In the event than an enrolled participant decides to withdraw from the study at a point prior to the final interview, all study procedures applicable at final interview are completed, if possible.

### INCORPORATION BY REFERENCE

All references, issued patents, and patent applications cited within the body of the instant specification are hereby incorporated by reference in their entirety, for all purposes.

1.-36. (canceled)

37. A dietary supplement comprising a combination of two heterologous microbes, wherein the heterologous microbes in the dietary supplement consist of Lactobacillus brevis and Pichia kudriavzevii, wherein the dietary supplement is formulated for oral delivery.

38. The dietary supplement of claim 37, wherein the heterologous microbe Lactobacillus brevis comprises a 16S rRNA sequence having at least 97% identity to SEQ ID NO: 94.

39. The dietary supplement of claim 37, wherein the heterologous microbe Lactobacillus brevis comprises a 16S rRNA sequence having at least 98% identity to SEQ ID NO: 94.

40. The dietary supplement of claim 37, wherein the heterologous microbe Lactobacillus brevis comprises a 16S IRNA sequence having at least 99% identity to SEQ ID NO: 94.

41. The dietary supplement of claim 37, wherein the heterologous microbe Lactobacillus brevis comprises a 16S IRNA sequence SEQ ID NO: 94.

42. The dietary supplement of claim 37, wherein the heterologous microbe Pichia kudriavzevii comprises a fungal ITS sequence having at least 97% identity to SEQ ID NO: 102.

43. The dietary supplement of claim 37, wherein the heterologous microbe Pichia kudriavzevii comprises a fungal ITS sequence having at least 98% identity to SEQ ID NO: 102.

44. The dietary supplement of claim 37, wherein the heterologous microbe Pichia kudriavzevii comprises a fungal ITS sequence having at least 99% identity to SEQ ID NO: 102.

45. The dietary supplement of claim 37, wherein the heterologous microbe Pichia kudriavzevii comprises a fungal ITS sequence comprising SEQ ID NO: 102.

46. The dietary supplement of claim 37, further comprising a prebiotic selected from the group consisting of oligofructose, a dried fruit or vegetable powder, and combinations thereof.

47. The dietary supplement of claim 46, wherein the dried fruit or vegetable powder comprises dried blueberry powder.

48. The dietary supplement of claim 47, wherein the prebiotic comprises oligofructose and dried blueberry powder.

49. The dietary supplement of claim 37, wherein the dietary supplement is formulated as a medical food.

50. The dietary supplement of claim 37, wherein the heterologous microbes are co-formulated as a synthetic microbial consortium comprising about 1.0×10⁸ to about 1.0×10¹² CFU of each of the heterologous microbes.

51. The dietary supplement of claim 37, wherein the heterologous microbes are co-formulated as a synthetic microbial consortium comprising about 2.5×10⁹ to about 3.0×10¹⁰ CFU of each of the heterologous microbes.

52. The dietary supplement of claim 37, further comprising vitamin D.

53. The dietary supplement of claim 37, wherein the dietary supplement is formulated in a capsule.

54. A method of lessening a decrease in, maintaining, or improving bone health in a subject in need thereof, the method comprising administering to the subject an effective amount of the dietary supplement of claim 37.

55. The method of claim 54, wherein the subject has, is diagnosed with, or is at risk for one or more of the group consisting of: osteoporosis, osteopenia, osteoarthritis, suboptimal fracture healing, osteomyelitis, Paget's disease, stunting, and delayed or non-union fractures.

56. A method of producing the dietary supplement of claim 37, the method comprising co-formulating the heterologous microbes as a synthetic microbial consortia in a unit dose formulated for oral administration to a subject.
